(19) United States
(12) Patent Application Publication
(10) Pub. No.: US 2025/0346878 A1
(43) Pub. Date: Nov. 13, 2025

(54) EDITING OF DOUBLE-STRANDED DNA WITH RELAXED PAM REQUIREMENT

(71) Applicant: WUHAN UNIVERSITY, Hubei (CN)

(72) Inventors: Ying ZHANG, Wuhan (CN); Yajing SHI, Wuhan (CN)

(21) Appl. No.: 18/277,768

(22) PCT Filed: Feb. 21, 2022
(86) PCT No.: PCT/CN2022/077097

§ 371 Date: Aug. 17, 2023

(30) Foreign Application Priority Data
Feb. 19, 2021 (WO) PCT/CN2021/076930

(51) Int. Cl. C12N 9/22; C12N 9/12; C12N 9/78; C12N 15/11; C12N 15/90

(52) U.S. Cl. CPC C12N 9/226; C12N 9/1276; C12N 9/78; C12N 15/11; C12N 15/907; C12Y 207/07049; C07K 2319/00; C12N 2310/20

(57) ABSTRACT

Provided are new Cas proteins capable of gene editing with relaxed PAM requirement, or even does not require a PAM when targeting a negatively supercoiled double stranded DNA. It is further discovered that negatively supercoiled double stranded DNA in general reduces or even eliminates the PAM requirements for all Cas proteins. Accordingly, provided are compositions and methods for conducting gene editing, including base editing and prime editing, with relaxed or no PAM requirements.

### CROSS REFERENCE TO RELATED APPLICATIONS

This application is a U.S. National Stage Application under 35 U.S.C. 371 of International Application No. PCT/CN2022/077097, filed Feb. 21, 2022, which claims priority to Application No. PCT/CN2021/076930, filed Feb. 19, 2021, the content of each of which is incorporated by reference in its entirety into the present disclosure.

### SEQUENCE LISTING

The instant application contains a Sequence Listing which has been submitted in ASCII format via EFS-Web and is hereby incorporated by reference in its entirety. Said ASCII copy, created on Oct. 18, 2023, is named 347599_ST25.txt and is 56,737 bytes in size.

### FIELD OF THE DISCLOSURE

The present disclosure relates to the field of genetic engineering and more particularly to the area of gene modification.

### BACKGROUND

CRISPR/Cas (Clustered Regularly Interspaced Short Palindromic Repeats, and CRISPR-associated proteins) systems, present in 90% archaea and ˜40% of bacteria, serve as the adaptive immune machinery to protect the host from invading nucleic acids. The CRISPR/Cas systems contain CRISPR arrays, consisting of identical repeats that are interleaved with unique sequences as “spacer” acquired from foreign invaders, and their adjacent cas genes as their major components. When invaded by foreign DNA, CRISPR arrays are transcribed and processed into CRISPR RNA (crRNA). Cas proteins, guided by crRNA and a trans-activating RNA (tracrRNA), act to cleave target DNA that is complementary to the crRNA.

To distinguish self vs non-self, CRISPR systems have developed a preference of protospacer adjacent motif (PAM) located in the invader sequence. The PAM sequences are different among cas orthologues. PAM recognition and binding is essential in initiating local DNA unwinding and the subsequent cleavage. Current models for DNA unwinding include Cas9 searches and binds to a PAM site, followed by forming a directional R-loop extending from the PAM site. The PAM-interacting (PI) domain of Cas9 forms several hydrogen-bonds with the PAM bases and the deoxyribose-phosphate backbones, and this interaction is thought to serve as an anchoring point to initiate the target strand unwinding. Mutations in PAM can abolish R-loop formation and thus prohibit Cas9 activity.

Of characterized CRISPR/Cas systems, type II Cas9 protein, in particular, the Streptococcus pyogenes Cas9 (SpCas9), is the most robust and widely used in genome editing. SpCas9, guided by a programmable single-guide RNA (sgRNA) system, effectively cleaves the target DNA at sequences adjacent to an NGG PAM (N=A, T, C or G) and results in a blunt-ended double-stranded break. Based on SpCas9, new technologies such as Base editors and Prime editors enable the site-specific conversion of one or several DNA bases change into another, respectively. These new tools have brought great interests for developing new therapeutics as most genetic disorders are caused by point mutations and small deletion/insertion, and correcting these mutations is the only way to cure. Due to the inflexibility of targeted correction site, base editors and prime editor are limited to a restrictive editing window. Therefore, the PAM requirement has become a major barrier to identify highly efficient gRNAs. In order to increase the targeting range of SpCas9, several studies have taken protein engineering strategies to relax the PAM to NG or RY (R=A or G, Y=C or T), which collectively only cover ˜56% of sequences. Other Cas orthologs, such as Cas12a and SaCas9, are also used to a wider range of PAM sequences. While these variants have had a certain effect in expanding the potential targeting spacer of Cas9 protein, the targets carrying most non canonical PAMs is still a limitation of efficient genome editing.

### SUMMARY

The present disclosure reports the discovery of new Cas proteins capable of gene editing with relaxed protospacer adjacent motif (PAM) requirement, or even does not require a PAM when targeting a negatively supercoiled double-stranded DNA. It is further discovered that negatively supercoiled double stranded DNA in general reduces or even eliminates the PAM requirements for all Cas proteins. Accordingly, the disclosure provides compositions and methods for conducting gene editing, including base editing and prime editing, with relaxed or no PAM requirements.

In one embodiment, the present disclosure provides a method for editing a target nucleic acid, comprising contacting the target nucleic acid with a CRISPR-Cas system comprising: a Cas9 protein derived from Alicyclobacillus sp. or a functional variant thereof, wherein the functional variant has at least 70% sequence identity to the Cas9 protein derived from Alicyclobacillus sp., and a guide RNA comprising a guide sequence that hybridizes to a target sequence in the target nucleic acid, wherein the target sequence (a) is adjacent to a protospacer adjacent motif (PAM) comprising CNNN and RNNA, wherein R is A or G, and each N is independently A, T, C, or G, or (b) has an underwound topology.

In some embodiments, the Cas9 protein is derived from Alicyclobacillus tengchongensis, Alicyclobacillus hesperidum, or Alicyclobacillus Sacchari. In some embodiments, the Cas9 protein derived from Alicyclobacillus sp. comprises the amino acid sequence of SEQ ID NO: 84 or SEQ ID NO:85.

In some embodiments, the target sequence is negatively supercoiled DNA, bulged double stranded DNA, or Z-DNA. In some embodiments, the bulged DNA has one or more consecutive unpaired bases within positions 1-10 from 3′ of the complementary sequence of the target sequence. In some embodiments, the target sequence having underwound topology does not include the PAM.

Also provided, in one embodiment, is a mutant Cas9 protein, comprising (a) SEQ ID NO:84 with at least a mutation at a residue selected from the group consisting of E530, S531, L536, L602, D603, V604, T605, R1065, E1066, D1068, D1089, S1091, G1092, T1094, L1095, and T1096, or (b) a sequence having at least 70% sequence identity to SEQ ID NO:84 while retaining the mutation of (a).

In some embodiments, the mutation is selected from the group consisting of E530A, S531R, L536T, L602I, D603N, V604L, T605G, R1065A, E1066K, D1068K, D1068R, D1089A, D1089E, 51091A, G1092A, T1094A, L1095A, and T1096A.

In some embodiments, the mutation is at D1089 or T1096. In some embodiments, the mutation is D1089A or T1096A, or the combination thereof.

Also provided, in another embodiment, is a fusion protein comprising the mutant Cas9 protein and a nucleobase deaminase or a reverse transcriptase.

Further provided in one embodiment is a method for editing a target nucleic acid, comprising contacting the target nucleic acid with a CRISPR-Cas system comprising: a Cas protein with a corresponding protospacer adjacent motif (PAM) required for targeting a linear double stranded DNA, and a guide RNA comprising a guide sequence that hybridizes to a target sequence in the target nucleic acid, adjacent to a target PAM sequence, wherein the target sequence has an underwound topology and the target PAM sequence is not the corresponding PAM of the Cas protein.

In some embodiments, the Cas protein is SpCas9 and the corresponding PAM is NGG, wherein N is A, T, C or G. In some embodiments, the target PAM sequence is NAG or NGA.

In some embodiments, the Cas protein is FnCas9 and the corresponding PAM is NGG, wherein N is A, T, C or G. In some embodiments, the target PAM sequence is NGA.

In some embodiments, the Cas protein is SaCas9 and the corresponding PAM is NNGRRT, wherein each N is independently A, G, C or T, and each R is independently A or G.

In some embodiments, the target PAM sequence is NNGRRV, wherein V is A, C or G.

In some embodiments, the Cas protein is NmeCas9 and the corresponding PAM is NNNNGATT, wherein each N is independently A, G, C or T. In some embodiments, the target PAM sequence is NNNNGCTT, NNNNGTTT, NNNNGACT, NNNNGATA, NNNNGTCT, or NNNNGACA.

In some embodiments, the Cas protein is AsCas12a and the corresponding PAM is TTTV, wherein V is A, C or G. In some embodiments, the target PAM sequence is CTTV, TCTV, or TTCV

In some embodiments, the Cas protein is AtCas9 and the corresponding PAM is CNNN and RNNA, wherein each N is independently A, T, C or G, and R is A or G. In some embodiments, the target PAM sequence is any sequence other than CNNN and RNNA.

Another embodiment provides a method for editing a target nucleic acid, comprising contacting the target nucleic acid with a CRISPR-Cas system comprising: a Cas protein, and a guide RNA comprising a guide sequence that hybridizes to a target sequence in the target nucleic acid, wherein the Cas protein or the guide RNAis covalently or non-covalently coupled to an enzyme capable of changing the topology of the target nucleic acid.

In some embodiments, the Cas protein is fused to the enzyme. In some embodiments, the Cas protein and the enzyme each is fused to a corresponding protein partner which can bind to each other. In some embodiments, the two corresponding partners are a ligand and a corresponding receptor.

Also provided is a fusion protein comprising a Cas protein and an enzyme capable of changing the topology of a double stranded DNA.

In some embodiments, the enzyme is able to reduce positive supercoiling or increase negative supercoiling of the target nucleic acid. In some embodiments, the enzyme is selected from the group consisting of nonspecific E. coli heat-unstable (HU) protein, UvrD Helicase, Rep Helicase, PcrA Helicase, Dda Helicase, RecQ Helicase, eIF4A Helicase, WRN Helicase, NS3 Helicase, TRCF (Mfd) Helicase, Ltag Helicase, E1 Helicase, Rep Helicase, DnaB Helicase, gp41 Helicase, T7gp4 Helicase, Rho Helicase, DNA Helicase B (HELB), RecD Helicase, RecBCD Helicase, Pif1 Helicase, and Rrm3 Helicase.

In one embodiment of any of the methods, the guide RNA is a crRNA, a single guide RNA or a prime editing guide RNA (pegRNA). In some embodiments, the Cas protein is fused to a nucleobase deaminase or a reverse transcriptase. In some embodiments, the nucleobase deaminase is a dead nucleobase deaminase.

### BRIEF DESCRIPTION OF THE DRAWINGS

FIG. 1 shows in vitro characterization of AtCas9-catalyzed dsDNA cleavage. A. Schematic of CRISPR-Cas9 locus in A. tengchongensis species with Cas9, Cas1 and Cas2, tracrRNA and CRISPR array. Black diamond indicates spacer. B. Purified Cas9 orthologs, their respective mutants and reverse gyrase were analyzed on 8% SDS-PAGE and stained with coomassie brilliant blue. The predicted molecular weight of AtCas9 is ˜130 kD. C. In vitro cleavage assay to determine the optimal condition for AtCas9. Temperature range, kinetics, magnesium concentration and pH were assessed. 50 nM AtCas9 protein pre-complexed with 21a-crRNA and tracrRNA at 1:1 molar ratio, were incubated with 3 nM linear dsDNA substrate bearing 21a protospacer for 30 mins. The cleaved products are examined in 1% TAE gel. M, DNA marker; NC, negative control (only DNA template is added).

FIG. 2 shows in vivo PAM identification. A. Schematic representation of PAM identification in E. coli. The PAM library plasmid carrying a 21a protospacer and an 8-nt randomized PAM sequences was transformed into E. coli carrying plasmids harboring AtCas9 locus, SpCas9 (which is equivalent to SpyCas9 in the Figure) locus or a vehicle (which is equivalent to vector in the Figure) control. Plasmids are extracted and followed library preparation for deep sequencing. B. In vitro cleavage assay showing that increasing Mg²⁺ concentrations led to enhanced AtCas9 activity at 37° C. when incubated for 1 hour. C. Validate the effect of Mg²⁺ in regulating AtCas9's activity in E. coli. Transformed E. coli (described in FIG. 2A) were cultured in LB medium supplemented with indicated Mg²⁺ concentrations for 16 h at 37° C. Representative images showed that increasing Mg²⁺ led to cleared culture lysate (arrows), indicating that high level of Mg²⁺ promotes the cleavage of PAM library plasmid in E. coli. At and Sp represent E. coli expressing AtCas9 and SpCas9 locus respectively. D. Depletion of functional PAM plasmid libraries in E. coli by AtCas9 (top) and SpCas9 (bottom) proteins. Frequency of individual base at each position was normalized to vehicle control. AtCas9 exhibited no PAM preference whereas SpCas9 control exhibited a canonic NGG PAM.

FIG. 3 shows that AtCas9 displayed PAM-independent cleavage towards negative supercoiled DNA. A. Depletion of functional PAM library plasmids in E. coli by AtCas9 (left) and SpCas9 (right). Frequency of individual base at each position was normalized to control in which Cas9 was not expressed. B. E. coli cells harboring a AtCas9 expressing plasmid were transfected with targeted or mismatch protospacer PAM library plasmids (circles and squares) and cell densitities (OD600) were measured every an hour. For negative control, matched protospacer PAM library plasmids were transfected into control E. coli (positive triangle). For positive control, plasmids expressing AtCas9 locus and its matched protospacer with CNNA PAM were co-transfected into control E. coli (inverted triangle). At, AtCas9; mm ps, mismatch protospacer. Values and error bars reflect mean±s.d. n=3 independent experiments. C. Schematic representation of PAM library plasmids carrying the 21a protospacer and an 8-nt randomized PAM sequences. Three DNA topoisomers (linear, open circle and negative supercoil) were tested for in vitro cleavage. D. The cleaved products were resolved on 0.8% agarose gel. 100 nM Cas9 RNP and 3 nM dsDNA substrates were incubated at 1×buffer 16 for 30 min at 37° C. or 55° C. Noted that the three topoisomers migrated differently on the gel with supercoiled isomers being the fastest, then the linear isomers and the slowest open circle isomers. E. Quantification of cleavage efficiency showed that when complexed with cognate crRNA and tracrRNA, AtCas9 RNP had up to 100% cleavage efficiency towards negative supercoiled PAM library substrates at 55° C. and ˜60% efficiency towards linear or open circle counterparts. F. Sequence logo plot of PAM sequences for AtCas9 and AhCas9 using two spacers 21a, 21b and their respective PAM library substrates. Linear, open circle and negative supercoil represents three topological different library substrates. Neg.sc, negative supercoil. OC, open circle.

FIG. 4 shows that AtCas9 and AhCas9 displayed PAM-independent cleavage towards negative supercoiled DNA. A. In vitro cleavage of 21b-protospacer PAM library plasmids by AtCas9 RNP. B. Neighbor-joining phylogenetic tree based on 16S rRNA gene sequences showing the position of strain Alicyclobacillus tengchongensis with its closest relative Alicyclobacillus hesperidum in the genus Alicyclobacillus. C. In vitro cleavage of two different PAM library plasmids: 21a-protospacer and 21b-protospacer by AhCas9. When incubated with matched spacer, both AtCas9 (A, FIG. 3D) and AhCas9 (C) showed increased cleavage activity towards negative supercoiled substrate than linear counterparts. Neg.sc, negative supercoil; OC, open circle.

FIG. 5 shows negative supercoiled dsDNA exhibits broader PAM preference and enhanced cleavage than linear dsDNA. A. 50 nM AtCas9 RNP complex was incubated with 3 nM supercoiled or Nco I-linearized plasmid DNAbearing the complementary protospacer 21a with PAM (CATA for AtCas9) or mutated PAM (TATA for AtCas9). Controls include components minus one or a mismatch crRNA, showing the targeted cleavage requires the presence of matched crRNA and tracrRNA. B. In vitro cleavage of single-stranded DNA (ssDNA). 10 nM FAM-labeled ssDNA was incubated with 100 nM AtCas9 and its cognate crRNA in the presence or absence of tracrRNA. Cleaved products were resolved on 12% native PAGE. C. Kinetic analysis of cleavage efficiency using two different crRNAs 21a and 27m on their respective linear or supercoiled substrates with PAM (circles and positive triangle) or mutated PAM (squares and inverted triangle). Three independent experiments were performed and data were fitted to one phase exponential decay to calculate pseudo-first-order rate constant. D. The cleavage products of supercoiled substrates with PAM (CATA) or mutated PAM (TAGT) were gel purified, and subjected to restriction digestion with NcoI. The digested products were analyzed by 0.8% agarose gel. E. Sanger sequencing confirms site specific cleavage at three base-pair upstream of PAM in both strong and mutated PAM substrates. Asterisk indicates sequencing artifacts. Triangle indicates cleavage site. F. (left) 3 nM negative supercoiled and linearized dsDNA of 16 PAM combinations were incubated with 50 nM AtCas9 RNP. Each dot represents a different gRNA and data is presented as mean. (right) Evaluation of the cleavage efficiency of PAM and non-PAM substrates by AtCas9 in panel E left. The fold change obtained by dividing the cleavage efficiency of supercoil by the cleavage efficiency of linear is plotted. G. Effective cleavage of negative supercoiled plasmids with 16 PAM combinations in E. coli. (left) Schematic of natural transformation assays in E. coli. A mini AtCas9 CRISPR locus bearing 21a spacer was inserted into E. coli lacA locus via recombination. AtCas9 expressing E. coli were transformed with plasmids of sixteen PAM combinations bearing complementary sequence to 21a spacer or a mismatched sequence (EGFP). (right) Quantification of colony-forming units (CFU)/ml is presented for total cells (light grey bars) and kanamycin-resistant (black bars) transformants from six independent experiments. Student-t test is performed. ***p<0.0001.

FIG. 6 shows that AtCas9 and AhCas9 mediated PAM-independent cleavage requires both crRNA and tracrRNA. A. In vitro cleavage assay of linear or negative supercoiled DNA with wild-type or mutant PAM by AhCas9. The mutation site is underscored (i.e. first and last Ts in TAGT and first and last As in ATCA). B. Atcas9 or AhCas9 showed programmable PAM-independent cleavage towards supercoiled DNA. Five different spacers and their corresponding protospacer substrates with mutated PAM were used. Both AtCas9 and AhCas9 displayed up to 100% cleavage towards supercoiled DNA whereas reduced or no cleavage on linear substrates. S, negative supercoil; L, linear.

FIG. 7 shows kinetic analysis of DNA cleavage by AtCas9 and AhCas9. A-D, Comparison of rate constants for linearized plasmid DNA, supercoiled plasmid DNA either with WT PAM or MUT PAM. 21a spacer, 27m spacer (raw data for FIG. 5b) and its corresponding protospacer with either WT PAM or mutated PAM were used in AtCas9 mediated cleavage (A-B) whereas 21a spacer was used for AhCas9 (C). Mutated PAM was underscored (first “T” in “TATA” and first “A” in “ATAT” in FIG. 7A; “T” in “TGAC” and “A” in “ACTG” in FIG. 7B; two “Ts” in “TAGT” and two “As” in “ATCA” in FIG. 7C). PAMm indicates PAM mut. D. The data were fitted to one phase exponential decay to calculate pseudo-first-order rate constant. Three independent experiments were performed.

FIG. 8 shows that sequencing results show that AtCas9 produces blunt-end cleavage products. PCR generated linear substrate bearing protospacer EMX1-3 or 21a were incubated with AtCas9 RNP (left). Cleaved products were purified and sequenced (right). Sanger-sequencing traces showed that AtCas9 generated a blunt end at three base-pairs upstream of the PAM. The triangle indicates breakpoint of dsDNA. The 3′ terminal A or T overhang (asterisks) is an artifact of the sequencing reaction. TS: target strand, NTS: non-target strand

FIG. 9 shows preparation and cleavage of positive supercoiled plasmid. A. Positively supercoiled plasmid is generated by incubating negatively supercoiled DNA with reverse gyrase at various molar ratio of 1:1, 1:10, 1:50. Different topoisomers were analyzed in the agarose gel without chloroquine (left) or with 2 μM chloroquine (right). Reverse gyrase is a TYPE IV topoisomerase that introduces positive supercoils into DNA. Noted that negative supercoil displayed various topoisomers in the presence of chloroquine, a DNA intercalator known to regulate DNA topology by overwinding DNA. As the molar ratio of R.G./DNA increased, a homogeneous and compact positive supercoil isomer was observed at 50:1 ratio. Posi.sc, positive supercoil; Neg.sc., negative supercoil; R.G., reverse gyrase; σ, superhelical density. B. In vitro cleavage assay of three topological different substrates: Negative supercoil, linear and positive supercoil plasmid with different superhelical density (σ) described above. 21a protospacer is used.

FIG. 10 shows that DNA unwinding guided by its topological structure facilitates dsDNA cleavage. A. In vitro cleavage assay of three topological different substrates: negative supercoil, linear or positive supercoil plasmid with different superhelical density (a). Quantification of cleavage efficiency were performed (see FIG. 9B). N=3. Posi.sc, positive supercoil; Neg.sc., negative supercoil; R.G., reverse gyrase; σ, superhelical density. B. Schematic of DNA substrates used in C and D. A 2-3 nt length bulged DNA is generated by mutating the NTS strand at various position. TS, target strand; NTS, non-target strand; C. In vitro cleavage of bulged DNA substrates. Ctrl: control bulge; w/o, without. * indicated nicked products. D. Kinetics analysis of cleavage efficiency using bulged MUT PAM substrates (see FIG. 11). Quantification of cleaved products were done in triplicates. K_{cleave}±SD values for 0, 1-2, 3-4, 5-6, 19-20, 21-22, 23-25 bulge are 0.023±1.4 min⁻¹, 0.799±1.64 min⁻¹, 0.557±2.746 min⁻¹, 0.608±1.377 min⁻¹, 0.095±1.35 min⁻¹, 0.09±0.482 min⁻¹, 0.065±0.568 min⁻¹, respectively. E. (left) Schematic of three topoisomeric substrates: CC, hybridization of two complementary single-stranded (ss) DNA circles, is a Z-B chimeric circular DNA; CL, hybridization of complementary ssDNA circle and ssDNA, is a B-form open circle isomer; LL, hybridization of two complementary ssDNA, is a B-form linear isomer; Z-B, Z-B chimera; Z, Z-form DNA; B, B-form DNA. Z6 and Z7 represents two spacers that targeting Z-form DNA. (right) In vitro cleavage of Z-form DNA that carry WT or MUT PAM.

FIG. 11 shows kinetics analysis of cleavage efficiency using bulged mutated PAM substrates. A. 10 nM 120 bp oligonucleotides carrying 2-base bulge at non-targeting strand and mutant PAM (TATA) was incubated by 100 nM AtCas9 RNP at the indicate time, and cleavage product were resolved on 12% native PAGE (raw data for FIG. 10D, also see FIG. 10B for bulge illustration). As time increases, substrates containing the PAM proximal bulges were cleaved faster, whereas substrates with PAM distal bulges have a slower kinetics. B. Bulged mutated PAM dsDNA (120 bp) or target ssDNA (120 nt) were cleaved by AtCas9 and its cognate crRNA in the presence or absence of tracrRNA (see FIG. 10B for bulge illustration).

FIG. 12 verifies the B-Z DNA chimera by nuclease S1. (Left) 8% native PAGE showing the migration difference between three 89 nt-topoisomers. LL is prepared by hybridizing two complementary oligonucleotides and is a B-form linear isomer. CL is prepared by hybridizing a circular and a complementary linear oligonucleotide and is a B-form open circle isomer. CC represents hybridization of two complementary circular ssDNA and is a Z-B chimera circular isomer. (Right) Nuclease S1 recognized and digested B-Z junction in CC B-Z chimera. Digested products were resolved on 12% native PAGE. S1: S1 nuclease.

FIG. 13 shows that dAtCas9 showed higher binding affinity towards underwound DNA. A. Schematic diagram of AtCas9 domain structure showing RuvC and HNH nuclease domain mutation site. BH, arginine-rich bridge helix; REC, recognition lobe; L1, linker 1; L2, linker 2; WED, Wedge domain; TOPO, Topoisomerase-homology domain; CTD, C-terminal domain; HNH, HNH domain; D, Aspartic acid; H, Histidine; N, Asparagine; A, Alanine. B. (left) Electrophoretic mobility shift assay (EMSA) were carried out by incubating AtCas9 RNP with 25 nM Cy5-labeled oligonucleotides (50 bp) in buffer16 without Mg²⁺. SpCas9 showed sufficient binding whereas no binding is observed for AtCas9. (right) Catalytically dead AtCas9 (dAtCas9, D8A H617A N640A) were used for EMSA assay in the presence of Mg²⁺ C. Binding affinity of RNA-programmed dAtCas9 complex for 4 nM 5′ cy5-labeled oligonucleotides (50 bp) as measured by EMSA assay. The bulge substrate details are illustrated in FIG. 10B. KD±SD for PAM (0 bulge), 22.37±10.9 nM; KD±SD for mutated PAM (0 bulge), 248.2±4.5 nM; KD±SD for mutatedPAM (1-2 bulge), 29.46±12.2 nM. MUT PAM TATA and WT PAM CTAA was used. D. Higher binding affinity of AtCas9 RNPs towards negative supercoiled dsDNA than linear dsDNA. 1 nM cy5-labeled negative supercoil plasmid or NcoI-linearized plasmid containing PAM (CTAA) or mutant PAM (TGAC) motif were incubated with dAtCas9 RNP complex at indicated dosage. ProK, Proteinase K.

FIG. 14 shows torque residing within DNA topology is universal to the regulation of Cas activity. A. In vitro cleavage of linear or supercoiled PAM library substrates by divergent Cas systems. Each Cas ortholog in complex with its cognate dual-RNA showed ˜10-100 fold increase of cleavage activity towards negative supercoil than linear topoisomer. 50 nM RNP and 3 nM substrate were incubated at optimal temperature for 30 min. S, negative supercoil; L, linear; OC, open circle; w/o, without. B. Sequence logo plot of PAM sequences for SpCas9 using 21a spacer. Linear and negative supercoil represents two topological different library substrates. Neg.sc., negative supercoil. C. In vitro cleavage of linear and supercoiled WT PAM (CGG) or MUT PAM (CAG, CAT) substrates by SpCas9 RNP at 37° C. for 30 min. S, negative supercoil. L, linear. D. The bulge substrates described in FIG. 10B were cleaved by SpCas9.

FIG. 15 shows genome-editing activity of AtCas9 in mammalian cells. A. Cas9 uses HNH and RuvC domains to cleave the two DNA strands. Target strand (TS) and non-target strand (NTS) are labeled with cy3 and cy5, respectively. Cleavage products were analyzed on denature PAGE. B. (top) Schematic representation of protospacer 21a, tracrRNA, crRNA-21a complex. Minimal regions of crRNA and tracrRNA required for DNA cleavage are shown by shaded areas. Triangle, cleavage site; Black line, PAM. (bottom) Comparison of single-guide RNA (sgRNA) with dual RNA systems showed that engineered sgRNA has similar cleavage efficiencies using different spacers. C. Optimization of nuclear localization signals (NLSs) to enables nuclear import of AtCas9. Blue, Hoechst staining nuclei; Green, GFP reporter; Arrow, cytoplasmic localization; Arrowhead, nuclear localization. D. AtCas9 cleavage activity with different spacer lengths in two HEK293T reporter cell lines. Left reporter cells stably express p53-(+1 frame-shift)-EGFP and right reporter cells stably express EGFP. FACS analysis of GFP positive cells for p53 locus (left) or GFP negative cells for EGFP locus (right) were performed 5 days post transfection. Values and error bars reflect mean±s.d. n>=2 independent biological replicates. E. Schematic of AtCas9 sgRNA structure engineering. Grey shading indicates the engineered structure. Light grey indicates the spacer sequence. Gdl 200, 202, or 203 represents a vector expressing sgRNA scaffold varies in repeat-anti-repeat or stem loop1 or stem loop2 structures. F. AtCas9 cleavage activity against EGFP locus with three sgRNA variants. Values and error bars reflect mean±s.d. of n=5 independent biological replicates. G. Indel activity of endogenous sites in HEK293T cells bearing CNNAPAM with AtCas9. Editing efficiency were assessed by TIDE analysis. The sgRNAs targeting the VEGFA site and FANCF site are covered with different level of shades, respectively. Black shading indicates the assay detection limit of 4%. H. Schematic of mammalian expression constructs for AtCas9 cytosine base editor (CBE), which contain three main components: a cytidine deaminase (APOBEC-1), a Cas nickase (AtCas9 D8A) and uracil glycosylase inhibitor (UGI). I. C-to-T base editing efficiency of AtCas9 at four different loci (VEGFA, RUNXI, C-MYC and EGFP) harboring sixteen PAM combinations. Each dot represents the editing efficiency of one sgRNA. The data was presented as mean value, and the gray shade are violin plots. n>=2 independent biological replicates.

FIG. 16 shows engineering and characterization of AtCas9 PAM-Interacting (PI) domain variants. A. Different PAM substrates were cleaved using AtCas9 PI domain variants. The mutant amino acid residues and their positions used were indicated. Wild-type and mutant PAM are shown in green and red respectively. B. Sequence alignment of the PAM-Interacting domain from typeII-C Cas9 orthologous. Sequences of NmeCas9, AtCas9 and AhCas9 were aligned using Clustal Omega (www.ebi.ac.uk/Tools/msa/clustalo/). The alignment was generated in ESPript (espript.ibcp.fr/ESPript/cgi-bin/ESPript.cgi) using default settings. C-terminal sequences containing CTD domain are shown above. Strictly conserved residues are shown in white letters on black background. Residues >70% similarity are shown in black letters on grey background. NmeCas9 PAM interacting residues His1024 and Thr1027 that recognize NTS nucleotide G(-5)′ and A(-6)′ are denoted with arrows. AtCas9 mutation residues are boxed in black.

FIG. 17 shows engineering of AtCas9 PI domain capable of targeting broader PAM variants. A. Schematic representation of AtCas9 domain structure showing the positions of PAM-interacting (PI) mutations. BH, arginine-rich bridge helix; REC, recognition lobe; L1, L1 linker; L2, L2 linker; WED, Wedge domain; TOPO, Topoisomerase-homology domain; CTD, C-terminal domain; HNH, HNH domain; RuvC, RuvC domain; D, Aspartic acid; I, Isoleucine; S, Serine; G, Glycine; A, Alanine. B. Negative supercoil and linearized DNA of different PAM were cleaved by WT AtCas9 and two PI mutants, m4 and m5. Each PAM variant has at least two distinct spacer sequences for cleavage assays. Use Image Lab software to analyzed the percentage of cutting products and take the average value of each PAM variant for graphing. m4, D1089A; m5, D1089A S1091A G1092A. C. Sequence logo for the AtCas9 PI domain mutant m4 (D1089A) PAM as determined by linearized PAM library substrates cleavage assay. D. EMSA assay were performed with 5′ cy5-labeled oligonucleotides carrying WT PAM (CTAA, left) or MUT PAM (TATA, right), and dAtCas9 or dAtCas9-D1089A RNP complex titrating from 1.2 nM to 2.4 μM. KD±SD for dAtCas9 and dAtCas9-D1089A to WT PAM dsDNA, 26.16±12.05 nM and 44.43±15.39 nM; KD±SD for dAtCas9 and dAtCas9-D1089A to MUT PAM dsDNA, 109±7.1 nM and 27.15±13.6 nM. E. SpCas9 RNPs hardly bind to mut PAM (GTC) dsDNA compared with wt PAM (AGG) dsDNA. KD±SD for SpCas9 to WT PAM dsDNA, 300±10.57 nM; KD±SD for SpCas9 to MUT PAM dsDNA, ˜2.495e+18 nM.

FIG. 18 shows engineering of AtCas9 protein capable of improving activity and targeting broader PAM variants in mammalian cells. A. Shortlist of AtCas9 variants with increased activity. Different mutant versions of AtCas9 protein formed by replacing single or multiple amino acids of AtCas9 with the sequence of NmeCas9. B. Evaluation of the cleavage activity of AtCas9 variants against EGFP locus using gRNA-8 and gRNA-25. The edited genomic DNA was extracted, amplified and sanger sequencing. The sequencing results were analyzed by TIDE, and counting the fold change editing efficiency of mutants compared to wild-type AtCas9. C. C-to-T base editing efficiency of D1089A AtCas9 variants at four different loci (VEGFA, RUNXI, C-MYC and EGFP) harboring sixteen PAM combinations. Each dot represents the editing efficiency of one sgRNA. The sgRNAs targeting different PAMs are grouped with different color dots. The data was presented as mean value, and the gray shade are violin plots. n>=2 independent biological replicates. D. In vivo C-to-T base editing of negative supercoiled plasmids bearing sixteen PAM combinations in HEK293T cells. Plasmids encoding different mutants of AtCas9-CBE and sgRNA harboring spacer 36 were co-transfected with plasmid encoding PAM variants and amplicon sequencing was performed to calculate editing efficiency. Values and error bars reflect mean±s.d. of n=2 independent biological replicates.

FIG. 19 shows the mechanism of PAM-Cas9 recognition and DNA torque synergistically determine the activity of Cas9. When Cas9 searches its target site on the genome, the difference in DNA topology could affect the PAM recognition and DNA unwinding processes. Similar to other Cas orthologues, AtCas9 recognizes its PAM through the formation of hydrogen bond with C5 in target DNA. Besides this sequence-dependent recognition, the R sheet-loop-β sheet motif (shown as a loop) of AtCas9, closely docks into the major groove of dsDNA, serving as a second anchoring point to initiate the subsequent DNA unwinding. Underwinding DNA such as negative supercoil and Z-DNA are structurally different than B-form DNA and may have a stronger interaction with this motif, rendering longer bound time to mutated PAM. Furthermore, underwound DNA requires less energy to unwind the duplex, making it easier to form the R-loop. The combination of longer bound time to non-PAM and easier to unwind DNA duplex together leads to effective cleavage of non-PAM dsDNA in underwound topology.

FIG. 20 shows in vitro cleavage of diverse substrates. A-C showed that AtCas9 is capable of produce site-specific cleavage of ssDNA, ssRNA, and dsDNA. A. Fam-labeled single-stranded DNA substrate bearing 21A protospacer were incubated with AtCas9, tracrRNA and its cognate 21A-crRNA or a mismatched 21B-crRNA. Cleavage products were resolved on 12% PAGE gel. B, Cy3-labeled single-stranded RNA bearing 21A protospacer were incubated with AtCas9 RNP or AhCas9 RNP with its cognate 21A spacer. C. Comparison of AtCas9 activity against ssDNA, dsDNA and ssRNA substrates. 21A spacer were used in this experiments and cleavage were performed in buffer 16 for the indicated time points.

### DETAILED DESCRIPTION

### Terms and Definitions

The term “nucleic acid” refers to a polymeric form of nucleotides of any length, either ribonucleotides or deoxyribonucleotides. Thus, this term includes, but is not limited to, single- or double-stranded DNA or RNA, genomic DNA, cDNA, DNA-RNA hybrids, or a polymer comprising purine and pyrimidine bases or other natural, chemically or biochemically modified, non-natural, or derivatized nucleotide bases. “Oligonucleotide” generally refers to polynucleotides of between about 5 and about 100 nucleotides of single- or double-stranded DNA. However, for the purposes of this disclosure, there is no upper limit to the length of an oligonucleotide. Oligonucleotides are also known as “oligomers” or “oligos” and may be isolated from genes, or chemically synthesized by methods known in the art.

The terms “polypeptide”, “peptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” includes natural and/or unnatural or synthetic amino acids, including glycine and both the D or L optical isomers, and amino acid analogs and peptidomimetics.

By “hybridizable” or “complementary”, it is meant that a nucleic acid (e.g., RNA) comprises a sequence of nucleotides that enables it to non-covalently bind, i.e., form Watson-Crick base pairs and/or G/U base pairs, “Hybridize” to another nucleic acid in a sequence-specific, antiparallel, manner (i.e., a nucleic acid specifically binds to a complementary nucleic acid) under the appropriate in vitro and/or in vivo conditions of temperature and solution ionic strength. As is known in the art, standard Watson-Crick base-pairing includes: adenine (A) pairing with thymidine (T), adenine (A) pairing with uracil (U), and guanine (G) pairing with cytosine (C) [DNA, RNA]. Hybridization and washing conditions are well known and exemplified in Sambrook, J., Fritsch, E. F. and Maniatis, T. Molecular Cloning: A Laboratory Manual, Second Edition, Cold Spring Harbor Laboratory Press, Cold Spring Harbor (1989), particularly Chapter 11 and Table 11.1 therein; and Sambrook, J. and Russell, W., Molecular Cloning: A Laboratory Manual, Third Edition, Cold Spring Harbor Laboratory Press, Cold Spring Harbor (2001). The conditions of temperature and ionic strength determine the “stringency” of the hybridization. Hybridization requires that the two nucleic acids contain complementary sequences, although mismatches between bases are possible. The conditions appropriate for hybridization between two nucleic acids depend on the length of the nucleic acids and the degree of complementation, variables well known in the art. The greater the degree of complementation between two nucleotide sequences, the greater the value of the melting temperature (Tm) for hybrids of nucleic acids having those sequences. For hybridizations between nucleic acids with short stretches of complementarity (e.g., complementarity over 35 or less, 30 or less, 25 or less, 22 or less, 20 or less, or 18 or less nucleotides) the position of mismatches becomes important (see Sambrook et al., supra, 11.7-11.8). Typically, the length for a hybridizable nucleic acid is at least about 10 nucleotides. Illustrative minimum lengths for a hybridizable nucleic acid are: at least about 15 nucleotides; at least about 20 nucleotides; at least about 22 nucleotides; at least about 25 nucleotides; and at least about 30 nucleotides). Furthermore, the skilled artisan will recognize that the temperature and wash solution salt concentration may be adjusted as necessary according to factors such as length of the region of complementation and the degree of complementation.

“Binding” as used herein (e.g., with reference to an RNA-binding domain of a polypeptide) refers to a non-covalent interaction between macromolecules (e.g., between a protein and a nucleic acid). While in a state of non-covalent interaction, the macromolecules are said to be “associated” or “interacting” or “binding” (e.g., when a molecule X is said to interact with a molecule Y, it is meant the molecule X binds to molecule Y in a non-covalent manner). Binding interactions are generally characterized by a dissociation constant (Kd) of less than 10⁻⁶ M, less than 10⁻⁷ M, less than 10⁻⁸ M, less than 10⁻⁹ M, less than 10⁻¹⁰ M, less than 10⁻¹¹ M, less than 10⁻¹² M, less than 10⁻¹³ M, less than 10⁻¹⁴ M, or less than 10⁻¹⁵ M. “Affinity” refers to the strength of binding, increased binding affinity being correlated with a lower Kd.

A polynucleotide or polypeptide has a certain percent “sequence identity” to another polynucleotide or polypeptide, meaning that, when aligned, that percentage of bases or amino acids are the same, and in the same relative position, when comparing the two sequences. Sequence identity can be determined in a number of different manners. To determine sequence identity, sequences can be aligned using various methods and computer programs (e.g., BLAST, T-COFFEE, MUSCLE, MAFFT, etc.) See, e.g., Altschul et al. (1990), J. Mol. Bioi. 215:403-10.

The terms “non-naturally occurring” or “engineered” are used interchangeably and indicate the involvement of the hand of man. The terms, when referring to nucleic acid molecules or polypeptides mean that the nucleic acid molecule or the polypeptide is at least substantially free from at least one other component with which they are naturally associated in nature and as found in nature.

A “target nucleic acid” as used herein is a polynucleotide that comprises a “target sequence.” The terms “target sequence” refers to a nucleic acid sequence present in a target nucleic acid to which a guide sequence of a crRNA will bind, provided sufficient conditions for binding exist. Suitable DNA/RNA binding conditions include physiological conditions normally present in a cell. Other suitable DNA/RNA binding conditions (e.g., conditions in a cell-free system) are known in the art. The target sequence that is complementary to and hybridizes with the guide sequence in crRNA is referred to as the “target sequence (TS)” and the strand comprises TS is referred to as target strand. The sequence that is complementary to the “target sequence (TS)” (and is therefore not complementary to the guide sequence) is referred to as the “non-target sequence (NTS)” and the strand comprises NTS is referred to as non-target strand. When the target nucleic acid is single-strand DNA (ssDNA) or single-strand RNA (ssRNA), it refers to TS, that is, the guide sequence in crRNA is complementary to and can hybridizes with the ssDNA or the ssRNA.

By “cleavage” it is meant the breakage of the covalent backbone of a DNA or RNA molecule. Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. DNA cleavage can result in the production of either blunt ends or staggered ends. In certain embodiments, a complex comprising a guide RNA and a Cas9 protein is used for targeted double-stranded DNA cleavage. In certain embodiments, a complex comprising a guide RNA and a Cas9 protein is used for targeted cleavage of a single strand of a double-stranded target nucleic acid. In some embodiments, the cleavage results in the production of blunt ends.

By “Cas9 protein” it is meant a polypeptide that binds RNA and is targeted to a specific DNA sequence. A Cas9 protein as described herein is targeted to a specific nucleic acid sequence by the RNA (crRNA and tracrRNA) to which it is bound. The crRNA comprises a sequence that is complementary to a target sequence within the target nucleic acid, thus targeting the bound Cas9 protein to a specific location within the target nucleic acid (the target sequence). In the present disclosure, the target nucleic acid may be DNA or RNA, and may be single-strand or double-strand.

The term “mutant” or “variant” refers to a nucleic acid sequence that contains one or more additions, deletions, or substitutions of nucleic acid residues compared with the corresponding parental sequence, or a polypeptide sequence that contains one or more additions, deletions, or substitutions of amino acid residues compared to the corresponding parental sequence. When a mutant or a variant is mentioned, the numbering of mutation sites of the mutant or the variant is based on its corresponding parental sequence.

crRNA comprises both the guide sequence (also referred to as a “spacer”) and nucleotides stretch (“duplex-forming segment”) that contributes to the dsRNA duplex of the protein-binding segment. tracrRNA also comprises a nucleotide stretch (duplex-forming segment) that contribute to the dsRNA duplex of the protein-binding segment. In other words, the duplex-forming segment of a crRNA is complementary to and hybridizes with the duplex-forming segment of a tracrRNA to form the dsRNA duplex of the protein-binding domain. The guide sequence of crRNA acts as a targeting segment (a segment that hybridizes with the target sequence). Thus, a crRNA and a tracr-RNA (as a corresponding pair) hybridize to form a guide RNA. The exact sequence of a given tracrRNA) or crRNA molecule can be characteristic of the species in which the RNA molecules are found (or can be derived from such sequences, i.e., truncated, elongated, etc.).

In some embodiments, crRNA and tracrRNA are two separate RNA molecules. In other embodiments, crRNA and tracrRNA are existed in a single RNA molecule referred to herein as a “single guide RNA” or “sgRNA”. A sgRNA may comprises a crRNA which is fused to 5′ end of tracrRNA, tracrRNA is fused to 3′ end of crRNA. There may be a linker between crRNA and tracrRNA. The linker may be flexible, comprising G and A. An example of the linker is GAAA.

crRNA and tracrRNA can form a complex with a Cas9 protein (i.e., bind via non-covalent interactions). The crRNA provides target specificity of the complex by comprising a nucleotide sequence that is complementary to a target sequence of a target nucleic acid. The Cas9 protein of the complex provides the site-specific activity. In other words, the Cas9 protein is guided to a target sequence by virtue of its association with the protein-binding segment formed by hybridization of crRNA and tracrRNA. When the Cas9 protein has nuclease activity, site-specific cleavage of the target nucleic acid occurs where the complex is localized within the target nucleic acid, i.e., at a specific site (i.e., location) in the target nucleic acid determined by the base-pairing complementarity between the guide sequence of the crRNA and the target sequence of the target nucleic acid.

The term “in vitro” denotes outside, or external to cell, tissue, animal or human body, such as a cell-free system. The term “in vivo” denotes the situation in a cell, such as in a cell ex vivo, or in a cell within a tissue, animal or human body.

Unless otherwise indicated, the orientation of the nucleic acid sequence of the present disclosure is from 5′ to 3′.

In some embodiments, the methods of the present disclosure can be non-diagnostic and/or non-therapeutic.

It should be understood that this disclosure is not limited to particular embodiments described. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit, and the intervening range between the upper and lower limit of that range, is encompassed within the disclosure, unless the context clearly dictates otherwise. Where the stated range includes one or both of the limits, ranges excluding either or both of the limits included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described. All publications mentioned herein are to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a base” includes a plurality of bases and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The term “comprise”, “include”, “contain” and variations of these terms, such as comprising, comprises and comprised, are not intended to exclude further additions, components, integers or steps. These terms also encompass the meaning of “consist of” or “consisting of”.

the term “about” refers to a range equal to the particular value plus or minus twenty percent (+/−20%).

The term “and/or” refers to any one, any few or all of the elements connected by the term.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

Editing of Double-Stranded DNA with Relaxed PAM Requirement

The experimental examples revealed that PAM and DNA topology cooperate to determine the efficiency of DNA cleavage, and strikingly Cas9 is able to cleave PAM mutated dsDNA when the dsDNA topology is negative supercoil. Specifically, we identified a type II-C Cas9 from thermophile Alicyclobacillus tengchongensis, which exhibited distinct PAM preferences towards topologically different dsDNA. On linear dsDNA, AtCas9 has a relaxed PAM of CNNN and RNNA (R=A or G), covering 68% of all sequences. On physiologically negative supercoiled dsDNA, AtCas9 shows broader PAM preference of MNNN, TNNM and GNNA (M=C or A), covering 94% of all sequences.

It is contemplated that underwound dsDNA such as negative supercoils or Z-form DNA, have smaller torque and tend to promote R-loop formation, thereby facilitating the DNA unwinding process, and allowing broader PAM cleavage. This discovery, therefore, can be extrapolated to other Cas orthologues, such as SpCas9, NmeCas9 and Cas14a1.

Moreover, AtCas9 showed near PAM-less editing in E. coli, as the first naturally occurring Cas9 discovered to exhibit the near PAM-less editing ability. In mammalian cells, AtCas9 exhibited broad PAM preference to edit negative supercoiled plasmid and AtCas9-base editor showed high editing efficiency, up to 55% across several endogenous loci. These results uncover a novel thermophile Cas9 with relaxed PAM and reveal DNA torque as a new regulatory factor, highlighting the role of DNA topology in regulating Cas9 activity.

The present disclosure is related to a both sequence-specific and DNA topological structure-specific targeting method by CRISPR-Cas9 system. Cas9 mediated double-stranded DNA (dsDNA) cleavage is a signature of type II CRISPR/Cas systems. Of all studied Cas9 orthologues, DNA recognition is dependent on a protospacer adjacent motif (PAM) located next to the target dsDNA. The PAM complexity determines the available sites that can be edited by Cas9 system, thereby restricting the genome-editing ability to certain loci. Here, we identified two type II-C Cas9 from thermophiles Alicyclobacillus tengchongensis and A. hesperidum, both of which exhibited distinct PAM requirements towards topologically different dsDNA. On physiologically negative supercoiled dsDNA, both AtCas9 and AhCas9 displayed PAM-independent cleavage activity. By contrast, canonical PAM-dependent activity was observed on relaxed B-form DNA. Our analysis of various DNA topoisomers revealed that isomers with underwound DNA structure are crucial to enhance Cas9 activity when PAM is mutated. This DNA topology-guided PAM-independent cleavage is programmable and applicable to other Cas orthologues. Importantly, AtCas9 can mediate PAM-independent editing in Escherichia coli and is active in mammalian cells. These results reveal a new feature of Cas9 that enables both DNA sequence and topology specific target editing, highlighting the potential of exploiting the PAM-independent activity for editing different topoisomers.

In some embodiments, the present disclosure provides a method of targeting a target nucleic acid, the method comprising contacting the target nucleic acid with an engineered and non-naturally occurring CRISPR-Cas system comprising:
(i) a Cas9 protein derived from Alicyclobacillus sp. or a functional variant thereof, or a nucleic acid sequence encoding said Cas9 protein or the functional variant thereof;
(ii) a crRNA comprising a guide sequence that hybridizes to a target sequence in the target DNA, or a nucleic acid sequence encoding the crRNA; and
(iii) a tracrRNA that hybridizes with the crRNA;
wherein the crRNA and tracrRNA forms a complex with the Cas9 protein which causes binding of the Cas9 protein to the target sequence or cleavage of the target sequence.

The present disclosure also provides an engineered and non-naturally occurring CRISPR-Cas system used in above method.

The CRISPR-Cas9 system used in the present disclosure is both sequence-specific and DNA topological structure-specific. “Sequence-specific” refers to that the Cas9 protein can be targeted to the target sequence by hybridization of the guide sequence with the target sequence, thereby causing a binding of the Cas9 protein to the target sequence or a cleavage of the target sequence. “DNA topological structure-specific” refers to that the Cas9 protein can specify target nucleic acid with underwound topological structures and thus, distinguish from overwound or relax DNA topoisomers. when the target nucleic acid is underwound, the binding of the Cas9 protein to the target sequence or the cleavage of the target sequence is PAM-independent, that is, even if there is no PAM or a mutated PAM in the target nucleic acid, the Cas9 protein can still bind to or cleave the target sequence. When there is PAM in the target nucleic acid, the cleavage efficiency for an underwound substrate is higher than that for a non-underwound substrate.

### Alicyclobacillus Cas9 Protein and Variants and Cas9-Based Editing

The instant inventors have discovered new Cas9 proteins from Alicyclobacillus sp. that have more relaxed PAM requirements as compared to other known Cas proteins. In one embodiment, the present disclosure provides gene editing methods using these newly identified Cas proteins and their biological equivalents.

In one embodiment, the present disclosure provides a method for editing a target nucleic acid. In some embodiments, the method entails contacting the target nucleic acid with a CRISPR-Cas system that includes a Cas9 protein derived from Alicyclobacillus sp. or a functional variant thereof, and a guide RNA comprising a guide sequence that hybridizes to a target sequence in the target nucleic acid.

In some embodiments, the target sequence is adjacent to a protospacer adjacent motif (PAM). The PAM sequence of such a Cas9 protein may be CNNN and RNNA, wherein R is A or G, and each N is independently A, T, C, or G.

In some embodiments, the target nucleic acid has an underwound topology, and in such a situation, the PAM sequence requirement can be more relaxed (e.g., further include DNNB, where D is A, T or C, and B is T, C or G), or is even not required. As discussed later, the underwound topology may be natural in the target nucleic acid, or introduced by the present technology.

A natural underwound topology, for instance, can be found in a bulged double stranded DNA, or Z-DNA. In some embodiments, the bulged DNA has one or more consecutive unpaired bases within positions 1-10 from 3′ of the complementary sequence of the target sequence.

The Cas9 protein used in the present disclosure may be derived from Alicyclobacillus sp. A Cas9 protein derived from Alicyclobacillus sp. may have a naturally-existing sequence in a natural cell of Alicyclobacillus sp., i.e., a wild-type Cas9 protein derived from Alicyclobacillus sp. In some embodiments, the Cas9 protein derived from Alicyclobacillus sp. may be derived from Alicyclobacillus tengchongensis or Alicyclobacillus hesperidum, or Alicyclobacillus Sacchari. In some embodiments, the Cas9 protein derived from Alicyclobacillus sp. may be derived from Alicyclobacillus tengchongensis may have a sequence as set forth in SEQ ID NO: 84. In some embodiments, the Cas9 protein derived from Alicyclobacillus sp. may be derived from Alicyclobacillus hesperidum may have a sequence as set forth in SEQ ID NO: 85.

The Cas9 protein used in the present disclosure may be a functional variant of the Cas9 protein derived from Alicyclobacillus sp. The functional variant may have a sequence identity of no less than 70% with a Cas9 protein derived from Alicyclobacillus sp. and have at least one activity of a Cas9 protein derived from Alicyclobacillus sp. In some embodiments, the functional variant also may comprise insertion, substitution and/or deletion of one, two, three, four, five, six, seven, eight, nine, ten or more amino acids compared with a Cas9 protein derived from Alicyclobacillus sp. and have at least one activity of a Cas9 protein derived from Alicyclobacillus sp. The activities of a Cas9 protein include RuvC activity, HNH activity and PAM-Interacting (PI) activity, and the functional variant may have at least one of RuvC activity, HNH activity and PAM-Interacting (PI) activity, such as two or three of RuvC activity, HNH activity and PAM-Interacting (PI) activity. In some embodiments, the functional variant can form a complex with crRNA and tracrRNA and target the target sequence through the hybridization of guide sequence on crRNA with the target sequence and can PAM-independently target a underwound target nucleic acid. In some embodiments, the functional variant may have no or reduced PAM-Interacting activity compared with its parental Cas9 protein, but have RuvC activity and/or HNH activity. In some embodiments, the functional variant may have no or reduced RuvC activity and/or HNH activity compared with its parental Cas9 protein, but have PAM-Interacting activity. In some embodiments, the parental Cas9 protein is derived from Alicyclobacillus sp.

The functional variant may have a sequence identity of no less than 75%, no less than 80%, no less than 85%, no less than 90%, no less than 91%, no less than 92%, no less than 93%, no less than 94%, no less than 95%, no less than 96%, no less than 97%, no less than 98%, no less than 99%, no less than 99.5%, no less than 99.9% or no less than 100% with a Cas9 protein derived from Alicyclobacillus sp. In some embodiments, the functional variant may have a sequence identity of no less than 70%, no less than 75%, no less than 80%, no less than 85%, no less than 90%, no less than 91%, no less than 92%, no less than 93%, no less than 94%, no less than 95%, no less than 96%, no less than 97%, no less than 98%, no less than 99%, no less than 99.5%, no less than 99.9% or no less than 100% with a Cas9 protein derived from Alicyclobacillus tengchongensis or Alicyclobacillus hesperidum. The functional variant may have a sequence identity of no less than 70%, no less than 75%, no less than 80%, no less than 85%, no less than 90%, no less than 91%, no less than 92%, no less than 93%, no less than 94%, no less than 95%, no less than 96%, no less than 97%, no less than 98%, no less than 99% or no less than 100% with a Cas9 protein having a sequence set forth in SEQ ID NO:84 or SEQ ID NO:85. In some embodiments, the functional variant may be derived from Alicyclobacillus sp. In some embodiments, the functional variant may be derived from Alicyclobacillus tengchongensis or Alicyclobacillus hesperidum.

In some embodiments, the functional variant may comprise one, two, three, four, five, six, seven, eight, nine, ten or more amino acid insertions, substitutions and/or deletions in the RuvC domain and/or the HNH domain compared with its parental Cas9 protein such that the functional variant binds to but does not cleave the target nucleic acid. In some embodiments, the functional variant may comprise mutations of D8, H617A, N640A compared with the Cas9 protein derived from Alicyclobacillus sp. (such as SEQ ID NO:84 or SEQ ID NO:85). In some embodiments, the functional variant may comprise one, two, three, four, five, six, seven, eight, nine, ten or more amino acid insertions, substitutions and/or deletions in the PAM-Interacting (PI) domain compared with its parental Cas9 protein such that the Cas9 protein variant does not interact with the PAM. In some embodiments, the Cas9 protein variant may comprise mutations of D1089A, S1091A and G1092A compared with the Cas9 protein derived from Alicyclobacillus sp. (such as SEQ ID NO:84 or SEQ ID NO:85). In some embodiments, the Cas9 protein variant may comprise mutations of R1065A, T1094A, T1096A, D1089A, S1091A and G1092A compared with the Cas9 protein derived from Alicyclobacillus sp. (such as SEQ ID NO:84 or SEQ ID NO:85). Since the CRISPR-Cas9 system is capable of PAM-independent targeting a underwound target nucleic acid, a Cas9 protein variant which does not interact with the PAM can be used to target a underwound target nucleic acid.

In some embodiments, some functional variants are used to target a target nucleic acid in a PAM-independent way, regardless of the topology of the target nucleic acid. The inventor finds that some functional variants comprising mutations in the PAM-Interacting (PI) domain may cleavage a target nucleic acid with random sequence at the 3′ end of non-target sequence (NTS) even when the target nucleic acid is not a underwound target nucleic acid, such as a linear dsDNA, an open circle dsDNA, a positive supercoiled dsDNA or a B-DNA. In other words, some functional variants with mutations in the PI domain can target the target nucleic acid without interacting with PAM. Therefore, these variants can target the target nucleic acid without a specific PAM sequence, that is, there may be random sequence at the two ends (5′ end and/or 3′ end) of the target sequence in the target nucleic acid, and no specific PAM sequence (such as the PAM sequence of its parent protein) is needed. It can be also said that these variants have the PAM sequence of NNNNNNNN, where each N can independently be any one of A, T, C or G. For such functional variants, that target nucleic acid may be in any form, including a underwound dsDNA, a non-underwound dsDNA or a relaxed dsDNA, a ssDNA or a ssRNA. Such functional variants may comprise mutations in the PAM-Interacting (PI) domain compared with its parental Cas9 protein such that such that the functional variant can bind to or cleave a target sequence in a PAM-independent way.

The mutations may comprise one, two, three, four, five, six, seven, eight, nine, ten or more amino acid insertions, substitutions and/or deletions compared with its parental Cas9 protein. In some embodiments, the mutations may occur at one or more positions selected from the group consisting of E530, S531, L536, L602, D603, V604, T605, R1065, E1066, D1068, D1089, S1091, G1092, T1094, L1095, and T1096 of the Cas9 protein derived from Alicyclobacillus sp. (such as SEQ ID NO:84 or SEQ ID NO:85). In some embodiments, the mutations may occur at one or more positions selected from the group consisting of E530, S531, L536, L602, D603, V604, T605, R1065, E1066, D1068, D1089, S1091, G1092, T1094, L1095, and T1096 protein derived from Alicyclobacillus sp. (such as SEQ ID NO:84 or SEQ ID NO:85). In some embodiments, the mutations may select from the group consisting of E530A, S531R, L536T, L602I, D603N, V604L, T605G, R1065A, E1066K, D1068K, D1068R, D1089A, D1089E, S1091A, G1092A, T1094A, L1095A, and T1096A and any combination of them. In some embodiments, the mutations may be mutations of D1089A, S1091A and G1092A, mutations of E530A, S531R, L536T, L602I, D603N, V604L, T605G, R1065A, E1066K, D1068K, D1068R, D1089A, D1089E, S1091A, G1092A, T1094A, L1095A, and T1096A.

The mutations may comprise one, two, three, four, five, six, seven, eight, nine, ten or more amino acid insertions, substitutions and/or deletions compared with its parental Cas9 protein. In some embodiments, the mutations may occur at one or more positions selected from the group consisting of D1089, S1091, G1092, T1094, L1095, T1096 and R1065 of the Cas9 protein derived from Alicyclobacillus sp. (such as SEQ ID NO:84 or SEQ ID NO:85). In some embodiments, the mutations may occur at one or more positions selected from the group consisting of D1089, S1091, G1092, T1094, T1096 and R1065 of the Cas9 protein derived from Alicyclobacillus sp. (such as SEQ ID NO:84 or SEQ ID NO:85). In some embodiments, the mutations may select from the group consisting of D1089A, D1089E, S1091A, G1092A, T1094A, T1096A, R1065A and any combination of them. In some embodiments, the mutations may be mutations of D1089A, S1091A and G1092A, mutations of R1065A, T1094A, T1096A, D1089A, S1091A and G1092A, or mutation of T1096A. Example mutations are illustrated in Table A below.

TABLE A
Example Mutations in Alicyclobacillus sp Cas9

| Residue | Mutations |
|---|---|
| E530 | E530A, E530G, E530I, E530L, E530V |
| S531 | S531R, S531K |
| L536 | L536T, L536S |
| L602 | L602I, L602A, L602G, L602L, L602V |
| D603 | D603N, D603Q |
| V604 | V604L, V604A, V604G, V604I, V604V |
| T605 | T605G, T605A, T605I, T605L, T605V |
| R1065 | R1065A, R1065G, R1065I, R1065L, R1065V |
| E1066 | E1066K, E1066R |
| D1068 | D1068K, D1068R |
| D1089 | D1089A, D1089G, D1089I, D1089L, D1089V |
| D1089 | D1089E, D1089D |
| S1091 | S1091A, S1091G, S1091I, S1091L, S1091V |
| G1092 | G1092A, G1092G, G1092I, G1092L, G1092V |
| T1094 | T1094A, T1094G, T1094I, T1094L, T1094V |
| L1095 | L1095A, L1095G, L1095I, L1095L, L1095V |
| T1096 | T1096A, T1096G, T1096I, T1096L, T1096V |

Therefore, the present disclosure also provides a functional variant that comprise mutations in the PAM-Interacting (PI) domain compared with its parental Cas9 protein such that such that the functional variant can bind to or cleave a target sequence in a PAM-independent way, as described above. Also provided includes an isolated nucleic acid encoding said functional variant, an expression vector containing said nucleic acid, a host cell comprising said nucleic acid or expression vector, a system comprising said functional variant and corresponding crRNA and tracrRNA, a method of targeting a target nucleic acid using said system, and use of said functional variant.

The Cas9 protein or the functional variants thereof may comprise one or more nuclear localization signal (NLS) domains. The one or more one or more nuclear localization signal (NLS) domains may comprise at least two or more NLS domains. The one or more NLS domain(s) may be positioned at or near or in proximity to a terminus of the Cas9 protein or the functional variants thereof and if two or more NLSs, each of the two may be positioned at or near or in proximity to a terminus of the Cas9 protein or the functional variants thereof, or positioned at two terminuses of the Cas9 protein or the functional variants thereof respectively. In some embodiments, at least one nuclear localization signal (NLS) is attached to the nucleic acid sequences encoding the Cas9 protein or the functional variants thereof. In some embodiments, at least one or more C-terminal or N-terminal NLSs are attached (and hence nucleic acid molecule(s) coding for the Cpf1 effector protein can include coding for NLS(s) so that the expressed product has the NLS(s) attached or connected). In a preferred embodiment, a C-terminal NLS is attached for optimal expression and nuclear targeting in eukaryotic cells, preferably human cells.

In some embodiments, the nucleic acid sequence encoding the Cas9 protein or the functional variants thereof may be codon optimized for expression in particular cells, such as eukaryotic cells. The eukaryotic cells may be those of or derived from a particular organism, such as a mammal, including but not limited to human, mouse, rat, rabbit, dog, or non-human primate. In general, codon optimization refers to a process of modifying a nucleic acid sequence for enhanced expression in the host cells of interest by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence. Codon usage tables are readily available, for example, at the “Codon Usage Database”, and these tables can be adapted in a number of ways. See Nakamura, Y, et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, Pa.), are also available.

The term “target” or “targeting” in the context of that the Cas9 protein or the functional variants thereof target the target nucleic acid refers to the binding of the cas9 protein or the functional variants thereof to the target sequence or the cleavage of the target sequence by the cas9 protein. When a Cas9 protein variant that binds to but does not cleave the target nucleic acid is used, the term “target” or “targeting” in the context of that the Cas9 protein target the target nucleic acid refers to the binding of the cas9 protein to the target sequence without cleavage of the target sequence.

Guide RNAs are known in the art. A guide RNA may be a crRNA that is used together with a tracrRNA, a single guide RNA (sgRNA), or a prime editing guide RNA (pegRNA). crRNA and tracrRNA are known by those skilled in the art. crRNA comprises a guide sequence at 5′ end and a duplex-forming sequence at 3′ end. tracrRNA comprises a duplex-forming sequence at 5′ end. The duplex-forming sequence of the crRNA is complementary to and hybridizes with the duplex-forming segment of the tracrRNA to form a guide RNA, and the guide RNA and the Cas9 protein or the functional variants thereof form a complex. The guide sequence on crRNA hybridizes with the target sequence on the target nucleic acid, thereby causing the Cas9 protein or the functional variants thereof to bind to or cleave the target sequence.

The length of the guide sequence may be in the range of 20-30 nt, preferably 20-22 nt, 20-25 nt, 25-30 nt, such as 20, 21 nt, 22 nt, 23 nt, 24 nt, 25 nt, 26 nt, 27 nt, 28 nt, 29 nt or 30 nt.

The exact sequence of tracrRNA or crRNA molecule can be characteristic of the species in which the RNA molecules are found (or can be derived from such sequences, i.e., truncated, elongated, etc.). In some embodiments, the crRNA comprises a guide sequence flanked at its 3′ by a duplex-forming sequence and the duplex-forming sequence comprises a sequence selected form the group consisting of SEQ ID NO:73 and SEQ ID NO:74. In some embodiments, the tracrRNA comprises a sequence selected form the group consisting of SEQ ID NO:1 and SEQ ID NO: 70-72.

crRNA and tracrRNA may be two separate RNA molecules. In other embodiments, tracrRNA may be fused to 3′ end of crRNA to form a single RNA molecule referred to as single guide RNA (sgRNA). There may be a linker between crRNA and tracrRNA. The linker may comprise G and A which renders the linker flexible. An example of the linker is GAAA.

The Cas9 protein or the functional variants thereof may be provided in the form of a purified protein or in the form of a nucleic acid sequence encoding the Cas9 protein or the functional variants thereof. The nucleic acid encoding the Cas9 protein or the functional variants thereof may be included in a vector, such as a plasmid or a viral vector. The nucleic acid encoding the Cas9 protein or the functional variants thereof may be introduced into a cell, for example, by transfection, electroporation, liposomes, microinjection, and the like, so as to express the Cas9 protein or the functional variants thereof. The nucleic acid sequence encoding the cas9 protein or the functional variants thereof may also be integrated into the genome of a cell, so that the cell expresses the cas9 protein. Cells expressing the Cas9 protein or the functional variants thereof may only be used to provide recombinantly expressed Cas9 protein or the functional variants thereof and the cells may also contain a target nucleic acid so that when crRNA and tracrRNA are introduced into the cell, the target nucleic acid is brought into contact with the CRISPR-Cas system.

crRNA and tracrRNA may be provided in the form of an isolated RNA molecule or in the form of a nucleic acid sequence encoding the crRNA and/or tracrRNA. An isolated RNA molecule may be prepared by any in vitro transcription system known in the art. A vector, such as a plasmid or a viral vector, containing a nucleic acid sequence encoding crRNA or tracrRNA and a promoter can also be used to express crRNA or tracrRNA. The promoter may be operably linked to the nucleic acid sequence encoding crRNA or tracrRNA. The nucleic acid encoding the crRNA or the tracrRNA may be introduced into a cell contain a target nucleic acid, for example, by transfection, electroporation, liposomes, microinjection, and the like.

crRNA and tracrRNA may be expressed in two vectors, respectively, or be expressed in a single vector. crRNA and tracrRNA may be fused to a sgRNA. crRNA and tracrRNA may each have independent promoter and are expressed in a single vector, or a sgRNA may be expressed in a vector.

For an in vitro target nucleic acid, the CRISPR-Cas system may be provided by the purified Cas9 protein or the functional variants thereof and a duplex formed by isolated crRNA and tracrRNA, and the purified Cas9 protein or the functional variants thereof and the duplex formed by isolated crRNA and tracrRNA are brought into contact with the target nucleic acid.

For an in vivo target nucleic acid, such as a target nucleic acid within a cell, the CRISPR-Cas system may be provided by a vector expressing the Cas9 protein or the functional variants thereof and a vector expressing the crRNA and tracrRNA (such as a vector expressing the sgRNA), and the vector expressing the Cas9 protein or the functional variants thereof and the vector expressing the crRNA and tracrRNA are introduced into the cell. The Cas9 protein or the functional variants thereof and the crRNA and tracrRNA can be expressed in the same vector and the vector is introduced into the cell. For an in vivo target nucleic acid, such as a target nucleic acid within a cell, the CRISPR-Cas system may also be provided by a nucleic acid encoding the Cas9 protein or the functional variants thereof that is integrated into the genome of the cell and a vector expressing the crRNA and tracrRNA (such as a vector expressing the sgRNA), and the vector expressing the crRNA and tracrRNA is introduced into the cell.

### Editing of Underwound Double Stranded DNA

The present technology is also applicable to other Cas proteins. When the target double stranded DNA has an underwound topology, the PAM requirement for the corresponding Cas protein is relaxed. The underwound topology may be naturally occurring in a cell, or be generated with the present technology.

Many proteins (enzymes) are known in the art that can introduce negative topology to a target DNA. Non-limiting examples are provided in Table B below.

TABLE B
Example proteins capable of introducing underwound topology

| Protein | Function | Species |
|---|---|---|
| nonspecific E. | The dimeric protein | E. coli |
| coli heat- | introduces large | |
| unstable (HU) | deformations in DNA- | |
| protein | sharp bends, helical | |
| | untwisting, and | |
| | helical axis dislocation | |
| UvrD Helicase | acts on DNA repair | E. coli |
| Rep Helicase | DNA replication | E. coli |
| PcrA Helicase | DNA replication | Bacillus |
| | | stearothermophilus |
| Dda Helicase | replication initiation | bacteriophage T4 |
| RecQ Helicase | DNA repair | E. coli |
| eIF4A Helicase | RNA translation | Saccharomyces |
| | | cerevisiae |
| WRN Helicase | DNA Repair | Humans |
| NS3 Helicase | replication | Hepatitis C virus |
| TRCF (Mfd) | transcription-repair coupling | E. coli |
| Helicase | factor | |
| Ltag Helicase | reproduced | SV40 |
| E1 Helicase | replicating | human papillomavirus |
| Rep Helicase | replication, site-specific | Adeno-Associated |
| | integration, virion packaging | Virus (AAV) |
| DnaB Helicase | replication | E. coli |
| gp41 Helicase | DNA replication | bacteriophage T4 |
| T7gp4 Helicase | DNA replication | bacteriophage T7 |
| Rho Helicase | Transcription termination | E. coli |
| | factor | |
| DNA Helicase | roles in the initiation of | higher eukaryotes |
| B (HELB) | DNA replication and in the | |
| | DNA damage and replication | |
| | stress responses | |
| RecD Helicase | DNA repair | E. coli |
| RecBCD Helicase | DNA repair | E. coli |
| Pif1 Helicase | DNA replication, repair and | all eukaryotes and |
| | recombination | some bacteria |
| Rrm3 Helicase | DNA replication, repair and | is conserved from |
| | recombination | yeasts to humans |
| DNA gyrase | Topoisomerase | E. coli |
| A and B | | |

Such an enzyme can be used to introduce underwound topology to the target double stranded DNA. For instance, if the target DNA is cell free, the enzyme can be simply added to the DNA sample and reduces coiling of the target DNA generally.

For introduction of underwound topology more specifically at the target site, the enzyme may be coupled to the Cas protein or the guide RNA. In one example, the enzyme is just fused to the Cas protein.

In another example, the enzyme is coupled to the Cas protein indirectly, such as through a pair of orthogonal proteins, such as ligand/receptor or enzyme/substrate. For instance, the Cas protein can be fused or otherwise covalently connected to a ligand, and the enzyme is fused or otherwise covalently connected to a corresponding receptor.

Accordingly, one embodiment provides a fusion protein or a complex that includes a Cas protein and an enzyme capable of changing the topology of a double stranded DNA.

Also provided is method for editing a target nucleic acid, comprising contacting the target nucleic acid with a CRISPR-Cas system comprising a Cas protein, and a guide RNA comprising a guide sequence that hybridizes to a target sequence in the target nucleic acid, wherein the Cas protein or the guide RNAis covalently or non-covalently coupled to an enzyme capable of changing the topology of the target nucleic acid.

Likewise, provided is a method for editing a target nucleic acid, comprising contacting the target nucleic acid with a CRISPR-Cas system comprising a Cas protein with a corresponding protospacer adjacent motif (PAM) required for targeting a linear double stranded DNA, and a guide RNA comprising a guide sequence that hybridizes to a target sequence in the target nucleic acid, adjacent to a target PAM sequence, wherein the target sequence has an underwound topology and the target PAM sequence is not the corresponding PAM of the Cas protein. The underwound topology may be naturally occurring or introduced by an enzyme.

The term “Cas protein” or “clustered regularly interspaced short palindromic repeats (CRISPR)-associated (Cas) protein” refers to RNA-guided DNA endonuclease enzymes associated with the CRISPR (Clustered Regularly Interspaced Short Palindromic Repeats) adaptive immunity system in Streptococcus pyogenes, as well as other bacteria. Cas proteins include Cas9 proteins, Cas12a (Cpf1) proteins, Cas12b (formerly known as C2c1) proteins, Cas13 proteins and various engineered counterparts. Example Cas proteins include SpCas9, FnCas9, St1Cas9, St3Cas9, NmCas9, SaCas9, AsCpf1, LbCpf1, FnCpf1, VQR SpCas9, EQR SpCas9, VRER SpCas9, SpCas9-NG, xSpCas9, RHA FnCas9, KKH SaCas9, NmeCas9, StCas9, CjCas9, AsCpf1, FnCpf1, SsCpf1, PcCpf1, BpCpf1, CmtCpf1, LiCpf1, PmCpf1, Pb3310Cpf1, Pb4417Cpf1, BsCpf1, EeCpf1, BhCas12b, AkCas12b, EbCas12b, LsCas12b, RfCas13d, LwaCas13a, PspCas13b, PguCas13b, and RanCas13b.

Whether naturally occurring or introduced by the instant technology (e.g., with a fusion protein), the editing can be carried out with PAM sequences on the target DNA that does not qualify as the conventional PAM (or “canonical” PAM”) of the Cas protein. A canonical PAM for a Cas protein is one that is required when editing a linear or positively coiled DNA. For some of the commonly used Cas proteins, their canonical PAM sequences are listed in Table C.

TABLE C
Canonical PAM and Expanded PAM
under Underwound Conditions

| | | Canonical | Extended PAM in |
|---|---|---|---|
| Cas9 | Species | PAM | underwound DNA |
| SpCas9 | Streptococcus pyogenes | NGG | NAG, NGA |
| FnCas9 | Francisella novicida | NGG | NGA |
| SaCas9 | Staphylococcus aureus | NNGRRT | NNGRRV |
| NmeCas9 | Neisseria meningitidis | N₄GATT | N₄GCTT, N₄GTTT, |
| | | | N₄GACT, N₄GATA, |
| | | | N₄GTCT, N₄GACA |
| AsCas12a | Acidaminococcus sp. | TTTV | CTTV, TCTV, TTCV |
| AtCas9 | Alicyclobacillus | N₄CNNN, | N₄DNNB |
| | tenchongenous | N₄RNNA | |

Note:
R = A or G
D = A, T or G (not C)
B = T, C or G (not A)
V = A, C or G (not T)
N = A, T, C or G (any)

Therefore, in some embodiments, the Cas protein is SpCas9 and the corresponding PAM is NGG, wherein N is A, T, C or G. In some embodiments, the target PAM sequence is NAG or NGA.

In some embodiments, the Cas protein is FnCas9 and the corresponding PAM is NGG, wherein N is A, T, C or G. In some embodiments, the target PAM sequence is NGA.

In some embodiments, the Cas protein is SaCas9 and the corresponding PAM is NNGRRT, wherein each N is independently A, G, C or T, and each R is independently A or G. In some embodiments, the target PAM sequence is NNGRRV, wherein V is A, C or G.

In some embodiments, the Cas protein is NmeCas9 and the corresponding PAM is NNNNGATT, wherein each N is independently A, G, C or T. In some embodiments, the target PAM sequence is NNNNGCTT, NNNNGTTT, NNNNGACT, NNNNGATA, NNNNGTCT, or NNNNGACA.

In some embodiments, the Cas protein is AsCas12a and the corresponding PAM is TTTV, wherein V is A, C or G. In some embodiments, the target PAM sequence is CTTV, TCTV, or TTCV

In some embodiments, the Cas protein is AtCas9 and the corresponding PAM is CNNN and RNNA, wherein each N is independently A, T, C or G, and R is A G. In some embodiments, the target PAM sequence is any sequence other than CNNN and RNNA.

### Target Nucleic Acid and Method

The target nucleic acid is also referred to as substrate in the present disclosure. The target nucleic acid may be a DNA or an RNA and may be single-strand or double-strand. In some embodiments, the target nucleic acid may be a dsDNA or a ssDNA or a ssRNA.

In the case where the substrate is ssDNA or ssRNA, the guide sequence on crRNA hybridizes with the target sequence on the target nucleic acid, thereby causing the Cas9 protein to bind to or cleave the target sequence.

In the case where the substrate is dsDNA, the binding or cleavage mode of the CRISPR-Cas system of the present disclosure on the substrate varies according to the topology of the substrate. For a underwound dsDNA, such as a negatively supercoiled dsDNA, a bulged dsDNA or Z-DNA, the Cas9 protein of the present disclosure can PAM-independently bind to or cleave the substrate, that is, the Cas9 protein of the present disclosure can bind to or cleave a substrate that has no PAM or a mutated PAM near the target sequence. For a non-underwound dsDNA or a relaxed dsDNA, such as a linear dsDNA, open circle dsDNA, B-DNA or a positively supercoiled dsDNA, the binding of the Cas9 protein to the target sequence or the cleavage of the target sequence by the Cas9 protein is PAM-dependent, that is, the Cas9 protein only bind to or cleave a substrate that has a PAM at specific location near the target sequence.

However, even if there is PAM in the target nucleic acid, the cleavage efficiency for an underwound substrate is higher than that for a non-underwound substrate. In the present disclosure, cleavage efficiency or cleavage activity may be quantified by measuring the cleaved product intensity divided by cleaved product and uncleaved product on agarose gel.

PAMs are 2-8 base sequences adjacent the target sequence which can interact with the Cas9 protein. PAM is positioned at the 3′ end of non-target sequence (NTS). In the present disclosure, PAMs are 8 base sequences. For the Cas9 protein derived from Alicyclobacillus tengchongensis, such as a Cas9 protein having a sequence set forth in SEQ ID NO:84, the PAM sequence is NNNNCNNN and NNNNRNNA, wherein R is A or G and N is any one of A, C, T and G. For the Cas9 protein derived from Alicyclobacillus hesperidum, such as a Cas9 protein having a sequence set forth in SEQ ID NO:85, the PAM sequence is NNNNGNNA, wherein N is any one of A, C, T and G.

In the present disclosure, “PAM-independent” means that a Cas9 protein or the functional variants thereof target the target nucleic acid without interacting with PAM, i.e., target the target nucleic acid without a specific PAM sequence. In these cases, there may be random sequence at the two ends (5′ end and/or 3′ end) of the target sequence in the target nucleic acid, and no specific PAM sequence (such as a PAM sequence of a wild-type Cas9 protein or a PAM of the parental protein of a functional variant) is needed. It can be also said that the PAM sequence is NNNNNNNN, where each N can independently be any one of A, T, C or G. Therefore, in a PAM-independent targeting, the complementary sequence of the target sequence can be flanked or not flanked at its 3′ by a specific PAM sequence (such as a PAM sequence of a wild-type Cas9 protein in the case that the Cas9 protein is wild-type, or a PAM of the parental protein in the case that the Cas9 protein is a functional variant of the parental protein).

In the present disclosure, in the case of a PAM-dependent cleavage, the Cas9 protein or the functional variants thereof mediates a cleavage 3 bp upstream of the PAM. In the case of a PAM-independent cleavage, the Cas9 protein or the functional variants thereof mediates a cleavage 3 bp upstream of 5′ end of the target sequence. The cleavage mediates a double-strand break in the case where the substrate is dsDNA and the cleavage results in the cleavage of the single strand in the case where the substrate is ssDNA or ssRNA.

The term “underwound” used herein refers to DNA structures have a torque lower than the relaxed B-form DNA topoisomers. Naturally existing underwound DNA topoisomers includes negative supercoil, Z-DNA, bulged DNA and other. In general, underwound DNA usually locates behind a transcriptional active gene locus.

The term “supercoil” is defined as the physical state of a polynucleotide in which one strand of the polynucleotide is underwound or overwound in relation to other strands of the polynucleotide.

The term “negative supercoiled” is the left-handed coiling of DNA and thus winding occurs in the counterclockwise direction. It is also known as the “underwinding” of DNA. Prokaryotic cell genome generally exists in the form of a negative supercoil. Plasmid is generally a negative supercoil. Eukaryotic cells usually have negative supercoils in the dynamic process of transcription. For example, the downstream of transcribed gene will produce positive supercoils, while the upstream will produce negative supercoils.

As used herein, “bulged DNA” or “bulged oligonucleotides” refers to a double-stranded DNA with an unpaired base. A region of the unpaired base (or a region of non-complementarity due to the unpaired base) in the double stranded is referred to as a bulge. A bulge may be flanked on both sides by double-stranded DNA sequence. In some embodiments, a bulged DNA may be a double-stranded DNA with one or more consecutive unpaired bases, such as one, two, three, four, five, six, seven, eight, nine, ten or more consecutive unpaired bases. The unpaired base may be formed due to a base pair mismatch on the duplex. In some embodiments, the bulge is located in the region of target sequence in the target nucleic acid. In some embodiments, the target sequence (TS) hybridizes to the guide sequence and the non-target sequence (NTS) has two or more unpaired bases mismatched with the corresponding nucleotides in TS. In some embodiments, the unpaired bases are located at positions 1-2, 1-3, 1-4, 1-5, 1-6, 1-7, 1-8, 1-9, 1-10, 1-11, 1-12, 1-13, 1-14, 1-15, 1-16, 1-17, 1-18, 1-19, 1-20, 1-21, 1-22, 1-23, 1-24 or 1-25 from the 3′end of NTS. In eukaryotic cell, bulged DNA is transiently present when a mismatch is formed during DNA replication or DNA repair process.

As used herein, the term “Z-DNA” is also referred to as Z form DNA and refers to a left-handed conformation of the DNA double helix or RNA stem loop structures. Such DNA helices wind to the left in a zigzag pattern (as opposed to the right, like the more commonly found B-DNA form). In eukaryotic cell, Z-DNA has been demonstrated widely distributed in genome. One example is the promoter region of c-MYC gene, part of the sequence undergoes Z-form topological structure.

As used herein, the term “B-DNA” is also referred to as B form DNA and is the structure that non-supercoiled DNA typically adopts under physiological conditions. It consists of the classic Watson-Crick structure wherein there is a spacing of around 0.34 nanometers per base-pair, and about 10.5 base-pairs per turn. Eukaryotic cell genome is usually a relaxed B form DNA.

The term “positive supercoiled” is the right-handed, coiling of DNA and thus winding occurs in the clockwise direction. In both prokaryotic and eukaryotic system, positive supercoil is usually present downstream of DNA replication or transcription complex.

The term “open circle” DNA is a double-stranded circular DNA molecule that has been nicked in one of the strands to allow the release of any superhelical turns present in the molecule. The open circular form migrates more slowly during gel electrophoresis than a covalently closed circular, or supercoiled, molecule of the same size due to the associated differences in conformation, or shape, of the molecules.

The term “linear” dsDNA is a double-stranded DNA which is linear in shape, and contains terminal ends.

In some embodiments, it is useful to express all components of the system of the present disclosure (such as Cas9 protein or functional variant thereof, and crRNA and tracrRNA) in a host cell. A “host cell” can originate from any organism. Examples of host cells include, but are not limited to: a prokaryotic cell, a eukaryotic cell, a bacterial cell, an archaeal cell, a cell of a single-cell eukaryotic organism, a protozoa cell, a cell from a plant.

The target nucleic acid may be in vitro or in vivo. In some embodiments, the target nucleic acid is within a cell, such as a prokaryotic cell or a eukaryotic cell. The prokaryotic cell may be E coli. The eukaryotic cell may be a mammalian cell or a human cell. When the target nucleic acid is in vivo, the method of the present disclosure includes introducing the system of the present disclosure into the cell comprising the target nucleic acid.

Methods of introducing polynucleotides (e.g., an expression vector) into cells are known in the art and are typically selected based on the kind of the cell. Such methods include, for example, viral or bacteriophage infection, transfection, conjugation, electroporation, calcium phosphate precipitation, polyethyleneimine-mediated transfection, DEAE-dextran mediated transfection, protoplast fusion, lipofection, liposome-mediated transfection, particle gun technology, direct microinjection, and nanoparticle-mediated delivery. The contact between the CRISPR-Cas system of the present disclosure and the target nucleic acid may be performed in presence of divalent cation, preferably Mg², Ca²⁺ or Mn²⁺. The concentration of Mg²⁺ may be in a range from about 1 mM to about 50 mM, preferably in a range from about 2 to about 20 mM, preferably in a range from about 5 to about 10 mM, more preferably about 10 mM.

The contact between the CRISPR-Cas system of the present disclosure and the target nucleic acid may be performed at a temperature in a range from about 37° C. to about 65° C., preferably in a range from about 40° C. to about 60° C., preferably in a range from about 45° C. to about 55° C., preferably in a range from about 50° C. to about 55° C., more preferably of about 10 mM.

The contact between the CRISPR-Cas system of the present disclosure and the target nucleic acid may be performed at pH in a range from about 4 to about 9, preferably in a range from about 5 to about 8, preferably in a range from about 6 to about 8, preferably in a range from about 7 to about 8, more preferably of about 8.

In some embodiments, the contact between the CRISPR-Cas system of the present disclosure and the target nucleic acid is performed at a temperature of about 55° C. and pH of about 8.

The instant technology, including the newly discovered Cas proteins and their functional variants, fusion proteins and complexes, and editing of underwound DNA, can be applied not only in the conventional CRISPR-Cas editing system, but also in base editing and prime editing.

A base editor (BE) integrates the CRISPR/Cas system with the APOBEC (apolipoprotein B mRNA editing enzyme, catalytic polypeptide-like) AID (activation-induced cytidine deaminase) family. Through the fusion with the Cas9 nickase (nCas9) or a catalytically dead Cpf1 (dCpf1 also known as dCas12a), the nucleobase deaminase activity of APOBEC/AID family members can be purposely directed to the target bases in the genome and to catalyze base substitutions.

The term “nucleobase deaminase” as used herein, refers to a group of enzymes that catalyze the hydrolytic deamination of nucleobases such as cytidine, deoxycytidine, adenosine and deoxyadenosine. Non-limiting examples of nucleobase deaminases include cytidine deaminases and adenosine deaminases.

“Cytidine deaminase” refers to enzymes that catalyze the irreversible hydrolytic deamination of cytidine and deoxycytidine to uridine and deoxyuridine, respectively. Cytidine deaminases maintain the cellular pyrimidine pool. A family of cytidine deaminases is APOBEC (“apolipoprotein B mRNA editing enzyme, catalytic polypeptide-like”). Members of this family are C-to-U editing enzymes. Some APOBEC family members have two domains, one domain of APOBEC like proteins is the catalytic domain, while the other domain is a pseudocatalytic domain. More specifically, the catalytic domain is a zinc dependent cytidine deaminase domain and is important for cytidine deamination. RNA editing by APOBEC-1 requires homodimerisation and this complex interacts with RNA binding proteins to form the editosome.

Non-limiting examples of APOBEC proteins include APOBEC1, APOBEC2, APOBEC3A, APOBEC3B, APOBEC3C, APOBEC3D, APOBEC3F, APOBEC3G, APOBEC3H, APOBEC4, and activation-induced (cytidine) deaminase (AID).

Various mutants of the APOBEC proteins are also known that have bring about different editing characteristics for base editors. For instance, for human APOBEC3A, certain mutants (e.g., W98Y, Y130F, Y132D, W104A, D131Y and P134Y) even outperform the wildtype human APOBEC3A in terms of editing efficiency or editing window. Accordingly, the term APOBEC and each of its family member also encompasses variants and mutants that have certain level (e.g., 70%, 75%, 80%, 85%, 90%, 95%, 98%, 99%) of sequence identity to the corresponding wildtype APOBEC protein or the catalytic domain and retain the cytidine deaminating activity. The variants and mutants can be derived with amino acid additions, deletions and/or substitutions. Such substitutions, in some embodiments, are conservative substitutions.

“Adenosine deaminase”, also known as adenosine aminohydrolase, or ADA, is an enzyme (EC 3.5.4.4) involved in purine metabolism. It is needed for the breakdown of adenosine from food and for the turnover of nucleic acids in tissues.

Non-limiting examples of adenosine deaminases include tRNA-specific adenosine deaminase (TadA), adenosine deaminase tRNA specific 1 (ADAT1), adenosine deaminase tRNA specific 2 (ADAT2), adenosine deaminase tRNA specific 3 (ADAT3), adenosine deaminase RNA specific B1 (ADARB1), adenosine deaminase RNA specific B2 (ADARB2), adenosine monophosphate deaminase 1 (AMPD1), adenosine monophosphate deaminase 2 (AMPD2), adenosine monophosphate deaminase 3 (AMPD3), adenosine deaminase (ADA), adenosine deaminase 2 (ADA2), adenosine deaminase like (ADAL), adenosine deaminase domain containing 1 (ADAD1), adenosine deaminase domain containing 2 (ADAD2), adenosine deaminase RNA specific (ADAR) and adenosine deaminase RNA specific B1 (ADARB1).

Prime editing is a genome editing technology by which the genome of living organisms may be modified. Prime editing directly writes new genetic information into a targeted DNA site. It uses a fusion protein, consisting of a catalytically impaired endonuclease (e.g., Cas9) fused to an engineered reverse transcriptase enzyme, and a prime editing guide RNA (pegRNA), capable of identifying the target site and providing the new genetic information to replace the target DNA nucleotides. Prime editing mediates targeted insertions, deletions, and base-to-base conversions without the need for double strand breaks (DSBs) or donor DNA templates.

The pegRNA is capable of identifying the target nucleotide sequence to be edited, and encodes new genetic information that replaces the targeted sequence. The pegRNA consists of an extended single guide RNA (sgRNA) containing a primer binding site (PBS) and a reverse transcriptase (RT) template sequence. During genome editing, the primer binding site allows the 3′ end of the nicked DNA strand to hybridize to the pegRNA, while the RT template serves as a template for the synthesis of edited genetic information.

The fusion protein, in some embodiments, includes a nickase fused to a reverse transcriptase. An example nickase is Cas9 H840A. The Cas9 enzyme contains two nuclease domains that can cleave DNA sequences, a RuvC domain that cleaves the non-target strand and a HNH domain that cleaves the target strand. The introduction of a H840A substitution in Cas9, through which the histidine residue at 840 is replaced by an alanine, inactivates the HNH domain. With only the RuvC functioning domain, the catalytically impaired Cas9 introduces a single strand nick, hence a nickase.

Non-limiting examples of reverse-transcriptases include human immunodeficiency virus (HIV) reverse-transcriptase, moloney murine leukemia virus (M-MLV) reverse-transcriptase and avian myeloblastosis virus (AMV) reverse-transcriptase.

In some embodiments, the prime editing system further includes a single guide RNA (sgRNA) that directs the Cas9 H840A nickase portion of the fusion protein to nick the non-edited DNA strand.

### EXAMPLES

### Example 1. Distinct PAM Requirements for Thermostable Cas9 Orthologues

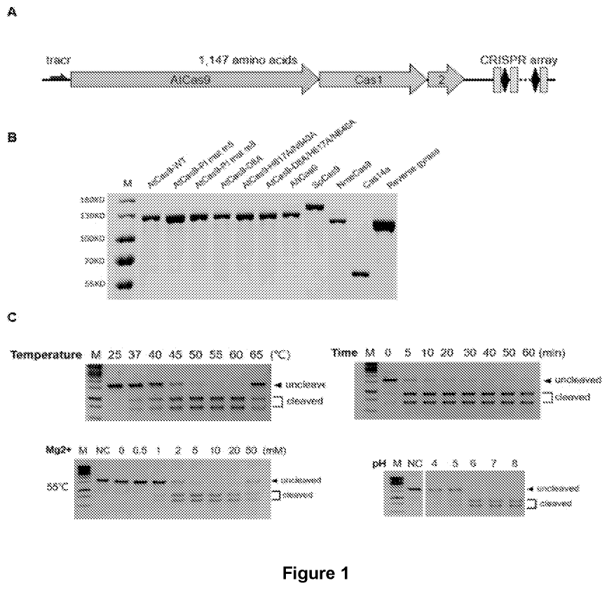

The thermo-acidophilic bacterium, Alicyclobacillus tengchongensis, originally isolated from the sediment in a hot spring, can grow in a wide temperature range from 30° C. to 65° C. Analyzing the whole-genome sequence, we found that A. tengchongensis carries an intact CRISPR locus including three Cas genes cas9, cas1, cas2 and the adjacent CRISPR array (FIG. 1A). We purified AtCas9 (GenBank Accession No. WP_058095017) and performed an initial biochemical analysis using one of the original spacers from the CRISPR locus (FIG. 1B-C). Using PCR derived dsDNA as the substrates, we found that AtCas9 is a Mg²⁺-dependent endonuclease that cleaves target DNA at a wide range of pH and temperature, from pH 5-8 and from 37° C.-65° C., respectively (optimal activity at pH 6-8 and 55-60° C.) (FIG. 1C).

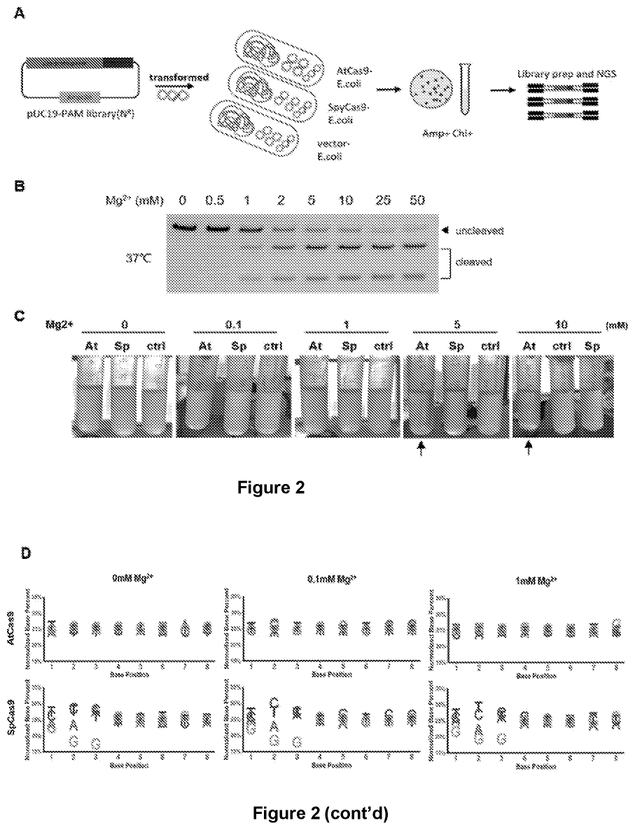

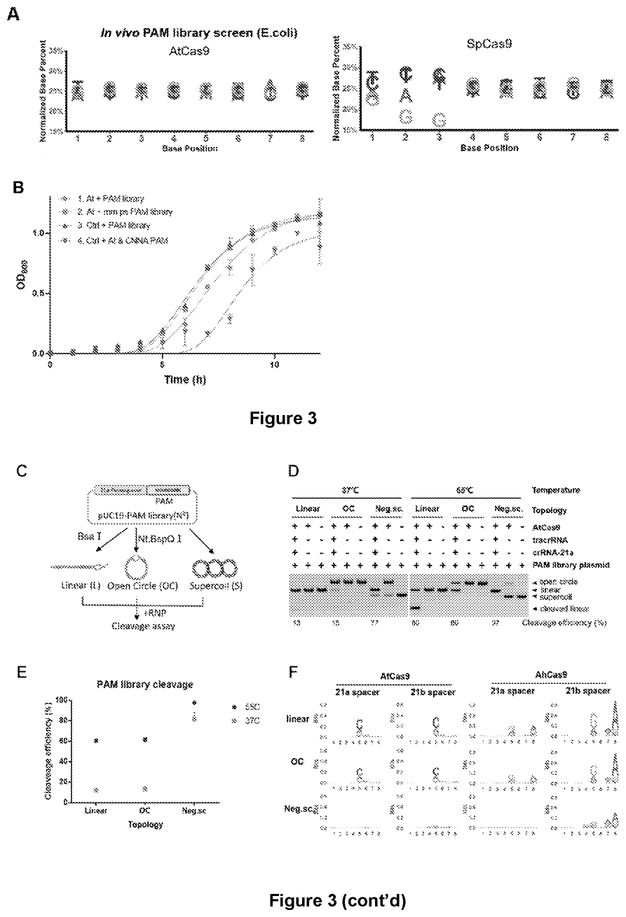

To identify the PAM for AtCas9, we constructed a PAM plasmid library containing 8 base-pair randomized nucleotides adjacent to the protospacer 21a, and performed transformation assays in E. coli harboring AtCas9 locus (FIG. 2A). To our surprise, AtCas9 exhibited no PAM preference whereas the positive control SpCas9 showed a classic NGG PAM (FIG. 3A). To rule out the possibility that the activity of Atcas9 is compromised at 37° C. in E. coli, we first tested different ion metals and found that increasing magnesium concentration enhanced cleavage activity at 37° C. (FIG. 2B). Similar to in vitro cleavage results, supplementation of magnesium in E. coli cultures markedly improved the cleavage of PAM library plasmid (FIG. 2C), suggesting that AtCas9 is functional in the prokaryotic system. Deep sequencing of E. coli culture supplemented with magnesium further confirmed that AtCas9 had no PAM preference towards plasmid library whereas SpCas9 preferred a NGG PAM (FIG. 2D).

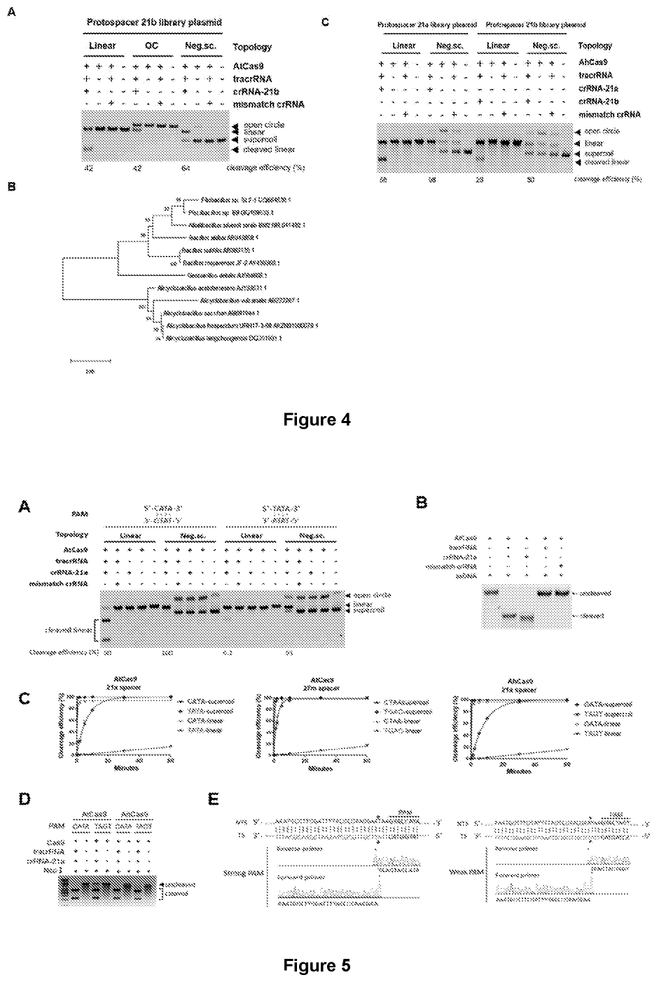

To rule out the possibility that AtCas9 is not active in mesophilic E. coli, we closely monitored cell growth as cleaved plasmid cannot replicate and cells gradually lost antibiotic expression and die in the selection medium. Compared to control cells that either don't express AtCas9 (FIG. 3B, positive triangle) or have a mismatched plasmid (FIG. 3B, squares), cells transformed with the matched PAM library plasmid exhibited slower cell growth (FIG. 3B, circles). In addition, co-transformation of AtCas9 locus and a known PAM substrate exhibited severe delay in cell growth (FIG. 3B, inverted triangle), strongly suggesting that AtCas9 is active and functions to cleave plasmid in E. coli. Since in vivo negative screen did not identify PAM preference, we turned to in vitro positive screen by direct sequencing of cleaved plasmids. We first treated PAM library plasmid with restriction enzymes BsaI or Nt.BspQI to generate linear or open circle DNA isomers for comparison with negative supercoiled plasmid isomer (FIG. 3C). Three topoisomers were incubated with AtCas9 in the presence of matched crRNA and tracrRNA and digested products were analyzed on 0.8% agarose gel (FIG. 3D). Interestingly, up to 97% supercoiled PAM library isomers were cleaved whereas only 60% linear or open circle isomers were digested at AtCas9's optimal temperature (FIGS. 3D and 3E). Similar trends were observed when a different spacer and its PAM library substrate were used (FIG. 4A). The near complete cleavage of negative supercoiled PAM library explains why the PAM screen using supercoiled plasmid as cleavage substrates in E. coli failed to identify any PAM preference. Noted that linear or open circle PAM library substrates displayed ˜60% cleavage, indicating that AtCas9 has a very relax PAM preference against these topoisomers as 60% of 65536 (4\hat{ }8) PAM combinations can be recognized and cleaved. To map the exact PAM preference, we sequenced the digested fragments. Using two different spacers with their respective protospacer substrates, we found AtCas9 has a preference of C or A or G at position 5 when the substrates are linear or open circle dsDNA (FIG. 3F left). In contrast, the preference is completely abolished when AtCas9 was incubated with negative supercoiled dsDNA (FIG. 3F left).

We next tested whether DNA topology guided near-PAMless cleavage is shared by other Cas9 orthologues. Phylogenetic analysis based on 16S rRNA sequences indicated that A. hesperidum shares 95% similarity to A. tenchongenous (FIG. 4B). We then purified AhCas9 protein and performed in vitro PAM identification towards different DNA topoisomers. Similar to AtCas9, AhCas9 exhibited a PAM preference of NNNNGNNA towards linear and open circle dsDNA, but no preference towards negative supercoiled DNA (FIG. 3F right and 4C). Collectively, these results suggest that DNA topology plays an important role in regulating PAM preference of AtCas9 and AhCas9.

### Example 2. PAM-Independent Cleavage is Programmable and Site Specific

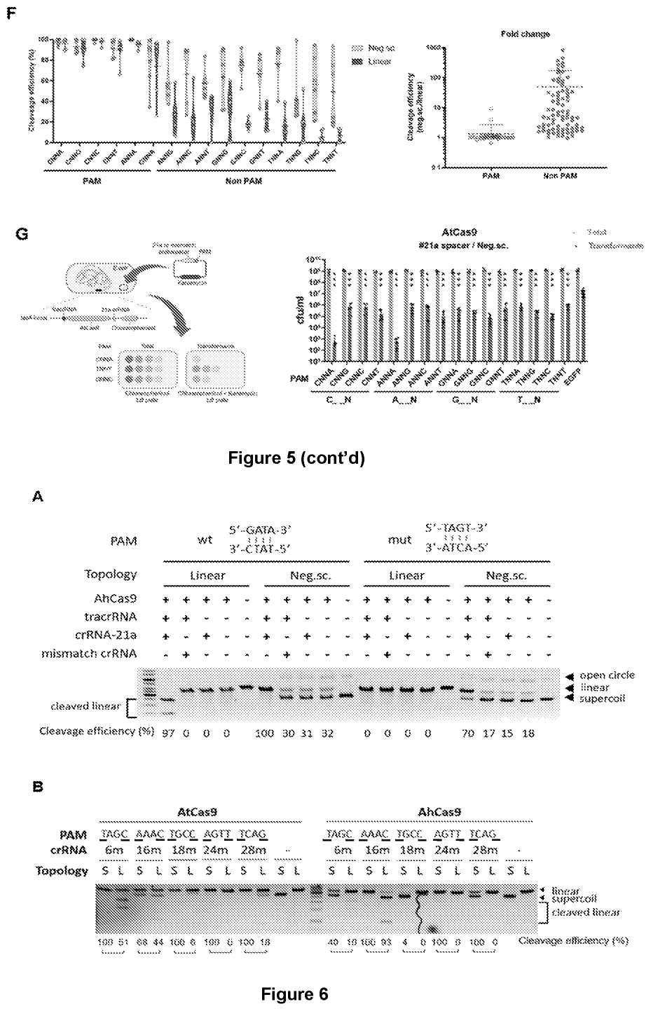

Compared with other Cas9 orthologues, AtCas9 has the most relaxed PAM with a preference of C, A, or G at position 5 when the substrate is linear or open circle dsDNA (FIG. 3F). Mutating PAM from CNNA to TNNA for AtCas9 or from GNNA to TNNT for AhCas9 completely abolished cleavage activity against linear dsDNA, confirming the requirement of PAM recognition and binding to initiate R-loop formation and subsequent cleavage (FIGS. 5A and 6A). In contrast, up to 91% cleavage of negative supercoiled dsDNA was observed when PAM was mutated (FIGS. 5A and 6A). Similar to other Cas9 orthologues, the cleavage requires the presence of tracrRNA and a matched crRNA (FIGS. 5A and 6A). To rule out the possibility that the cleavage of PAM mutant dsDNA is a consequence of single-stranded DNA (ssDNA) activity, we incubated AtCas9 RNP with complementary ssDNA. Consistent with type II-C Cas9 orthologues, AtCas9 is able to cleave ssDNA without the need of tracrRNA (FIG. 5B), thereby ruling out the possibility that negative supercoil induced cleavage of PAM mutant dsDNA is a consequence of Cas9 activity against ssDNA, whose cleavage doesn't require PAM binding.

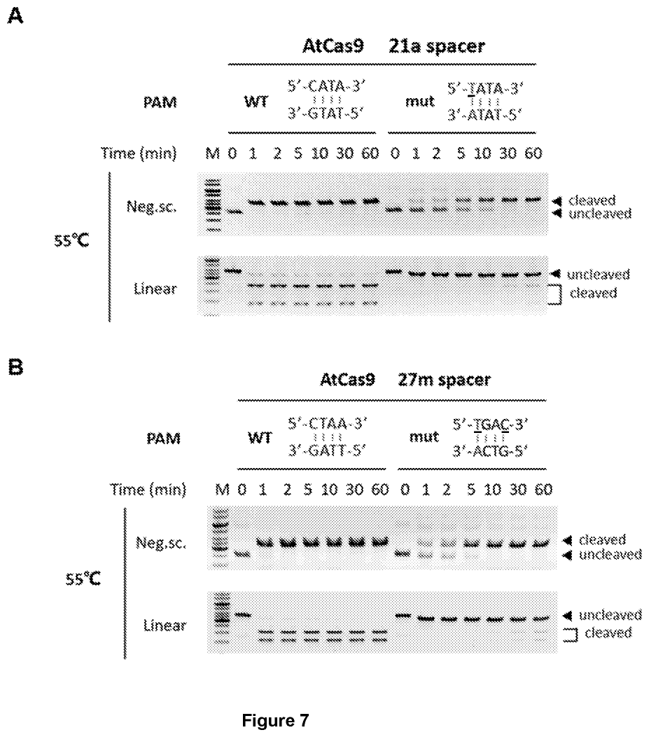

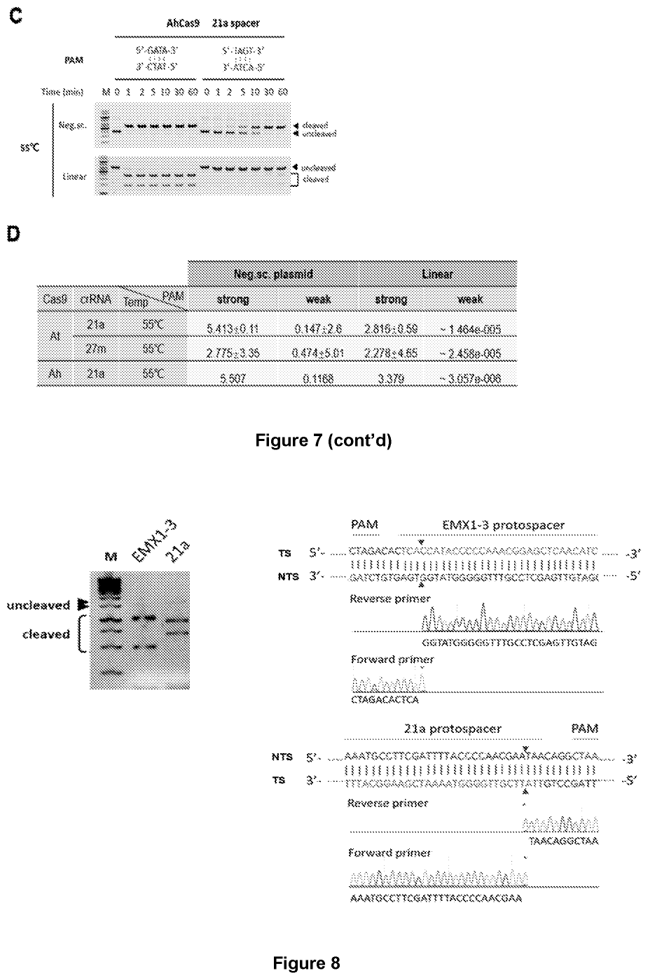

Kinetic analysis using two different spacers showed that AtCas9 and AhCas9 had robust cleavage activities against negative supercoiled DNA when PAM is mutated (FIG. 5C and FIG. 7A-D), suggesting that DNA supercoiling is preferred by these Cas9 proteins and is able to initiate DNA cleavage independent of PAM. Next, to determine whether the cleavage is site-specific, plasmids containing WT PAM or mutated PAM were cleaved in vitro with AtCas9 RNP or AhCas9 RNP, and the cleaved products were digested with a restriction enzyme NcoI (FIG. 5D). Gel analysis and sanger sequencing of digested product showed that regardless of WT or mutated PAM, AtCas9 and AhCas9 created a blunt end double stranded break at 3 bp upstream of PAM (FIGS. 5D and 5E). Similar results were observed when using linear DNA as substrates (FIG. 8). In contrast, Cas9 alone, exhibits non-specific cleavage (FIG. 5D), which may be caused by its nickase activity. Together, these data suggest that AtCas9 and AhCas9 can be programmed to cleave negative supercoiled DNA with mutated PAM sequence. We next investigated whether PAM-independent cleavage is programmable. We tested additional spacers and their corresponding DNA substrates in which PAMs were mutated. Linear PAM mutants showed little cleavage whereas their supercoiled topoisomers exhibited up to 100% cleavage by AtCas9 and AhCas9 (FIG. 6B), indicating that PAM-independent cleavage against supercoiling DNA is programmable for these two enzymes.

Our deep sequencing suggested that AtCas9 has a broad PAM preference based on two sgRNAs. To determine whether the PAM preference is shared by other sgRNAs, we tested up to 82 gRNAs with sixteen PAM combinations differing in position 5 and position 8. 50 nM in vitro transcribed gRNAs complexed with equal molar AtCas9 were incubated for 30 min at 55° C. with their corresponding substrates (3 nM) presented either as linear or negative supercoil. Cleavage efficiency was measured by quantifying the fraction of cleavage on the agarose gel. Taken all 82 gRNAs covering 16 PAM combinations, AtCas9 has a median efficiency of 60.1% when the dsDNA substrate is negative supercoil, compared with 16.2% in linear isomer (FIG. 5F). When presented with linear dsDNA (B-form), AtCas9 showed a canonical PAM CNNN, RNNA (R=A or G), with an average efficiency of 91.7% of 34 gRNAs tested (FIG. 2E). Non-PAM combinations such as RNNB (B=non A, R=A/G) and TNNN exhibited a median efficiency of 20% in linear dsDNA, indicating these sequences are less likely to be recognized by AtCas9 when presented as B-form DNA. When incubated with negative supercoiled dsDNA, PAM preferences extended to almost all non-PAM combinations except TNNG (FIG. 5F), with a median efficiency of 80%. Direct comparison of two topoisomers of each gRNAs, we found in non-PAM substrates, negative supercoil greatly enhance cleavage efficiency by an average of 50-fold over linear isomer, while a minor 1.3-fold increase of negative supercoil over linear isomer in PAM containing substrates (FIG. 5F right). Taken all 82 gRNAs covering sixteen PAM combinations and calculated the cleavage efficiency ratio of negative supercoil to linear DNA, the mean value of the fold change between canonical PAM and non-canonical PAM cleavage activity increased from 1.3 to 49.3 (FIG. 5F right). Together, our data strongly suggested AtCas9 has a wide range of PAM preferences with different cleavage activities and negative supercoil substrate is able to further enhance AtCas9 activities in PAM and non PAM cases.

Biochemical analysis showed that DNA topology helps facilitate the near PAMless cleavage of AtCas9. To determine whether this holds the case in vivo, we tested the editing of 16 PAM combinations in E. coli and in HEK293T respectively. A minimal AtCas9 locus including Cas9, tracrRNA and a repeat-spacer array was introduced into the lacA locus in E. coli (FIG. 5G left). We then transformed plasmids encoding a matching protospacer with 16 PAM combinations or a mismatch control. Comparing with non-targeting control (EGFP), all 16 PAM combinations showed at least 100-fold decrease in colony formation, suggesting AtCas9 is able to cleave supercoiled plasmid in a PAMless fashion (FIG. 5G).

### Example 3. DNA Unwinding Guided by its Topological Structure Facilitates dsDNA Cleavage

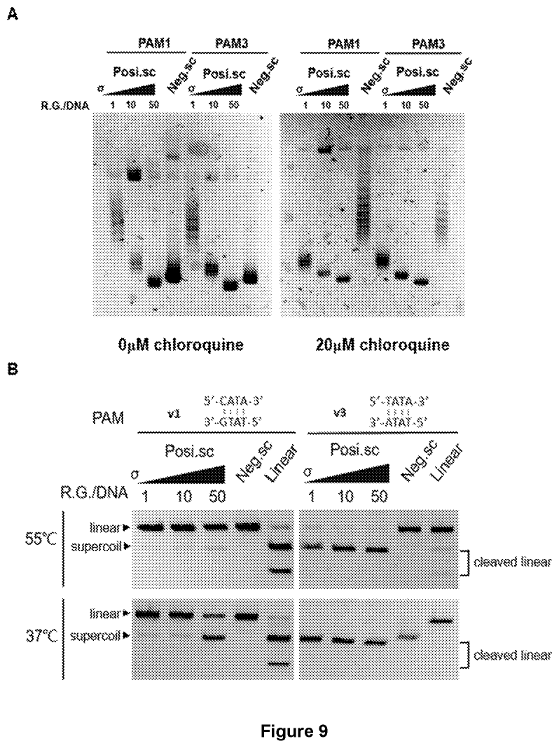

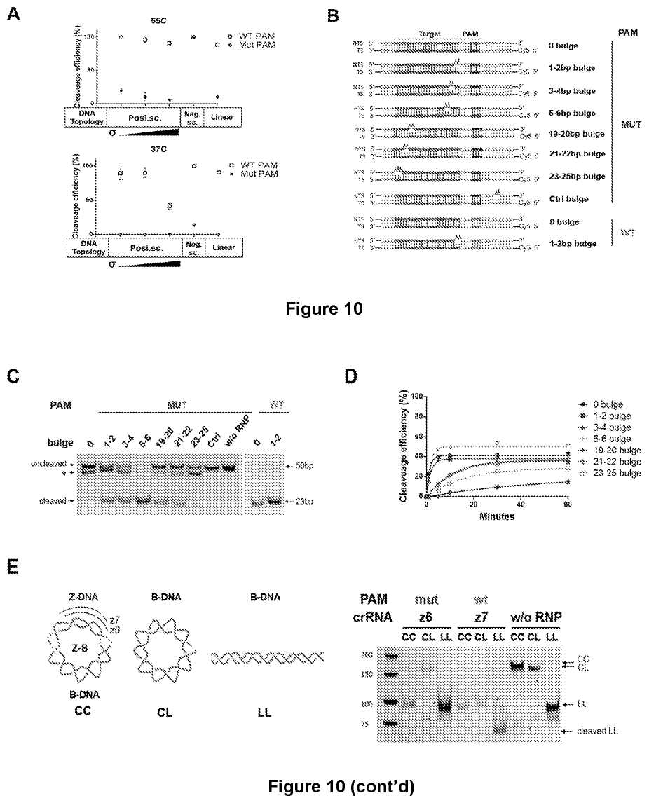

Among the many factors that regulate Cas9 activity, DNA unwinding and the subsequent R-loop formation are the primary determinant. Protein structure analysis have suggested that the hydrogen bond interaction between PAM sequences and the cas9 protein is crucial to unwind DNA helix and allow R-loop formation. Mutation in PAM sequencing completely abolished R-loop formation. DNA duplex unwinding can also be regulated by negative or positive torsional strain in the DNA. Negative supercoiling occurs when the right-handed double-helix DNA is twisted in a left-handed fashion, which preferentially underwinds the DNA helix. On the other hand, positive supercoiling involves twisting in a right-handed orientation, resulting overwinding of the helix and the generation of positive torsional strain. To determine how torsional strain regulates Cas9 activity, we first generated different levels of positive supercoiled DNA by treating negative supercoiled DNA with reverse gyrase and validated the preparation in agarose gel (FIG. 9A). Similar to relaxed linear DNA, the mutation in PAM completely abolishes AtCas9 activity against positive supercoiled substrates (FIG. 10A and FIG. 9B). These results suggest that the negative torsional strain residing in negative supercoiled isomers strongly promotes PAM-independent activity of AtCas9, whereas a positive strain in positive supercoiled DNA represses AtCas9 activity.

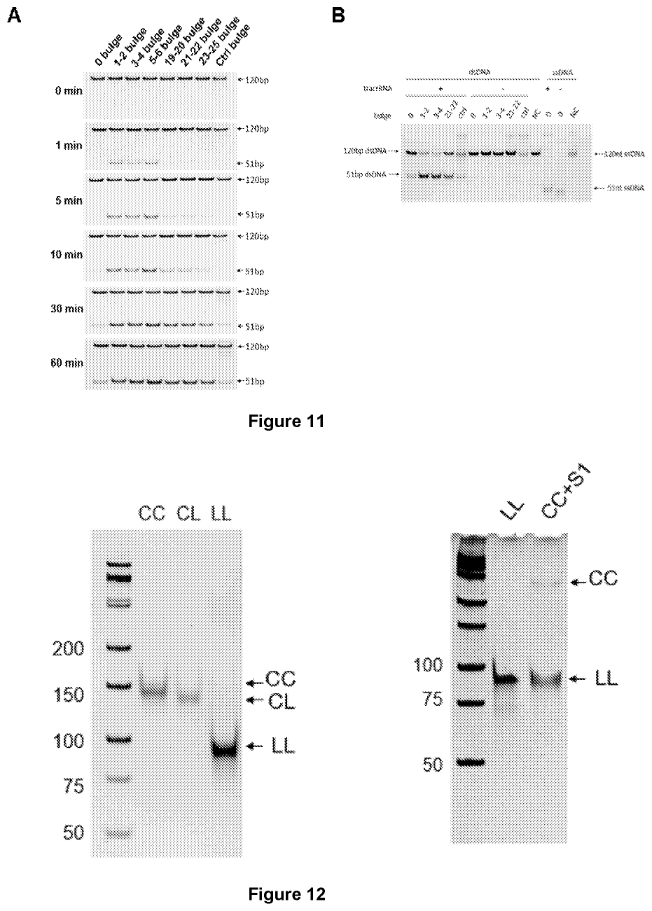

We reasoned that the negative torsional strain present in underwound DNA plays an important role in facilitating DNA unwinding. To test this hypothesis, we generated a series of bulged oligonucleotides with two-base mismatch in the non-targeting strand (NTS) spacing away from the PAM (FIG. 10B), which represent structures that mimic underwound DNAs. Consistent with our expectation, a two-base mismatch in the linear dsDNA target was sufficient to dramatically enhance PAM-independent cleavage by AtCas9 (FIG. 10C). To rule out the possibility that a 2-base bulge may have an impact on overall DNA topology in a 50-nt oligonucleotides, we synthesized a longer 120-nt oligonucleotides and performed kinetic analysis. As expected, AtCas9 cleaves bulged linear dsDNA at least 30-fold faster than no bulge control when PAM is mutated (FIG. 10D and FIG. 11A). Unlike ssDNA cleavage, this cleavage of bulged linear dsDNA also required the presence of tracrRNA (FIG. 11B).

DNA topology plays an essential role in regulating gene transcription. In nature, DNA has three topological forms including B-form, Z-form, and A-form. B-form is the most abundant and stable right-handed double helix, whereas Z-form is a less stable left-handed double helix. Z-DNA is usually composed of alternating purines and pyrimidines and tends to form in eukaryotic cells behind the transcription active site. Z-DNA is more stretched containing 12 base pairs per turn and is considered underwound topoisomer compared with B-form. We contemplated that Z-DNA could enhance PAM-independent activity. We engineered an 89-nt B-Z hybrid mini-circle DNA (FIG. 12) and performed in vitro cleavage using crRNA that matches to the Z-DNA region. Z-DNA was completely cleaved by AtCas9 whereas B-DNA was resistant to cleavage when PAM was mutated (FIG. 10E). Collectively, these data suggest that negative torsional strain in the underwound DNA structure plays a pivotal role in facilitating PAM-independent activity of AtCas9.

### Example 4. DAtCas9 has Higher Binding Affinity Towards Underwound DNA

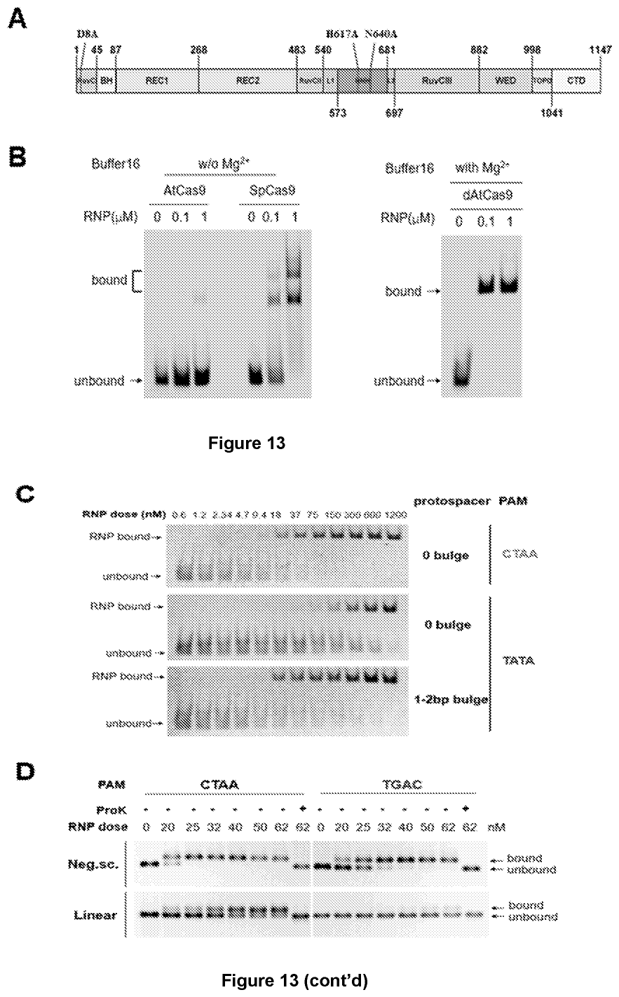

To investigate mechanisms underlying DNA structure-mediated AtCas9 activity, we tested the hypothesis that DNA topology recruits the AtCas9 RNP complex to its targeted dsDNA. We used gel mobility shift assay to measure the binding affinity of pre-complexed RNP to different topoisomers. Unlike SpCas9 or NmeCas9, AtCas9 binding with FAM-labeled oligonucleotides depends on the presence of magnesium (FIG. 13A-B). We then introduced mutations in RuvC and HNH domain (D8A H617A N640A) and generated catalytically dead AtCas9 (dAtCas9), which displays no cleavage but remains bound to the target DNA (FIG. 13A-B). Therefore, dAtCas9 was used to measure its binding affinity towards various topoisomers. PAM mutated oligonucleotide substrates substantially reduced the binding affinity of AtCas9 RNP complex, whereas introducing a two-base bulge greatly enhanced the binding affinities to levels comparable to WT PAM substrate (FIG. 13C). When PAM was mutated in the linear isomer, no binding was observed in linear isomer but only slightly decrease in binding affinity was observed with negative supercoiled DNA (FIG. 13D). Moreover, using WT PAM as the substrate, AtCas9 RNP exhibited ˜3-fold higher binding affinity towards the negative supercoiled DNA compared to linear isomer (FIG. 13D). These data suggest that the AtCas9 RNP complex possesses a robust binding capability to DNA topoisomers that shared an underwound duplex helix structure. Together, these data reflect the superior effect of DNA topology on AtCas9 activity in binding with a target PAM-MUT dsDNA which therefore enables guide RNA strand invasion and R loop formation.

### Example 5. Torque Residing within DNA Topology is Universal to the Regulation of Cas Activity

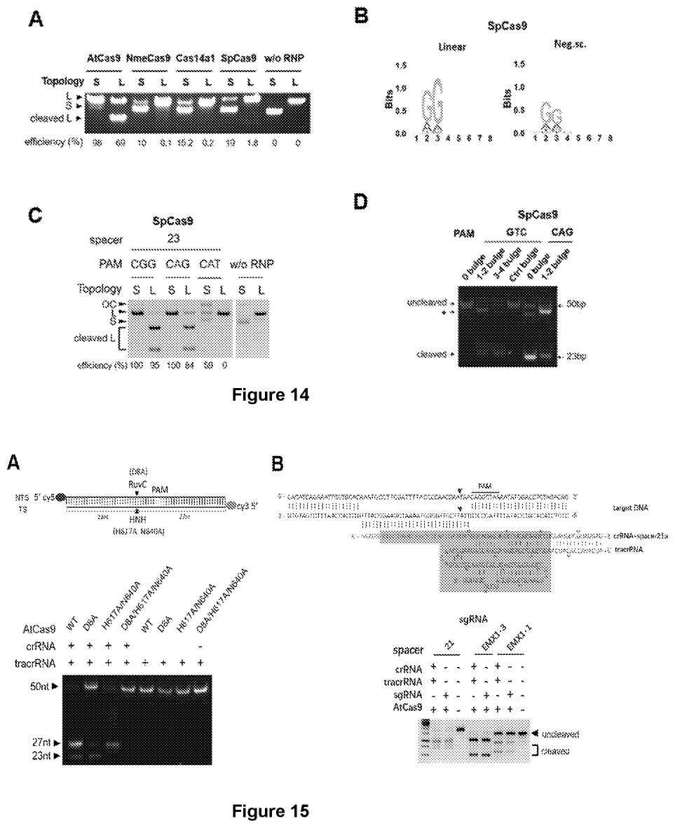

To investigate whether DNA topology is also essential for other cas orthologues, we performed in vitro PAM library cleavage assay with other CRISPR/Cas systems. SpCas9 (type II-A), NmeCas9 (type II-C) and Cas14a1 (type V) all showed ˜10-100 fold increase of cleavage activity against negative supercoiled PAM library compared with relaxed isomers (FIG. 14A). Deep sequencing of SpCas9 PAM requirement of linear and negative supercoil topoisomers showed that negative supercoiled are less restrictive than linear dsDNA for PAM preference of GG at position 2 and position 3 (FIG. 14B). Mutating PAM (CAT) for SpCas9 completely abolished its activity against linear DNA but only displayed a 40% decrease in cleavage activity against supercoiled DNA (FIG. 14C). In addition, we generated a 2 base-pair bulged DNA in PAM MUT linear DNA and tested SpCas9 activity. Similar to AtCas9, ˜50% bulged DNA were cleaved by SpCas9 even when PAM is mutated (GTC) whereas the no bulge control showed 0% cleavage (FIG. 14D). Together, these data demonstrate the importance of DNA topology in regulating SpCas9 activity when PAM is mutated.

### Example 6. Genome-Editing Activity of AtCas9 in Mammalian Cells

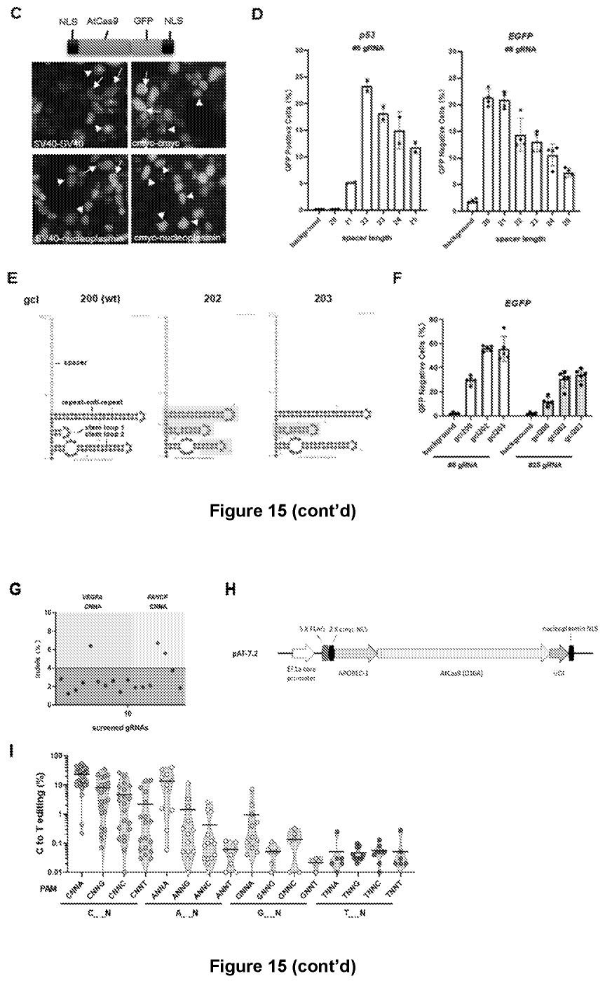

Similar to other Cas9 orthologues, the two nuclease domains of AtCas9, HNH and RuvC are responsible to cleave the target strand and non-target strand at 3 bp upstream of the PAM, respectively (FIG. 15A and FIG. 5E). To determine whether the thermophilic AtCas9 is active in mammalian cells, we first identified the minimal tracrRNA and crRNA requirement and engineered the dual RNA system into single-guide RNA system (FIG. 15B). To determine whether the thermophilic AtCas9 is active in mammalian cells, we codon-optimized the AtCas9 and compared different nuclear localization signals (NLSs) to ensure nuclear delivery in mammalian cells (FIG. 15C). A dual NLS (cmyc-nucleoplasmin) was selected and sgRNA driven by U6 promoter was tested for in vivo cleavage in two different 293T reporter cell lines. One has a single copy EGFP and the other reporter has a truncated p53 sequence followed by 1 bp frameshift EGFP. sgRNAs targeting EGFP or p53 were designed and transfected into corresponding cell lines. EGFP disruption or activation was analyzed by FACS as a readout for editing efficiency. First, we optimized the spacer length by transfecting various length of spacer targeting p53 or EGFP locus (FIG. 15D). When the spacer length is 21-nt or 22-nt, the editing efficiency of AtCas9 is optimal, resulting ˜25% EGFP disruption or activation in two reporter cell lines (FIG. 15D). Second, we engineered the sgRNA scaffold by extending the stem loop1 with a bulge structure according to NmeCas9 sgRNA structure or truncating the repeat-anti-repeat and stem loop2 region (FIG. 15E). By designing two sgRNAs targeting EGFP locus, we found extending stem loop1 with an additional bulge structure (203 construct) led to 1.8 fold increase in EGFP disruption compared with wildtype (200 construct). Truncating the repeat-anti-repeat and stem loop2 region (202 construct) did not further enhance editing (FIGS. 15E and 15F). Till now, we have optimized AtCas9 and its gRNA to be able to achieve 60% efficiency at a lentiviral-generated EGFP locus. To test editing efficiency in other endogenous locus, we screened 7-10 sgRNAs targeting FANCF or VEGFA locus and performed TIDE analysis. Of 17 gRNAs tested, only 3 showed limited editing <7% (FIG. 15G) and the remaining 14 gRNAs have very low or no efficiency beyond the detection limit of TIDE assay. Collectively, these results suggest that AtCas9 is able to edit mammalian genome but with low efficiency at endogenous locus. The low efficiency of AtCas9 may result from its compromised activity at 37° C., or the presence of chromosome structure affects its accessibility to the target site.

One of the striking characteristics of AtCas9 is its relaxed PAM of CNNN and RNNA (R=A, G) and its near PAMless cleavage against underwound DNA at its optimal condition. Considering high constraints of PAM selection for base editors, we explored the application of AtCas9 in base editing. We first constructed the cytosine base editor (CBE) expression vector of AtCas9 (named pAT7.2, FIG. 15H), and screened near 280 gRNAs from four different loci (VEGFA, RUNXI, C-MYC and EGFP) in 293T cells covering 16 PAM combinations (differing in position 5 and position 8) (FIG. 15I). When presented with CNNA PAMs, 31 out of 33 gRNAs showed effective C-to-T editing, up to 55% efficiency (mean value of 23%); when the PAM is CNNG or ANNA, the mean efficiency was ˜10%; when PAM is CNNY (Y=C or T), ANNG and GNNA, the mean editing efficiency dropped down to ˜2% (FIG. 5G); no editing is observed when PAM is TNNN, RNNT (R=A or G). In sum, AtCas9-base editor is able to mediate effective C-to-T editing across multiple loci with broad PAM preference of CNNN and RNNA (R=A, G).

### Example 7. Engineering of AtCas9 PI Domain Capable of Targeting Broader PAM Variants

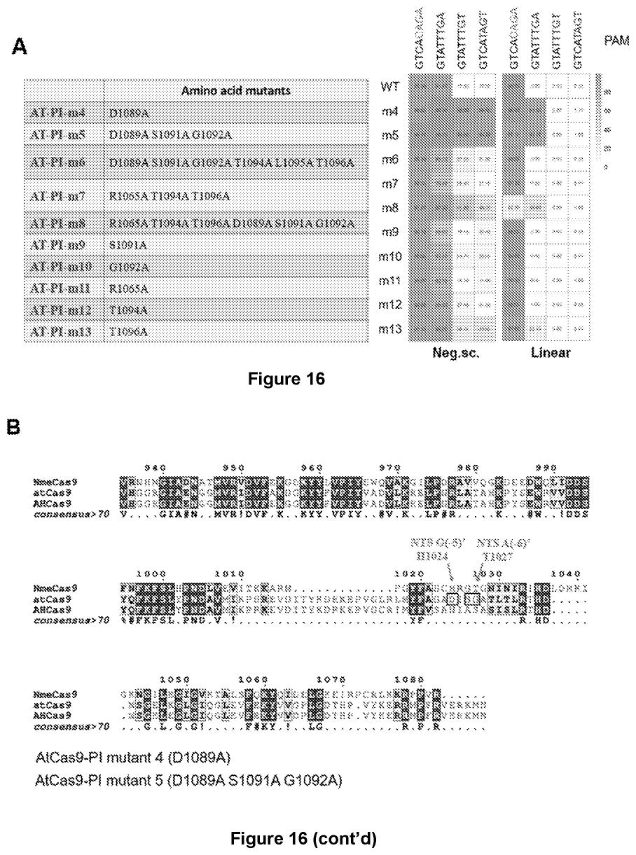

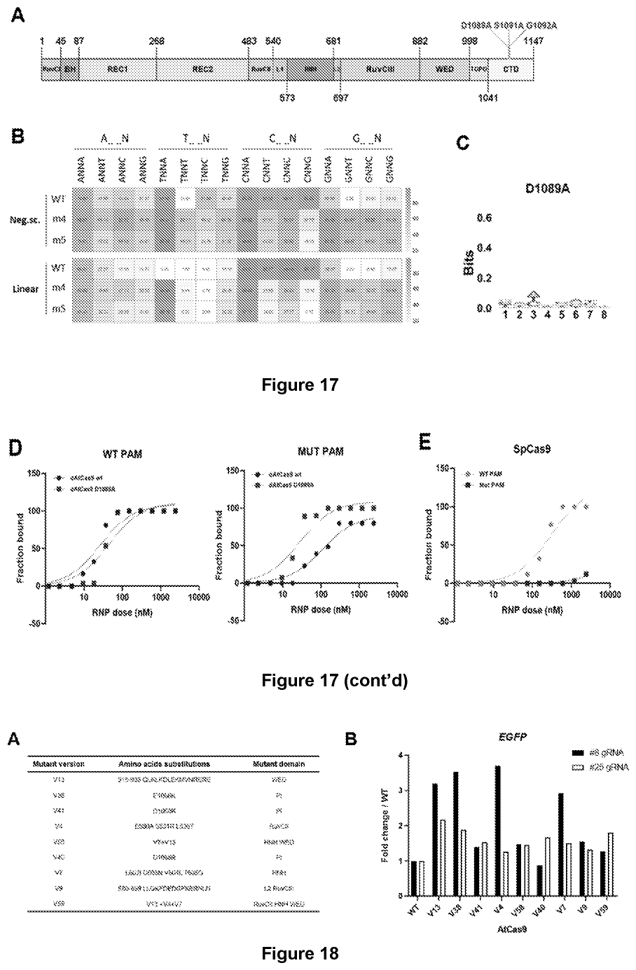

Structural analysis of SpCas9 and NmeCas9 suggested that the interaction between PAM-interacting (PI) domain of Cas9 and the PAM base is important to initiate R-loop formation. To determine whether this model can be applied to AtCas9, we determined whether mutation of the PI domain of AtCas9 affects its cleavage activity. AtCas9 and AhCas9 share 98% protein sequence identity with main difference in the PI domain. Therefore, we focused on the 7 amino acids within the PI domain, verified the function of these AtCas9 protein mutants on different PAM substrates by in vitro cleavage assays (FIG. 16A), and further filtered them against structurally validated PAM interacting amino acids of NmeCas9 (FIG. 16B). Two AtCas9 protein mutants, PI-m4 (D1089A, named as PI-m4) and PI-m5 (D1089A S1091A G1092A, named as PI-m5), were generated and tested at 55° C. (FIGS. 17A and 17B). In addition to maintaining higher cleavage activity on negative supercoiled substrates than linear dsDNA, both PI-V4 and PI-V5 enhanced the cleavage efficiency of DNNN (D represents A, G or T) PAM substrates and reduced the cleavage efficiency of CNNN PAM substrates (FIG. 17B). Furthermore, in vitro PAM identification assay also proved that PI-m4 (D1089A) mutant has no PAM preference (FIG. 17C). Through gel mobility shift assays, we found that D1089A AtCas9 showed the same binding ability as wild-type AtCas9 for wt PAM dsDNA, but D1089A AtCas9 showed ˜4-fold higher binding affinity towards mutant PAM dsDNA compared to wild-type AtCas9 (FIG. 17D). By contrast, SpCas9 RNPs hardly bind to PAM mutant substrates (FIG. 17E). These findings suggest that PI domain mutant 4 (D1089A) can enhance the binding affinity of AtCas9 to PAM mutant dsDNA and thus improve its cleavage efficiency.

### Example 8. Engineering of AtCas9 Protein to Improve Activity and Target Broader PAM Variants in Mammalian Cells

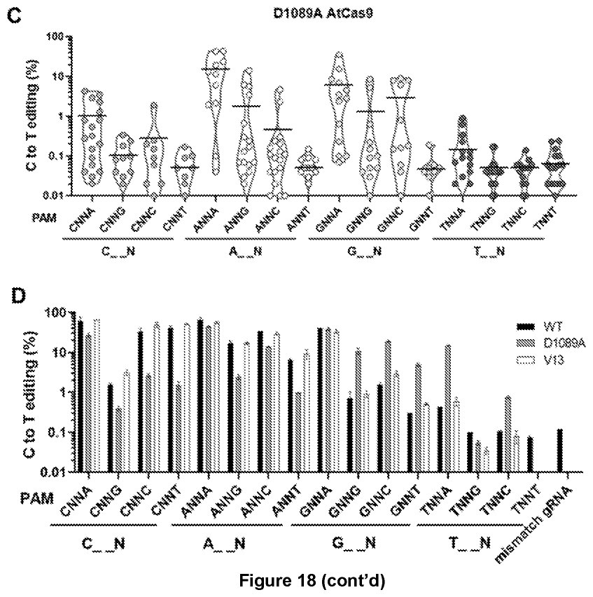

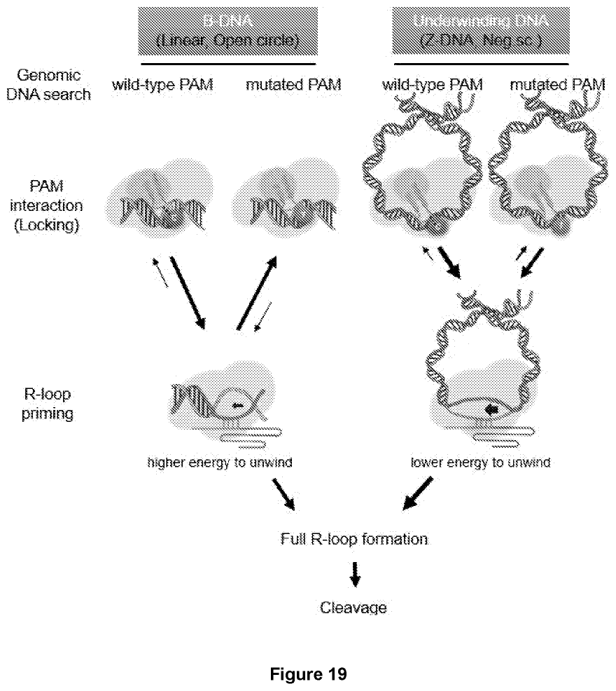

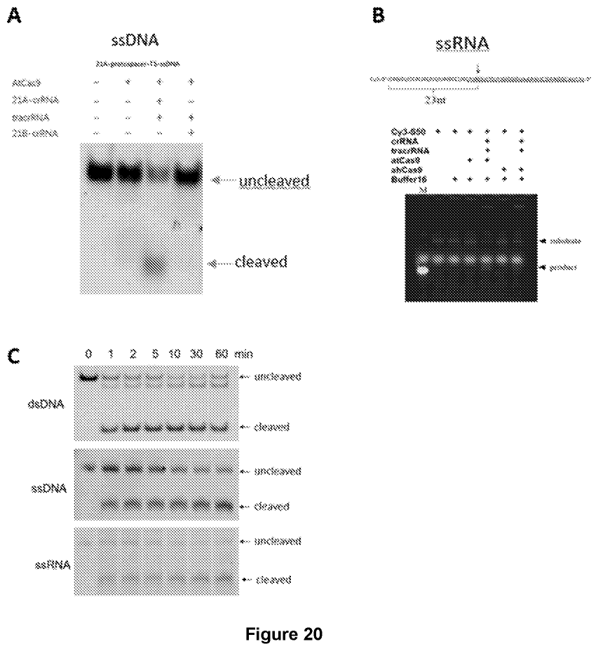

In vitro cleavage experiments confirmed that AtCas9 had the optimal activity at 55° C., while the cutting activity decreased at 37° C. (FIG. 1C). In order to improve the cleavage activity of AtCas9 at 37° C., we analyzed the structural of the NmeCas9-sgRNA complex and NmeCas9-sgRNA-dsDNA complex, summarized a series of regions with conformational rearrangement, and replace the amino acids corresponding to AtCas9 with the sequence of NmeCas9. sgRNAs targeting EGFP were designed and transfected into a single copy EGFP cell line, and analyze the changes in genotypes after editing cells with different mutants by TIDE. Through preliminary screening, we selected several mutant proteins that can increase cleavage activity in mammalian cells (FIGS. 18A and 18B). Among them, when a small part of the amino acid sequence in the WED domain is replaced (V13), its cleavage activity of AtCas9 is increased by 2-3 times (FIG. 18B), indicating that this sequence is more important for the activity of AtCas9 at 37° C.

Next, we explored the application of D1089A AtCas9 in base editing, and screened near 280 gRNAs from four different loci (VEGFA, RUNXI, C-MYC and EGFP) in 293T cells covering 16 PAM combinations (differing in position 5 and position 8) (FIG. 18C). Compared with wild-type AtCas9 (FIG. 15I), the D1089A mutant has lower editing efficiency for CNNN PAM, but improves the recognition of GNNN and TNNA PAM, and has almost no effect on ANNN and TNNB PAM (B=G, C and T). When co-transfecting different variants of AtCas9 expressing plasmid and negative supercoiled substrates vary in PAM combinations, we found AtCas9-CBE is able to generate editing in 13 PAM combinations with variable efficiencies (FIG. 18D). The C-to-T base editing efficiency of the V13 mutant is basically the same as wild-type AtCas9 (FIG. 18D), suggest that the V13 mutant may affect the allosteric of the nuclease domain, which enhances the cleavage activity, but does not affect its recognition and binding to target dsDNA. For the D1089A mutant, base editing efficiency for GNNN and TNNM PAM has been increased, but for other PAMs, especially CNNN PAM, the editing efficiency is reduced (FIG. 18D). We speculate that the binding of D1089 to PAM sequence may have both hydrogen bond interaction and repulsion, prompting the identification of different sequences PAM. Further exploration of the structure of AtCas9 is needed in the later stage.

Base editors are one of the most powerful tools to correct genetic mutations, but the PAM restriction has become a rate limiting step to select effective gRNAs. In this Example, we identified a thermostable endonuclease AtCas9, originally found in a thermophile with optimal temperature at 55° C. To our knowledge, it has the most relaxed PAM of CNNN and RNNA (R=A, G) at its optimal temperature, covering 68% of sequences. When presented with negative supercoiled substrates, the overall cleavage showed 3-fold increase compared with linear dsDNA and the PAM preference extended to MNNN, TNNM and GNNA (M=C, A, V=non T), covering 94% of sequences. Moreover, AtCas9 is able to cleave all PAM combinations in E. coli when substrates are negative supercoil topology. It is quite striking that AtCas9 is active in mammalian cells. AtCas-base editor showed high editing efficiency in mammalian cells and covered a wide range of PAM sequences (CNNN and RNNA, R=A, G).

Our finding suggested that PAM is not a yes/no gate that distinguishes self or non-self. Instead, PAM is a continuous sequence combination, varying in the binding strength with Cas9. As the binding strength of AtCas9 to PAM increases, such as in the case of strong PAM, the degree of DNA unwinding proceeds to fully complementary pairing with spacer, thereby triggering effective cleavage. As the binding strength decreases, for example in the cases of intermediate or weak PAM, the degree of DNA unwinding decreases, and little or no cleavage is observed. The other factor, DNA torque which functions to inhibit DNA unwinding, also plays a critical role in regulating AtCas9 activity. Naturally existing underwound DNA such as negative supercoil or Z-form dsDNA have smaller torque, and are easier to unwind when compared with B-form dsDNA. When torque is greater than the PAM binding strength, DNA unwinding can be inhibited, such as in the case of linear dsDNA with weak PAM. When torque is smaller, such as underwound DNA, weak PAM dsDNA could trigger DNA unwinding and lead to cleavage. Nevertheless, when the strength of PAM binding is strong enough, the effect of torque on DNA unwinding can be overcome.

When editing mammalian genome, Cas9 activities vary between locus. Studies have indicated that chromosomal structure, histones and epigenetic marks may influence the accessibility for Cas9. Our study has added another important factor-DNA topology, in regulating Cas9 activity. For example, if gRNAs are designed at regions that tend to form positive supercoil, the activity is likely to be compromised. If gRNAs are designed towards underwound regions, higher editing is likely to be achieved. AtCas9 has shown high sensitivity against DNA topology particular when presented with weak PAM (TNNA). The ability to program AtCas9 to respond to various levels of torsional strain in DNA may further provide new opportunities for learning the dynamics of DNA structure.

### Example 9. Materials and Methods

### RNA In Vitro Transcription

RNA was in vitro transcribed using synthetic DNA oligos carrying a T7 promoter sequence. After transcription with T7 RNA polymerase at 37° C. for 1 hour, tracrRNA, crRNA or sgRNA were purified using column or gel purification kit (NEB or ZYMO) according to the manufactory protocols. Primers and oligonucleotides used in this study were listed in Table 1.

### Protein Purification

6×His tagged AtCas9 gene was synthesized and cloned into pACYCDuet-1 vector (GenScript). The recombinant plasmid was transformed into E. coli BL21 (DE3) and protein expression was induced by adding 0.5 mM IPTG. After incubating at 18° C. for 16h, cell pellets were resuspended in lysis buffer (20 mM Tris-HCl, 500 mM NaCl, 10% glycerol, pH7.4) and lysed by sonication (Scientz). Supernatant was collected after centrifugation and then filtered with 0.22 micron filters. Affinity purification followed by size exclusion chromatographic step were performed for protein purification. In brief, clarified lysate was loaded to HisTrap HP (GE Healthcare) in NGC Quest 10 Chromatography System (Biorad). Column is pre-balanced in lysis buffer. Protein was eluted in buffer B1 (20 mM Tris-HCl, 500 mM NaCl, 500 mM Imidazole, 10% glycerol, pH7.4) using gradient program. Different elution fractions were collected and then verified by SDS-PAGE to identify target protein. Affinity purified protein was then loaded to Superdex 200 Increase 10/300 GL column (GE Healthcare) in buffer B2 (20 mM Tris-HCl, 200 mM NaCl, 20% glycerol, pH7.4). Eluted protein was concentrated by centrifugal filters (Millipore) and stored in buffer B2 at −80° C. AtCas9 D8A, H617A/N640A, D8A/H617A/N640A and PI-m5, PI-m8 mutants were generated using the site-directed mutation PCR and confirmed by DNA sequencing. AhCas9 gene were synthesized and cloned into the same expression vector (GenScript). The proteins were purified following the same procedure as for the wild-type AtCas9 protein.

T7 RNA polymerase, SpCas9, NmeCas9 and Cas14a1 were purified according to the previous studies. In short, T7 RNApolymerase gene and SpCas9/NmeCas9/Cas14a1 gene were synthesized (GenScript) and cloned into pET30c or pET28a vector respectively. T7 RNA polymerase was induced at 37° C. for 4h after adding 1 mM IPTG, whereas SpCas9/NmeCas9/Cas14a1 was induced at 25° C. for 10h after adding 0.5 mM IPTG. Reverse gyrase gene were synthesized and cloned into pET28a vector (GenScript), and the protein was induced at 20° C. for 14 h under 0.1 mM IPTG. The protein purification steps were the same as AtCas9.

### In Vitro Cleavage Assay

Unless noted elsewhere, purified AtCas9 protein (50 nM) and crRNA:tracrRNA duplex (50 nM, 1:1) were mixed in 1×buffer16 (10 mM KCl, 20 mM HEPES, 10 mM MgCl₂, 0.5 mM DTT, 0.1 mM EDTA, pH7.9), and incubated at room temperature for 10 minutes. DNA substrate was added to the reaction system in the final concentration of 2 nM (plasmid DNA or linear dsDNA longer than 2 kb) or 10 nM (fluorescently labeled oligonucleotides) and incubated at 55° C. for 30 min. The reactions were stopped by adding 1 μl Proteinase K (Thermo Fisher) at 55° C. for 10 min. Cleaved products were resolved by 0.8% TAE agarose gel and visualized by ethidium bromide staining for plasmid DNA, 12% native PAGE or denature PAGE for fluorescently labeled oligonucleotides. Quantification was performed using Image Lab software (Bio-rad) and cleavage efficiency were plotted using Prism 6 (GraphPad).

Each Cas orthologs (SpCas9, NmeCas9 or Cas14a1) in complex with its cognate dual-RNA to cleave supercoil and linear substrates under their optimal cleavage conditions. SpCas9 and NmeCas9 cleaved dsDNA in buffer16 (10 mM KCl, 20 mM HEPES, 10 mM MgCl₂, 0.5 mM DTT, 0.1 mM EDTA, pH7.9) and buffer6 (50 mM KCl, 20 mM HEPES, 10 mM MgCl₂, 1 mM DTT, pH7.5) at 37° C., respectively. Whereas Cas14a1 reacted at buffer (25 mM NaCl, 20 mM HEPES, 5 mM MgCl₂, 1 mM DTT, pH7.5) and 46° C.

### In Vivo PAM Screen

The PAM Plasmid library containing the protospacer 21a and 8 nucleotides randomized PAM sequences were synthesized and cloned into pUC19 vector (GenScript). The pooled plasmid library (100 ng) were transformed into electrocompetent E. coli harbouring a AtCas9 locus (pACYC184-AtCas9), SpCas9 locus (pCas9-21) or a control plasmid with no locus (pACYC184). After transformation, cells were grown in LB medium (10 g/L tryptone, 5 g/L yeast extract, 10 g/L NaCl, pH 8) supplemented with 0 mM, 0.1 mM, 1 mM, 5 mM, or 10 mM MgCl₂ for 16 h at 37° C. Selection antibiotics ampicillin (50 g/ml) and chloramphenicol (25 g/ml) were used. Plasmid DNA was extracted and purified using plasmid mini kit (Omega). The target PAM region was amplified with primers containing adapters for Illumina NovaSeq. NovaSeq reads were filtered by an average Phred quality (Q score) >25. The 8 nucleotides randomized PAM was extracted and analyzed with Python script. Normalization was performed against control sample to calculate sequencing or PCR bias. The figures were draw by R.

### In Vitro PAM Screen

The PAM plasmid library described previously were treated with BsaI or Nt.BspQI (NEB) to generate linear or open circle topology respectively. Column purified linear, open circle or negative supercoiled PAM library DNA were then digested in buffer 16 (10 mM KCl, 20 mM HEPES, 10 mM MgCl₂, 0.5 mM DTT, 0.1 mM EDTA, pH7.9) with AtCas9-crRNA-tracrRNA complex at 50 nM concentration and incubated at 55° C. for 2h. Cleaved products were resolved on 1% TAE agarose gel and cleaved bands were gel extracted. The target PAM region was amplified with adapters for NovaSeq. NovaSeq reads were required to be filtered by an average Phred quality (Q score) at least 25. The 8 nucleotides randomized PAM was extracted and analyzed with Python script. Raw reads were normalized according to the control which is digested by restriction enzyme EcoRI. The seqlogo figures were draw by R.

### Preparation of Positive Supercoiled Plasmid and Z-DNA

Positive supercoiling: 5 nM negative supercoiled plasmid pCE2-PAM1 or pCE2-PAM3 was incubated with various concentrations of Reverse Gyrase (5 nM, 50 nM, 250 nM) in 1×RG buffer (35 mM Tris-HCl, 0.1 mM Na₂EDTA, 30 mM MgCl₂, 2 mM DTT, 1 mM ATP) in a final volume of 201. After incubating at 80° C. for 10 minutes, the samples were purified and analyzed on 1% TAE agarose gel with 0 μM or 20 μM chloroquine (Sigma). Gels were stained with ethidium bromide.

Z-DNA: The preparation of Z-DNA was modified from previous studies. Two single-stranded DNA circles were prepared separately and then hybridized to form a Z-form and B-form DNA hybrid⁷. In brief, 89 nt single-stranded DNA with 5′-phosphate and 3′-hydroxyl groups were synthesized and circularized using CirLigase (Lucigen) to form DNA circle. The circularization reaction was carried out at 60° C. for 2h, and then followed by ExonucleaseI(NEB) to remove the linear DNA at 37° C. for 2 h. The cyclization products were recovered and the single-stranded DNA circle (cF-89, circular form of 1-F89) and its complementary circle (cR-89, circular form of 1-R89) were annealed in 1×annealing buffer (10 mM HEPES, 10 mM MgCl₂, pH7.5) to generate a B-form and Z-form hybrid double-stranded DNA circle (CC). Samples were analyzed on 8% native PAGE, and hybrid double-stranded DNA circles were column purified using Zymo DNA Clean & Concentrator kit (Zymo). Validation of Z-DNA were performed by treating with S1 nuclease (Thermo Fisher), which recognized Z-B junction and resulted a double-stranded break at the recognition site⁷. 0.2 μM Z-B chimera hybrid was treated with S1 nuclease (2U) in 20 μl 1×reaction buffer. After incubating at room temperature for 1h, the reaction was terminated by adding 2 μl EDTA (0.5M, pH8.0) and heat-inactivated at 70° C. for 10 min. The linear products were resolved by 12% native PAGE.

### Electrophoretic Mobility Shift Assay (EMSA)

dAtCas9 and sgRNA were mixed at 1:2 molar ratio in 1×buffer16. After incubation at room temperature for 10 minutes, the RNP mixture were diluted to various concentration ranging from 1. μM to 0.6 nM and 4 nM cy5-labeled oligonucleotides was added. Binding reactions were carried out at 55° C. for 30 minutes. Samples were resolved on 4% native TBE-PAGE for oligonucleotides. For plasmid substrate binding assay, we first generated 1551 bp mini plasmid from pCE2-PAM1 or pCE2-PAM3 vector by digesting with RSRII and PciI enzymes. Mini plasmids were sequence verified and labeled with Cy5 kit according to the manufactory protocol (Mirus Bio). Binding reactions were resolved on 0.8% agarose gel in 1×sodium boric acid (SB) buffer (8.6 mM sodium borate, 45 mM boric acid, pH8.3) for 11 h at 20 mA at 4° C. Gels were imaged by Biorad Chemidoc MP imager (Bio-rad).

PAM-Independent Cleavage in E. coli

AtCas9 CRISPR locus containing 21a protospacer sequences and chloramphenicol expression fragment was amplified from pACYC184-AtCas9 using the primer pairs yz101-LHA-lacA-F and yz102-LacA-LHA-R, and this fragment was integrated into the LacA locus in E. coli MG1655 genome. The newly generated E. coli strain, MG1655-AtCas9, was transformed with the various PAM mutant plasmids which contain a kanamycin resistance marker. The transformed cells were plated on chloramphenicol (25 g/mL) and chloramphenicol (25 g/mL)/kanamycin (50 g/mL) LB plates to calculate total CFU and resistant CFU respectively. Transformation frequencies were determined as antibiotic resistant cfu/ml and total cfu/ml from six independent experiments.

### Cell Culture, Transfection and GFP Detection

Human HEK 293T cells and its derivative reporter cell line were maintained in DMEM with 10% FBS and 1% Penicillin/Streptomycin in a 37° C. incubator with 5% CO₂. HEK293T-EGFP cell line was generated by stably incorporating a EF1α-EGFP vector into genome by lentivirus. HEK293T-p53-(+1frame shifted)-EGFP cell line was generated by stably incorporating a EF1α-p53-(+1frame shifted)-EGFP vector by lentivirus. Single copy integration colonies were selected by FACS sorting (BD FACSAria III) and cells were maintained on hygromycin supplemented selection medium.

For in vivo genome editing experiments, 2 ug of AtCas9 and 1 ug of sgRNA expression plasmids, pAT301 and gcl203, were mixed in 20 ul electroporation buffer and transfected into 4×10⁵ cells using Lonza 4D-Nucleofector following the manufacturer's protocol (Lonza), and then the electroporated cells are seeded into a 24-well plate. FACS analysis of GFP positive cells for p53 locus or GFP negative cells for EGFP locus were performed 5 days post transfection using Novocyte (Agilent). Genomic DNA was extracted using Hipure Tissue DNA Mini Kit (Magen) for endogenous loci VEGFA and FANCF, and target region was PCR amplified and sent for amplicon sequencing (Illumina).

For base editor experiments, 1×10⁵ cells were seeded into 24-well plates one day prior to transfection. 600 ng of AtCas9-CBE or AtCas9 variants expression plasmids and 400 ng of sgRNA expression plasmids (gcl203) were co-transfected into ITEK293T cells by calcium phosphate precipitation method. When editing negative supercoiled substrates, 600 ng pAT7.2 and 400 ng gcl203 harboring spacer 36 were co-transfected with 1 ng plasmids encoding PAM variants into HEK293T cells. Genomic DNA or plasmids was extracted four days post transfection using 50 mM NaOH at 95° C. for 10 minutes, and neutralized with 1M Tris-HCl (pH8.0). The editing locus were amplified and prepared for amplicon sequencing (Illumina).

Imaging of AtCas9 nuclear localization were performed by fixing transfected cells with 200 formaldehyde for 10 min at room temperature. Cells were counterstained with Hoechst (Life Technologies) and imaged under fluorescence microscopy (Nikon).

TABLE 1
RNA and DNA used in this study.

| Name | Sequence (5′ to 3′)§ | Used in |
|---|---|---|
| tracrRNA- | AUCAUACCACAUCAAGGCUUGCGAGGUUGCUAUGAUAAGGCAACAGGCCGCAA | FIG. 3, 5, |
| At/Ah | AGCACUGACCCGCAUUCCAAUGAAUGCGGGUCAUCUACUUUUU (SEQ ID | 11A, 11C-D, |
| | NO: 1) | 15D, 17A-C, |
| | | 1C, 2, 4A, 4C, |
| | | 6, 7, 8, 9, 10B, |
| | | 12, 18 |
| 21a crRNA- | AAAUGCCUUCGAUUUUACCCCAACGAAUAAGUCAUAGUUCCCUCACAAGCCUC | FIG. 3, 5, |
| At/Ah | GAUGUGGUAUGAU (SEQ ID NO: 2) | 11A, 11C-D, |
| | | 15D, 17A-C, |
| | | 1C, 2, 4A, 4C, |
| | | 6A, 7A, 7C, 8, |
| | | 9, 10, 12, 17, |
| | | 18 |
| tracrRNA- | AAAUGAGAACCGUUGCUACAAUAAGGCCGUCUGAAAAGAUGUGCCGCAACGCU | FIG. 15E |
| Nme | CUGCCCCUUAAAGCUUCTGCUUUAAGGGGCUUUUU (SEQ ID NO: 3) | |
| 21a crRNA- | CUUCGAUUUUACCCCAACGAAUAAGUUGUAGCUCCCUUUCUCAUUUCG (SEQ | FIG. 15E |
| Nme | ID NO: 4) | |
| tracrRNA- | UUCACUGAUAAAGUGGAGAACCGCUUCACCAAAAGCUGUCCCUUAGGGGAUUA | FIG. 15E |
| Cas14a1 | GAACUUGAGUGAAGGUGGGCUGCUUGCAUCAGCCUAAUGUCGAGAAGUGCUUU | |
| | CUUCGGAAAGUAACCCUCGAAACAAAUUCAUUUUUCCUCUCCAAUUCUGCA | |
| | (SEQ ID NO: 5) | |
| 21a crRNA- | UGCAGAACCCGAAUAGACGAAUGAAGGAAUGCAACAAAUGCCUUCGAUUUUAC | FIG. 15E |
| Cas14a1 | CCCAACGAAUAA (SEQ ID NO: 6) | |
| 21a | GAUUUUACCCCAACGAAUAAGUUUUAGAGCUAGAAAUAGCAAGUUAAAAUAAG | FIG. 3A, 15G, |
| sgRNA- | GCUAGUCCGUUAUCAACUUGAAAAAGUGGCACCGAGUCGGUGCUUUUUU | 2, 14A |
| Sp | (SEQ ID NO: 7) | |
| 23 sgRNA- | UUACCCCAACGAAUAAGUACGUUUUAGAGCUAGAAAUAGCAAGUUAAAAUAAG | FIG. 15F, |
| Sp | GCUAGUCCGUUAUCAACUUGAAAAAGUGGCACCGAGUCGGUGCUUUUUU | |
| | (SEQ ID NO: 8) | |
| 21a | AAAUGCCUUCGAUUUUACCCCAACGAAUAAGUCAUAGUUCCCUCACAAGCCUC | FIG. 15A, |
| sgRN- | GAUGUGGUAUGAUGAAAAUCAUACCACAUCAAGGCUUGCGAGGUUGCUAUGAU | 14A, B |
| At | AAGGCAACAGGCCGCAAAGCACUGACCCGCAUUCCAAUGAAUGCGGGUCAUCU | |
| Full length | ACUUUUU (SEQ ID NO: 9) | |
| 21 | AAAUGCCUUCGAUUUUACCCCAACGAAUAAGUCAUAGUUCCCUCACAAGCCUG | FIG. 17B |
| sgRNA- | AAAAGGCUUGCGAGGUUGCUAUGAUAAGGCAACAGGCCGCAAAGCACUGACCC | |
| At | GCAUUCCAAUGAAUGCGGGUCAUCUACUUUUU (SEQ ID NO: 10) | |
| truncation | | |
| z6 sgRNA- | GAUCGCACAUACGUACACAUUGACCGUCAUAGUUCCCUCACAAGCCUCGAUGU | FIG. 11E |
| At | GGUAUGAUGAAAAUCAUACCACAUCAAGGCUUGCGAGGUUGCUAUGAUAAGGC | |
| | AACAGGCCGCAAAGCACUGACCCGCAUUCCAAUGAAUGCGGGUCAUCUACUUU | |
| | UU (SEQ ID NO: 11) | |
| z7 sgRNA- | ACAUACGUACACAUUGACCUAUUUAGUCAUAGUUCCCUCACAAGCCUCGAUGU | FIG. 11E |
| At | GGUAUGAUGAAAAUCAUACCACAUCAAGGCUUGCGAGGUUGCUAUGAUAAGGC | |
| | AACAGGCCGCAAAGCACUGACCCGCAUUCCAAUGAAUGCGGGUCAUCUACUUU | |
| | UU (SEQ ID NO: 12) | |
| 27m | GAAGAUCCUUUGAUCUUUUCUACGGGUCAUAGUUCCCUCACAAGCCUCGAUGU | FIG. 5B, 15B, |
| sgRNA- | GGUAUGAUGAAAAUCAUACCACAUCAAGGCUUGCGAGGUUGCUAUGAUAAGGC | 7B |
| At | AACAGGCCGCAAAGCACUGACCCGCAUUCCAAUGAAUGCGGGUCAUCUACUUU | |
| | UU (SEQ ID NO: 13) | |
| gRNA8 | GCCAGGGCACGGGCAGCUUGCCGGUGUCAUAGUUCCCUCACAAGCCUCGAUGU | FIG. 17D, E |
| | GGUAUGAUGAAAAUCAUACCACAUCAAGGCUUGCGAGGUUGCUAUGAUAAGGC | |
| | AACAGGCCGCAAAGCACUGACCCGCAUUCCAAUGAAUGCGGGUCAUCUACUUU | |
| | UU (SEQ ID NO: 14) | |
| gRNA12 | AGGGCGAGGAGCUGUUCACCGGGGUGUCAUAGUUCCCUCACAAGCCUCGAUGU | FIG. 17D, E |
| | GGUAUGAUGAAAAUCAUACCACAUCAAGGCUUGCGAGGUUGCUAUGAUAAGGC | |
| | AACAGGCCGCAAAGCACUGACCCGCAUUCCAAUGAAUGCGGGUCAUCUACUUU | |
| | UU (SEQ ID NO: 15) | |
| 6m crRNA- | CAUGAGACAAUAACCCUGAUAAAUGCUUCAGUCAUAGUUCCCUCACAAGCCUC | FIG. 6B |
| At/Ah | GAUGUGGUAUGAU (SEQ ID NO: 16) | |
| 16m | UGAAUGUCAGCUACUGGGCUAUCUGGACAAGUCAUAGUUCCCUCACAAGCCUC | FIG. 6B |
| crRNA- | GAUGUGGUAUGAU (SEQ ID NO: 17) | |
| At/Ah | | |
| 18m | ACGGCUCUCUCUUUUAUAGGUGUAAACCUUGUCAUAGUUCCCUCACAAGCCUC | FIG. 6B |
| crRNA- | GAUGUGGUAUGAU (SEQ ID NO: 18) | |
| At/Ah | | |
| 24m | ACCUGUAGACAGCAUGAAGUUAGGAUUGUGGUCAUAGUUCCCUCACAAGCCUC | FIG. 6B |
| crRNA- | GAUGUGGUAUGAU (SEQ ID NO: 19) | |
| At/Ah | | |
| 28m | CGCCAUUAACCUGAUGUUCUGGGGAAUAUAGUCAUAGUUCCCUCACAAGCCUC | FIG. 6B |
| crRNA- | GAUGUGGUAUGAU (SEQ ID NO: 20) | |
| At/Ah | | |
| 21b | UUAUUCGUUGGGGUAAAAUCGAAGGCAUUUGUCAUAGUUCCCUCACAAGCCUC | FIG. 3E, 4A, |
| crRNA- | GAUGUGGUAUGAU (SEQ ID NO: 21) | C |
| At/Ah | | |
| EMX1 | GAUGUUGAGCUCCGUUUGGGGGUAUGGUGAGUCAUAGUUCCCUCACAAGCCUC | FIG. 8 |
| crRNA- | GAUGUGGUAUGAU (SEQ ID NO: 22) | |
| At/Ah | | |
| 21a- | AAATGCCTTCGATTTTACCCCAACGAATAAGTACCATA (SEQ ID NO: 23) | FIG. 5, 11A, |
| PAMv1 ※ | | C, 15D, F, 2B, |
| | | 6A, 7A, C, 9, |
| | | 10, |
| 21a- | AAATGCCTTCGATTTTACCCCAACGAATAAGTACGATA (SEQ ID NO: 24) | FIG. 5E, 9 |
| PAMv2 ※ | | |
| 21a- | AAATGCCTTCGATTTTACCCCAACGAATAAGTCATATA (SEQ ID NO: 25) | FIG. 5A, 11A, |
| PAMv3 ※ | | C, D, 7A, 10, |
| 21a- | AAATGCCTTCGATTTTACCCCAACGAATAAGTACCGGT (SEQ ID NO: 26) | FIG. 5E, F, G, |
| PAMv4 ※ | | 15F, 9 |
| 21a- | AAATGCCTTCGATTTTACCCCAACGAATAAGTACTAGT (SEQ ID NO: 27) | FIG. 5E, F, G, |
| PAMv5 ※ | | 9 |
| 21a- | AAATGCCTTCGATTTTACCCCAACGAATAAGTACCAGG (SEQ ID NO: 28) | FIG. 5C, D, E, |
| PAMv6 ※ | | 15F, 7C |
| 21a- | AAATGCCTTCGATTTTACCCCAACGAATAAGTACAAGA (SEQ ID NO: 29) | FIG. 5E, 9 |
| PAMv7 ※ | | |
| EGFP ※ | GTTGTGGCTGTTGTAGTTGTACTCCAGCTTGTGCCCCA (SEQ ID NO: 30) | FIG. 5F, G |
| yz101- | CCTTATCCGACCAACATATCATAACGGAGTGATCGCATTGGAGCTCTGGTTTG | FIG. 5F, G |
| LHA- | TGGGGCA (SEQ ID NO: 31) | |
| lacA-F | | |
| yz102- | TAGGCCTGATAAGCGCAGCGTATCAGGCAATTTTTATAATCCGGGAAGCCCTG | FIG. 5F, G |
| LacA- | GGCCAAC (SEQ ID NO: 32) | |
| LHA-R | | |
| 50nt | CAGGTCCATATTTATATGACTTATTCGTTGGGGTAAAATCGAAGGCATTT | FIG. 11C, 15G |
| 21aPAM3- | (SEQ ID NO: 33) | |
| TS | | |
| 0 bulge | AAATGCCTTCGATTTTACCCCAACGAATAAGTCATATAAATATGGACCTG | FIG. 11C, 15G |
| 50nt | (SEQ ID NO: 34) | |
| 21aPAM3- | | |
| NTS | | |
| 1-2 bulge | AAATGCCTTCGATTTTACCCCAACGAATttGTCATATAAATATGGACCTG | FIG. 11C, 15G |
| 50nt | (SEQ ID NO: 35) | |
| 21aPAM3- | | |
| NTS | | |
| 3-4 bulge | AAATGCCTTCGATTTTACCCCAACGAtaAAGTCATATAAATATGGACCTG | FIG. 11C, 15G |
| 50nt | (SEQ ID NO: 36) | |
| 21aPAM3- | | |
| NTS | | |
| 5-6 bulge | AAATGCCTTCGATTTTACCCCAACctATAAGTCATATAAATATGGACCTG | FIG. 11C |
| 50nt | (SEQ ID NO: 37) | |
| 21aPAM3- | | |
| NTS | | |
| 19-20 | AAATGCCTTCctTTTTACCCCAACGAATAAGTCATATAAATATGGACCTG | FIG. 11C |
| bulge | (SEQ ID NO: 38) | |
| 50nt | | |
| 21aPAM3- | | |
| NTS | | |
| 21-22 | AAATGCCTagGATTTTACCCCAACGAATAAGTCATATAAATATGGACCTG | FIG. 11C |
| bulge | (SEQ ID NO:39) | |
| 50nt | | |
| 21aPAM3- | | |
| NTS | | |
| 23-25 | AAATGggaTCGATTTTACCCCAACGAATAAGTCATATAAATATGGACCTG | FIG. 11C |
| bulge | (SEQ ID NO: 40) | |
| 50nt | | |
| 21aPAM3- | | |
| NTS | | |
| ctrl bulge | AAATGCCTTCGATTTTACCCCAACGAATAAGTCATATAAATATccACCTG | FIG. 11C, 15G |
| 50nt | (SEQ ID NO: 41) | |
| 21aPAM3- | | |
| NTS | | |
| 50nt | CAGGTCCATATTTTAGCCTGTTATTCGTTGGGGTAAAATCGAAGGCATTT | FIG. 11C, |
| 21aPAM- | (SEQ ID NO: 42) | 15G, 17A, |
| TS | | 14A, B |
| 0 bulge | AAATGCCTTCGATTTTACCCCAACGAATAACAGGCTAAAATATGGACCTG | FIG. 11C, |
| 50nt | (SEQ ID NO: 43) | 15G, 17A, |
| 21aPAM- | | 14A, B |
| NTS | | |
| 1-2 bulge | AAATGCCTTCGATTTTACCCCAACGAATttCAGGCTAAAATATGGACCTG | FIG. 11C, 15G |
| 50nt | (SEQ ID NO: 44) | |
| 21aPAM- | | |
| NTS | | |
| 120 nt | AGGTTTAAACGAATTCGCCCTTGGTCCATATTTTAGCCTGTATATGACTTATT | FIG. 11D, 12 |
| 21aPAM3- | CGTTGGGGTAAAATCGAAGGCATTTGTGCACCAATTTCTGATGAAGGGCGAAT | |
| TS | TCGCGGCCGCTAAA (SEQ ID NO: 45) | |
| 0 bulge | TTTAGCGGCCGCGAATTCGCCCTTCATCAGAAATTGGTGCACAAATGCCTTCG | FIG. 11D, 12 |
| 120 nt | ATTTTACCCCAACGAATAAGTCATATACAGGCTAAAATATGGACCAAGGGCGA | |
| 21aPAM3- | ATTCGTTTAAACCT (SEQ ID NO: 46) | |
| NTS | | |
| 1-2 bulge | TTTAGCGGCCGCGAATTCGCCCTTCATCAGAAATTGGTGCACAAATGCCTTCG | FIG. 11D, 12 |
| 120 nt | ATTTTACCCCAACGAATttGTCATATACAGGCTAAAATATGGACCAAGGGCGA | |
| 21aPAM3- | ATTCGTTTAAACCT (SEQ ID NO: 47) | |
| NTS | | |
| 3-4 bulge | TTTAGCGGCCGCGAATTCGCCCTTCATCAGAAATTGGTGCACAAATGCCTTCG | FIG. 11D, 12 |
| 120 nt | ATTTTACCCCAACGAtaAAGTCATATACAGGCTAAAATATGGACCAAGGGCGA | |
| 21aPAM3- | ATTCGTTTAAACCT (SEQ ID NO: 48) | |
| NTS | | |
| 5-6 bulge | TTTAGCGGCCGCGAATTCGCCCTTCATCAGAAATTGGTGCACAAATGCCTTCG | FIG. 11D, 12 |
| 120 nt | ATTTTACCCCAACctATAAGTCATATACAGGCTAAAATATGGACCAAGGGCGA | |
| 21aPAM3- | ATTCGTTTAAACCT (SEQ ID NO: 49) | |
| NTS | | |
| 19-20 | TTTAGCGGCCGCGAATTCGCCCTTCATCAGAAATTGGTGCACAAATGCCTTCc | FIG. 11D, 12 |
| bulge | tTTTTACCCCAACGAATAAGTCATATACAGGCTAAAATATGGACCAAGGGCGA | |
| 120 nt | ATTCGTTTAAACCT (SEQ ID NO: 50) | |
| 21aPAM3- | | |
| NTS | | |
| 21-22 | TTTAGCGGCCGCGAATTCGCCCTTCATCAGAAATTGGTGCACAAATGCCTagG | FIG. 11D, 12 |
| bulge | ATTTTACCCCAACGAATAAGTCATATACAGGCTAAAATATGGACCAAGGGCGA | |
| 120 nt | ATTCGTTTAAACCT (SEQ ID NO: 51) | |
| 21aPAM3- | | |
| NTS | | |
| 23-25 | TTTAGCGGCCGCGAATTCGCCCTTCATCAGAAATTGGTGCACAAATGggaTCG | FIG. 11D, 12 |
| bulge | ATTTTACCCCAACGAATAAGTCATATACAGGCTAAAATATGGACCAAGGGCGA | |
| 120 nt | ATTCGTTTAAACCT (SEQ ID NO: 52) | |
| 21aPAM3- | | |
| NTS | | |
| ctrl bulge | TTTAGCGGCCGCGAATTCGCCCTTCATCAGAAATTGGTGCACAAATGCCTTCG | FIG. 11D, 12 |
| 120 nt | ATTTTACCCCAACGAATAAGTCATATACAGGCTAAAATATGGAggAAGGGCGA | |
| 21aPAM3- | ATTCGTTTAAACCT (SEQ ID NO: 53) | |
| NTS | | |
| 1-F89 | GATCGAGAACTTTCGAACCCTTATACCATGAACGATCGCCATTTCGAATGTGT | FIG. 11E, 13 |
| | GTACGCGCGTAAATAGGTCAATGTGTACGTATGTGC (SEQ ID NO: 54) | |
| 1-R89 | GCACATACGTACACATTGACCTATTTACGCGCGTACACACATTCGAAATGGCG | FIG. 11E, 13 |
| | ATCGTTCATGGTATAAGGGTTCGAAAGTTCTCGATC (SEQ ID NO: 55) | |
| Z6, target | GATCGCACATACGTACACATTGACCTATTTACG (SEQ ID NO: 56) | FIG. 11E |
| sequence | | |
| Z7, target | ACATACGTACACATTGACCTATTTACGCGCGTA (SEQ ID NO: 57) | FIG. 11E |
| sequence | | |
| 27m-wt | GAAGATCCTTTGATCTTTTCTACGGGGTCCTAA (SEQ ID NO: 58) | FIG. 15B |
| PAM | | |
| Target | | |
| sequence | | |
| in mini | | |
| plasmid | | |
| 27m-mut | GAAGATCCTTTGATCTTTTCTACGGGGTCTGAC (SEQ ID NO: 59) | FIG. 15B |
| PAM | | |
| Target | | |
| sequence | | |
| in mini | | |
| plasmid | | |
| 21a-wt | AAATGCCTTCGATTTTACCCCAACGAATAACAGGCTAA (SEQ ID NO: 60) | FIG. 176, 1C, |
| PAM | | 8, 18 |
| Target | | |
| sequence | | |
| in 1612 bp | | |
| dsDNA | | |
| SV40 NLS | CCAAAGAAGAAGCGGAAGGTC (SEQ ID NO: 61) | FIG. 17C |
| c-myc | CCAGCAGCCAAGAGGGTGAAGCTGGAT (SEQ ID NO: 62) | FIG. 17C |
| NLS | | |
| nucleoplas | AAAAGGCCGGCGGCCACGAAAAAGGCCGGCCAGGCAAAAAAGAAAAAG (SEQ | FIG. 17c |
| min NLS | ID NO: 63) | |
| 6m-mut | CATGAGACAATAACCCTGATAAATGCTTCAATAATAGC (SEQ ID NO: 64) | FIG. 6B |
| PAM Δ | | |
| 16m-mut | TGAATGTCAGCTACTGGGCTATCTGGACAAGGGAAAAC (SEQ ID NO: 65) | FIG. 6B |
| PAM Δ | | |
| 18m-mut | ACGGCTCTCTCTTTTATAGGTGTAAACCTTAAACTGCC (SEQ ID NO: 66) | FIG. 6B |
| PAM Δ | | |
| 24m-mut | ACCTGTAGACAGCATGAAGTTAGGATTGTGAAAGAGTT (SEQ ID NO: 67) | FIG. 6B |
| PAM Δ | | |
| 28m-mut | CGCCATTAACCTGATGTTCTGGGGAATATAAATGTCAG (SEQ ID NO: 68) | FIG. 6B |
| PAM Δ | | |
| EMX1 | GATGTTGAGCTCCGTTTGGGGGTATGGTGAGTGTCTAG (SEQ ID NO: 69) | FIG. 8 |
| Target | | |
| sequence | | |
| in 1504 bp | | |
| dsDNA | | |
| EMX1-1 | TCACAGGTGCCTCTTAGCTGCTAAGCCTGG | FIG. 17 |
| crRNA- | GUCAUAGUUCCCUCACAAGCCUCGAUGUGGUAUGAU (SEQ ID NO: 75) | |
| At/Ah | | |
| EMX1-3 | GATGTTGAGCTCCGTTTGGGGGTATGGTGA | FIG. 17 |
| crRNA- | GUCAUAGUUCCCUCACAAGCCUCGAUGUGGUAUGAU (SEQ ID NO : 76) | |
| At/Ah | | |
| EMX1 | AGGCTGGAGTATAGTGATTTGATCATGGCTGACTGCAGGCTTGAACTTTTAGG | |
| substrate | CTCAAGTGATTCTCCCACCTCAGCTTCCAGAGTAGCTGGGACTACAGGCATGC | |
| | ACCACCACACCTGGCTAATATTTTTAATTTTTTGTAGAGACAGGGTCTCGCTA | |
| | TGTTGCCCAGGCTGGTCTCGAACTCCTGGCCTCAAGCAGTCTTTCTGCCACAG | |
| | CCTCCCAAAGTGCTGAGATTATAGGTGTGAGCCATTGTACCTGGCTTGGAGTA | |
| | ATGTTTTTGAGCAAAGCAAATGTACAGGACATGCACAGAGAGGCACAGGGAGC | |
| | ACAGAGTCCCTGGATGAATGCTCTGGCTGGTGGCAGTATTGTCACAGGGCAGG | |
| | CTACCCAAAGGTGAGCAGAACACATGAGGCTAGACAGGTACAACTTGCTCATG | |
| | AAGAGCCTCGAATGCCAAGCTTAGGAGTTTGAACTTTATTGGAACACTGACAC | |
| | TAATAGTTAATGTTTACTGAGTGCTTACTATGTGCTAAGAGCTATGCATGAAT | |
| | CACCTCATTCTAATTCTCTTAACTCTCCACAGCAGGTCTTATTATTTCCAGAT | |
| | TGCTGATGAAAAACTGAGTCACAGAGAGGTAAAAAAAAAAAAACAAAAACACC | |
| | CTGGGTCCAGAGTTGTGTGAAAAAATTTTAACAGGTTAGAGTTGTGTTGTAGA | |
| | AATCTGGTAGCCAGCCACGTGTAAGACGAATTGTAGAAAGGAGAGATCACAAA | |
| | TGGCTATCATAAGAGAATCACTGGGAAAAGGTAATAAGAACCCAAATTAGAGG | |
| | CAAGGAGAACAGAAAGAAATGAAAGAATGCAAAGACTGACTGGATGTGCACAG | |
| | CAGTGACCGAGAGAAAGGAGGTATCACAGGTGCCTCTTAGCTGCTAAGCCTGG | |
| | GTGTCCTGCAGAAAGGGGCAGCTATTATCAGAAGAGAGAAGCCAGAGAGAGGC | |
| | ACAGATTTGGTGGGGATGATGTTGAGCTCCGTTTGGGGGTATGGTGAGTGTCT | |
| | AGGGGGCCTGTAGGAACCCCTCCAGAAAAATTCTCACAAGCATTTGAAAATCA | |
| | GTGACTTGATCTGGAGAAAAATATAGGGCTGGCATTACAAACCTGCCGGGGTC | |
| | TGCATTACAGTATTGGTTGAAAGTGTGGAAATTGATAATGATAGATGGTTGGT | |
| | AGATACATAGATAGATAGATAGATAGATAGATAGATAGATAGATAGATAGATA | |
| | GATAGATAGAATAAATAGCATGATACATGGAATGAGGGAAGGGCTAAGGATAA | |
| | AGATGCCTTCATGGATGCCCACAGTTAGGAGAAGAAAGTAGAGCCCATGAAGG | |
| | AACAACTCCCAGAGTTTGCAGATCCAGGCAAGTATCTTGTTCAGAAAATAAAG | |
| | GGAAGGGAGTTTCCAAAAGAAAAGGGGGTTGAAGGTACTCAGATGCTGAGGAG | |
| | CCTATAAACAATTGTGACTAAAAAAGACTCAAATTTGGAAATAAGGTGTCCTA | |
| | ATGAACATTTCAGAGG (SEQ ID NO: 78) | |
| 21 | CTTCCAGGGGGTTGGTTGTTTATTTCTTCCAAATGGGTCATAAAAGGATGATA | |
| substrate | AAGCTGCAAACCCAAAGAAACTAATTATTCACTACTTGAAAGGATGAAGTGAA | |
| | TTTTGAGCTTTATGAATGCAAACTAATAATAAGTATCGTGGATAGTAATTATG | |
| | CTTACAATTTTAATTAGTGAATGCTGTTGTAAATTATAAGGCTGTGTCTACAT | |
| | TAGAAACAAAAAACACGGAACTCTAAACCTGCAACAAACCTGAGTGTACATAG | |
| | ACTGAGATTCTAAGGGACTTTTAAGATGAAAAATTCACATGGTTTCAGATAAA | |
| | ACTCACACAGGTCATCCTGAAAGTCACTGTTGGTCGATGGTCATTAGCACTAT | |
| | AGTTTGTAACATAATAAGAAGAGGTGGGAGTAATGTTTCTCTTCTGGTAAAAA | |
| | ACATTGGCAATTACATATGCCAGGAAACCCGACTGATTTGAGTTGTAATGCAC | |
| | AGATCGCTAGCGATGCAGAAAAAAAGTCAAACAATGCCATGTGAAGTGTACTC | |
| | TCTAGACCCAATTAGATCAATCTTTCCCAAATTACTTAGTACATGATTGGAAA | |
| | GAGAGCAGCTGTATTTTCAATCCATCATCCCATCCACCTATCTCAAATTTGCC | |
| | TGGGTTAATTCTTTTGACCCACCAGTTATATCAAGTAACATATGTTTTTCCCT | |
| | CAATTGATCAACTAACAAAATCGATGAGAATTTTTATAGTCACATAGTTATGG | |
| | AGACAAATTCTCAGATGAACATACTTTGAGCAGGGGTGGGATTAGGAGGTACG | |
| | GAGGGGCAAGGGTTAATGCTGACAGCTGTTACCAAAGCCTCTTTTCAAGTCCC | |
| | CGCTTTACTGAAGTTTGTTCTATCTTACATGCATCACACATCAGAAATTGGTG | |
| | CACAAATGCCTTCGATTTTACCCCAACGAATAACAGGCTAAAATATGGACCTG | |
| | TAGACAGCATGAAGTTAGGATTGTGAAAGAGTTATCTTGGATTCATATTTTGA | |
| | CACTCAGAAACCTTGCAAATGCTTGATCTCCTCTCTTCTAACAAAAGGTCTCA | |
| | GCAGTAGACCAGCCTCGGGATCTCAAAGGCTGAGTGGTTGGTGCTGGCTCAGC | |
| | CCTGTCCCCACCGGCCACTGCCATCTCGACATTGCCATCTGACATCAGAGCTA | |
| | CAGGCGTGCCTCCCTCCACACAGCACAGATTCATTTTGACAAACTAGTTTGGG | |
| | GTGGCTTGGACAGTGCAGGAACAGTAGTTTCTGGTGAGTGATATCATTAGTTC | |
| | AGGAGGCCAGGGTGCAGCAGCAGCAGCAGCCCCCCATTTCCATCCCAGCTGCC | |
| | CACCCTATACCCACACACATACACCCCGTTACTTGGCCCACAATCTTCCCTCC | |
| | TAACATCTTCAACTTAGTCTGTACACAATTTCCAAGTAATAACTGTGTGCTCA | |
| | AGTGTGACCTCTCATTATCACACCTGTTCCACTTATTCATTCACTCTGCAAAT | |
| | ACTATCGTCTGTTTTTTGCCAAACATCATGTATCAAAAATGTGACAAAATAAA | |
| | AAGCCTTTTTATAAGTCTCAAGTATCCACAAAATAACACTACAATTCTTGCTC | |
| | TTCCTTCAGACAACCTCTTGCA (SEQ ID NO: 79) | |
| SSDNA- | CAGGTCCATATTTTAGCCTGTTATTCGTTGGGGTAAAATCGAAGGCATTT | |
| 21-TS | (SEQ ID NO: 80) | |
| SSRNA-21- | CAGGUCCAUAUUUUAGCCUGUUAUUCGUUGGGGUAAAAUCGAAGGCAUUU | |
| TS | (SEQ ID NO: 81) | |
| AtCas9 | ATGGCATATCGACTGGGATTGGATATTGGCATTACATCCGTAGGGTGGGCCGT | |
| gene | TGTGGCACTTGAGAAGGATGAAAGCGGATTAAAACCCGTTCGCATTCAAGATC | |
| sequence | TTGGTGTTCGCATTTTTGATAAGGCTGAAGACAGTAAGACGGGGGCATCTTTG | |
| (orginal | GCTCTTCCCCGCCGTGAGGCGCGCAGTGCACGGCGTCGCACGCGCCGTCGTCG | |
| locus) | CCATCGGCTTTGGCGCGTAAAGCGTTTGTTGGAACAGCACGGGATTCTTTCGA | |
| | TGGAACAAATTGAAGCGCTTTATGCGCAACGAACGTCATCTCCTGATGTGTAT | |
| | GCTCTGCGTGTGGCTGGGCTCGATAGATGTCTCATCGCAGAGGAAATCGCGAG | |
| | AGTGCTTATTCATATTGCGCATCGTCGCGGTTTTCAGTCAAATAGGAAATCTG | |
| | AAATCAAAGATTCAGACGCCGGAAAGTTGCTGAAGGCTGTGCAGGAAAATGAA | |
| | AATTTAATGCAATCGAAGGGATACCGAACGGTTGCGGAAATGCTTGTTTCTGA | |
| | GGCAACGAAAACCGATGCTGAGGGAAAACTCGTTCATGGTAAGAAACACGGTT | |
| | ATGTATCGAATGTGAGAAACAAGGCAGGCGAGTATCGTCATACGGTCTCGCGG | |
| | CAAGCCATTGTGGACGAAGTTCGGAAGATTTTCGCAGCCCAACGAGCGCTGGG | |
| | TAATGATGTGATGAGTGAAGAGTTGGAGGATTCATATCTGAAGATTCTTTGCT | |
| | CTCAACGCAACTTCGATGATGGCCCAGGTGGAGACAGTCCTTATGGGCATGGT | |
| | TCTGTCTCGCCTGATGGCGTAAGGCAAAGTATTTATGAACGAATGGTAGGCTC | |
| | GTGTACGTTCGAGACAGGTGAAAAGCGGGCGCCGCGCAGTTCGTATTCGTTCG | |
| | AGCGGTTCCAGCTGCTTACCAAGGTCGTGAATCTTCGCATCTATCGACAACAG | |
| | GAGGATGGAGGAAGATATCCGTGTGAGCTCACACAGACAGAACGAGCACGCGT | |
| | GATTGATTGTGCGTATGAGCAAACGAAAATTACGTATGGGAAACTGCGGAAGC | |
| | TGCTAGACATGAAAGACACGGAAAGCTTCGCGGGACTTACGTATGGCTTGAAT | |
| | CGTAGCCGTAATAAAACGGAGGACACGGTCTTTGTCGAGATGAAGTTTTATCA | |
| | CGAGGTCAGAAAGGCTCTGCAGCGTGCTGGGGTTTTCATTCAAGATTTGTCCA | |
| | TTGAAACGCTTGATCAAATCGGTTGGATCTTGTCTGTGTGGAAAAGCGATGAC | |
| | AACCGCAGAAAGAAGCTTTCGACTCTCGGGTTGTCCGACAATGTAATTGAGGA | |
| | ATTACTACCGTTAAATGGTTCGAAATTTGGACACCTTTCGCTGAAGGCCATTC | |
| | GAAAAATACTTCCGTTCTTAGAGGATGGCTACAGCTATGATGTAGCCTGCGAG | |
| | CTGGCTGGGTATCAGTTTCAAGGGAAAACAGAGTACGTAAAACAGAGGCTTTT | |
| | GCCACCTTTGGGTGAAGGAGAAGTGACCAATCCGGTGGTGCGGCGTGCCCTTT | |
| | CACAGGCCATCAAAGTTGTGAATGCGGTCATCCGCAAGCATGGTTCGCCCGAA | |
| | TCTATTCACATCGAGCTTGCCCGGGAGCTATCCAAGAATCTGGATGAGCGGCG | |
| | TAAAATTGAGAAGGCTCAGAAGGAAAACCAAAAAAATAACGAGCAGATCAAGG | |
| | ACGAGATTCGTGAGATTCTTGGAAGTGCGCATGTTACCGGACGAGACATTGTG | |
| | AAGTACAAGCTATTCAAGCAGCAGCAGGAATTCTGCATGTATTCTGGGGAAAA | |
| | GTTGGACGTCACACGTCTATTCGAGCCAGGTTACGCCGAAGTGGATCACATCA | |
| | TCCCGTATGGGATTTCATTTGACGATAGCTATGACAACAAGGTTCTTGTCAAG | |
| | ACGGAGCAAAATCGTCAGAAGGGGAACCGAACGCCATTGGAGTATCTGCGAGA | |
| | TAAGCCAGAGCAGAAGGCAAAGTTTATAGCGTTGGTAGAAAGTATCCCATTGT | |
| | CGCAAAAGAAGAAGAACCATTTGTTAATGGACAAGCGCGCGATCGATTTGGAA | |
| | CAAGAAGGATTTCGCGAGAGAAATCTTTCGGACACGCGGTACATCACCAGGGC | |
| | TTTGATGAATCACATTCAGGCATGGTTGTTATTTGACGAAACGGCTTCCACGC | |
| | GGTCAAAGCGTGTGGTTTGTGTCAATGGCGCCGTTACGGCATATATGCGAGCG | |
| | CGGTGGGGATTGACGAAGGATCGGAATGCCGGTGATAAGCACCATGCGGCCGA | |
| | TGCAGTTGTTGTTGCGTGCATCGGAGATTCGCTTATTCAACGCGTCACGAAGT | |
| | ATGACAAATTTAAGCGCAATGCGCTCGCCGATCGCAACCGGTATGTGCAGCAG | |
| | GTCAGCAAAAGCGAAGGCATCACGCAATATGTGGATAAAGAGACGGGTGAGGT | |
| | GTTCACGTGGGAATCATTTGACGAACGCAAGTTTCTCCCAAACGAACCTTTGG | |
| | AGCCCTGGCCATTCTTTCGAGACGAGCTGTTGGCAAGACTCAGCGACGATCCT | |
| | AGCAAAAATATACGAGCTATTGGCCTCTTAACCTATTCGGAAACCGAGCAAAT | |
| | TGATCCTATCTTTGTCTCGAGAATGCCGACAAGGAAGGTCACGGGCGCGGCTC | |
| | ACAAGGAGACGATTCGTAGCCCGCGTATCGTGAAAGTCGATGATAATAAGGGC | |
| | ACAGAGATTCAGGTCGTGGTTTCAAAGGTGGCTCTTACCGAACTCAAGCTCAC | |
| | AAAGGATGGGGAGATTAAGGATTATTTCCGACCGGAAGATGATCCTAGATTGT | |
| | ACAACACTCTGCGCGAAAGGCTTGTTCAATTTGGCGGCGATGCGAAGGCTGCT | |
| | TTTAAAGAACCTGTTTACAAGATTTCGAAGGACGGTAGTGTGCGAACACCCGT | |
| | TAGGAAGGTCAAGATTCAAGAAAAGTTGACGCTCGGTGTACCTGTTCATGGCG | |
| | GACGAGGCATCGCAGAGAATGGGGGAATGGTGAGAATTGATGTGTTTGCAAAA | |
| | GACGGAAAGTATTACTTTGTACCGATTTATGTCGCAGATGTACTGAAGCGGGA | |
| | ACTTCCGAATCGCCTAGCCACTGCGCACAAGCCGTACTCCGAGTGGAGAGTTG | |
| | TGGATGATAGCTATCAATTTAAATTTAGCTTATATCCAAACGACGCGGTGATG | |
| | ATTAAACCTAGTAGGGAAGTGGATATAACTTATAAGGACAGGAAGGAACCTGT | |
| | TGGATTACGACTGATGTATTTTGCGGGTGCGGATATTTCAGGCGCTACTTTAA | |
| | CTCTGCGTACGCACGACAATTCTGGAGAACTGAAGGGTTTGGGTATACAAGGT | |
| | CTAGAGGTGTTCGAAAAATACGTTGTTGGTCCGTTAGGAGACACGCATCCTGT | |
| | TTATAAGGAACGGCGCATGCCATTTCGTGTGGAAAGGAAGATGAATTGA | |
| | (SEQ ID NO: 82) | |
| Codon- | ATGGCCTACAGACTGGGCCTGGACATCGGCATCACCTCCGTGGGATGGGCAGT | |
| optimized | GGTGGCCCTGGAGAAGGACGAGTCTGGCCTGAAGCCCGTGCGGATCCAGGATC | |
| AtCas9 | TGGGCGTGCGCATCTTTGACAAGGCCGAGGATAGCAAGACCGGCGCCTCCCTG | |
| gene | GCCCTGCCTAGGAGAGAGGCACGGAGCGCCCGGCGCAGGACAAGACGGCGCAG | |
| | GCACAGGCTGTGGAGGGTGAAGAGACTGCTGGAGCAGCACGGCATCCTGTCCA | |
| | TGGAGCAGATCGAGGCCCTGTACGCACAGCGCACCAGCTCCCCAGACGTGTAT | |
| | GCACTGAGGGTGGCAGGACTGGATAGGTGCCTGATCGCCGAGGAGATCGCCAG | |
| | GGTGCTGATCCACATCGCCCACAGACGGGGCTTCCAGAGCAACAGAAAGTCCG | |
| | AGATCAAGGACAGCGATGCCGGCAAGCTGCTGAAGGCCGTGCAGGAGAACGAG | |
| | AATCTGATGCAGTCCAAGGGCTACCGGACAGTGGCCGAGATGCTGGTGTCTGA | |
| | GGCCACCAAGACAGACGCCGAGGGCAAGCTGGTGCACGGCAAGAAGCACGGCT | |
| | ACGTGAGCAACGTGCGCAATAAGGCAGGAGAGTATAGGCACACCGTGTCCAGG | |
| | CAGGCAATCGTGGATGAGGTGAGGAAGATCTTTGCAGCACAGAGAGCCCTGGG | |
| | AAACGACGTGATGTCCGAGGAGCTGGAGGATTCTTACCTGAAGATCCTGTGCA | |
| | GCCAGAGGAATTTCGACGATGGACCAGGAGGCGACTCCCCATACGGACACGGA | |
| | TCCGTGTCTCCTGATGGCGTGAGGCAGTCCATCTATGAGAGAATGGTGGGCTC | |
| | TTGTACCTTCGAGACAGGAGAGAAGAGGGCACCACGCTCTAGCTATTCCTTCG | |
| | AGCGGTTTCAGCTGCTGACCAAGGTGGTGAACCTGAGGATCTACAGACAGCAG | |
| | GAGGACGGCGGCCGCTATCCTTGCGAGCTGACCCAGACAGAGCGCGCCAGAGT | |
| | GATCGATTGTGCCTACGAGCAGACCAAGATCACATATGGCAAGCTGAGAAAGC | |
| | TGCTGGACATGAAGGATACCGAGAGCTTTGCCGGCCTGACATACGGCCTGAAC | |
| | AGATCCCGGAATAAGACCGAGGACACAGTGTTCGTGGAGATGAAGTTTTATCA | |
| | CGAGGTGAGGAAGGCCCTGCAGAGAGCCGGCGTGTTCATCCAGGACCTGAGCA | |
| | TCGAGACCCTGGATCAGATCGGCTGGATCCTGAGCGTGTGGAAGAGCGACGAT | |
| | AACCGCAGGAAGAAGCTGTCTACACTGGGCCTGAGCGACAACGTGATCGAGGA | |
| | GCTGCTGCCCCTGAATGGCAGCAAGTTTGGCCACCTGTCCCTGAAGGCCATCA | |
| | GGAAGATCCTGCCTTTCCTGGAGGACGGCTACTCCTATGATGTGGCCTGCGAG | |
| | CTGGCCGGCTACCAGTTCCAGGGCAAGACCGAGTATGTGAAGCAGAGACTGCT | |
| | GCCACCTCTGGGAGAGGGAGAGGTGACAAACCCAGTGGTGAGACGGGCCCTGT | |
| | CCCAGGCCATCAAGGTGGTGAATGCCGTGATCCGGAAGCACGGCTCTCCCGAG | |
| | AGCATCCACATCGAGCTGGCCCGCGAGCTGTCTAAGAACCTGGACGAGCGCAG | |
| | GAAGATCGAGAAGGCCCAGAAGGAGAATCAGAAGAACAATGAGCAGATCAAGG | |
| | ACGAGATCAGGGAGATCCTGGGATCCGCCCACGTGACCGGAAGAGATATCGTG | |
| | AAGTACAAGCTGTTTAAGCAGCAGCAGGAGTTCTGTATGTATTCTGGCGAGAA | |
| | GCTGGACGTGACACGGCTGTTTGAGCCAGGCTACGCCGAGGTGGATCACATCA | |
| | TCCCCTACGGCATCTCCTTCGACGATTCTTATGACAACAAGGTGCTGGTGAAA | |
| | ACCGAGCAGAACCGGCAGAAGGGCAATCGCACACCACTGGAGTATCTGCGGGA | |
| | TAAGCCCGAGCAGAAGGCCAAGTTTATCGCCCTGGTGGAGAGCATCCCCCTGT | |
| | CCCAGAAGAAGAAGAACCACCTGCTGATGGACAAGCGCGCCATCGATCTGGAG | |
| | CAGGAGGGCTTTCGCGAGAGGAACCTGAGCGACACCAGGTACATCACAAGAGC | |
| | CCTGATGAATCACATCCAGGCCTGGCTGCTGTTCGATGAGACCGCCTCCACAC | |
| | GGTCTAAGAGGGTGGTGTGCGTGAACGGAGCAGTGACCGCCTATATGAGAGCC | |
| | CGGTGGGGCCTGACAAAGGACAGGAATGCAGGCGATAAGCACCACGCAGCAGA | |
| | CGCAGTGGTGGTGGCCTGTATCGGCGATAGCCTGATCCAGAGGGTGACCAAGT | |
| | ACGACAAGTTCAAGAGAAACGCCCTGGCCGATCGCAATAGATACGTGCAGCAG | |
| | GTGAGCAAGTCCGAGGGCATCACACAGTATGTGGACAAGGAGACCGGCGAGGT | |
| | GTTCACATGGGAGTCCTTTGATGAGAGGAAGTTCCTGCCTAACGAGCCACTGG | |
| | AGCCATGGCCCTTCTTTAGGGACGAGCTGCTGGCAAGGCTGTCTGACGATCCT | |
| | AGCAAGAATATCAGAGCCATCGGCCTGCTGACCTACTCTGAGACAGAGCAGAT | |
| | CGATCCCATCTTTGTGAGCCGGATGCCTACCAGGAAGGTGACAGGAGCAGCAC | |
| | ACAAGGAGACCATCAGGTCTCCAAGAATCGTGAAGGTGGACGATAACAAGGGC | |
| | ACAGAGATCCAGGTGGTGGTGAGCAAGGTGGCCCTGACCGAGCTGAAGCTGAC | |
| | AAAGGACGGCGAGATCAAGGATTACTTCAGGCCCGAGGACGATCCTAGACTGT | |
| | ATAATACCCTGAGAGAGCGGCTGGTGCAGTTTGGAGGCGACGCAAAGGCAGCC | |
| | TTCAAGGAGCCCGTGTACAAGATCTCCAAGGATGGCTCTGTGAGGACCCCAGT | |
| | GAGAAAGGTGAAGATCCAGGAGAAGCTGACACTGGGAGTGCCAGTGCACGGAG | |
| | GAAGGGGAATCGCCGAGAACGGCGGCATGGTGCGCATCGACGTGTTTGCCAAG | |
| | GATGGCAAGTACTATTTCGTGCCCATCTACGTGGCCGACGTGCTGAAGAGGGA | |
| | GCTGCCAAACAGACTGGCAACCGCACACAAGCCTTATTCTGAGTGGAGGGTGG | |
| | TGGACGATTCTTACCAGTTCAAGTTTAGCCTGTATCCTAATGATGCCGTGATG | |
| | ATCAAGCCAAGCAGAGAGGTGGACATCACCTACAAGGATAGGAAGGAGCCAGT | |
| | GGGACTGAGGCTGATGTATTTCGCAGGAGCAGACATCTCTGGAGCCACCCTGA | |
| | CACTGCGGACACACGATAACAGCGGAGAGCTGAAGGGACTGGGAATCCAGGGC | |
| | CTGGAGGTGTTTGAGAAGTACGTGGTGGGACCTCTGGGCGACACCCACCCAGT | |
| | GTATAAGGAGAGACGGATGCCTTTCCGGGTGGAGCGCAAGATGAATTGA | |
| | (SEQ ID NO: 83) | |
| AtCas9 | MAYRLGLDIGITSVGWAVVALEKDESGLKPVRIQDLGVRIFDKAEDSKTGASL | |
| protein | ALPRREARSARRRTRRRRHRLWRVKRLLEQHGILSMEQIEALYAQRTSSPDVY | |
| sequence | ALRVAGLDRCLIAEEIARVLIHIAHRRGFQSNRKSEIKDSDAGKLLKAVQENE | |
| | NLMQSKGYRTVAEMLVSEATKTDAEGKLVHGKKHGYVSNVRNKAGEYRHTVSR | |
| | QAIVDEVRKIFAAQRALGNDVMSEELEDSYLKILCSQRNFDDGPGGDSPYGHG | |
| | SVSPDGVRQSIYERMVGSCTFETGEKRAPRSSYSFERFQLLTKVVNLRIYRQQ | |
| | EDGGRYPCELTQTERARVIDCAYEQTKITYGKLRKLLDMKDTESFAGLTYGLN | |
| | RSRNKTEDTVFVEMKFYHEVRKALQRAGVFIQDLSIETLDQIGWILSVWKSDD | |
| | NRRKKLSTLGLSDNVIEELLPLNGSKFGHLSLKAIRKILPFLEDGYSYDVACE | |
| | LAGYQFQGKTEYVKQRLLPPLGEGEVTNPVVRRALSQAIKVVNAVIRKHGSPE | |
| | SIHIELARELSKNLDERRKIEKAQKENQKNNEQIKDEIREILGSAHVTGRDIV | |
| | KYKLFKQQQEFCMYSGEKLDVTRLFEPGYAEVDHIIPYGISFDDSYDNKVLVK | |
| | TEQNRQKGNRTPLEYLRDKPEQKAKFIALVESIPLSQKKKNHLLMDKRAIDLE | |
| | QEGFRERNLSDTRYITRALMNHIQAWLLFDETASTRSKRVVCVNGAVTAYMRA | |
| | RWGLTKDRNAGDKHHAADAVVVACIGDSLIQRVTKYDKFKRNALADRNRYVQQ | |
| | VSKSEGITQYVDKETGEVFTWESFDERKFLPNEPLEPWPFFRDELLARLSDDP | |
| | SKNIRAIGLLTYSETEQIDPIFVSRMPTRKVTGAAHKETIRSPRIVKVDDNKG | |
| | TEIQVVVSKVALTELKLTKDGEIKDYFRPEDDPRLYNTLRERLVQFGGDAKAA | |
| | FKEPVYKISKDGSVRTPVRKVKIQEKLTLGVPVHGGRGIAENGGMVRIDVFAK | |
| | DGKYYFVPIYVADVLKRELPNRLATAHKPYSEWRVVDDSYQFKFSLYPNDAVM | |
| | IKPSREVDITYKDRKEPVGLRLMYFAGADISGATLTLRTHDNSGELKGLGIQG | |
| | LEVFEKYVVGPLGDTHPVYKERRMPFRVERKMN (SEQ ID NO: 84) | |
| AhCas9 | MAYRLGLDIGITSVGWAVVALEKDESGLKPIRIQDLGVRIFDKAEDSKTGASL | |
| protein | ALPRREARSARRRTRRRRHRLWRVKRLLEQHGILSMEQIEALYAQRTSSPDVY | |
| sequence | ALRVAGLDRCFTAEEIARVLIHIAHRRGFQSNRKSEIKDSDAGKLLKAVQENE | |
| | NLMQSKGYRTVAEMLVSEATKTDAEGKLVHGKKHGYVSNVRNKEGEYRHTVSR | |
| | QAIVDEVRKIFAAQRALGNDVMSEELEDSYLKILRSQRNEDDGPGGDSPYGHG | |
| | SVSPDGVRQSIYERMVGSCTFETGEKRAPRSSYSFERFQLLTKVVNLRIYRQQ | |
| | EDGGRYPCELTQKERARVIDCAYEQTKITYGKLRKLLDLKDTESFAGLTYGLN | |
| | RSRNKTEDTVFVEMKFYHEVRKALQRAGVFIQDLSIETLDQIGWILSVWKSDD | |
| | NRRKKLSTLGLSDNVIEELLPLNGSKFGHLSLKAIRKILPFLEDGYSYDVACE | |
| | LAGYQFQGKTEYVKQRLLPPLGEGEVTNPVVRRALSQAIKVVNAVIRKHGSPE | |
| | SIHIELARELSKNLDERRKIEKAQKENQKNNEQIKDEIREILGSAHVTGRDIV | |
| | KYKLFKQQQEFCMYSGEKLDVTRLFEPGYAEVDHIIPYGISFDDSYDNKVLVK | |
| | TEQNRQKGNRTPLEYLRDKPEQKAKFIALVESIPLSQKKKNHLLMDKRAIDLE | |
| | QEGFRERNLSDTRYITRALMNHIQAWLLFDETASMRSKRVVCVNGAVTAYMRA | |
| | RWGLTKDRDAGDKHHAADAVVVACIGDSLIQRVTKYDKFKRNALADRNRYVQQ | |
| | VSKSEGITQYVDKETGEVFTWESFDERKFLPNEPLEPWPFFRDELLARLSDDP | |
| | SKNIRAIGLLTYSETEQIDPIFVSRMPTRKVTGAAHKETIRSPRIVKVDDNKG | |
| | TEIQVVVSKVALTELKLTKDGEIKDYFRPEDDPRLYNTLRERLVQFGGDAKAA | |
| | FKEPVYKISKDGSVRTPVRKVKIQEKLTLGVPVHGGRGIAENGGMVRIDVFEK | |
| | GGKYYFVPIYVADVLKRELPNRLATAHKPYSEWRVVDDSYQFKFSLYPNDAVM | |
| | IKPRKEVDITYKDRKEPVGCRIMYFVSANIASASISLRTHDNSGELEGLGIQG | |
| | LEVFEKYVVDPLGDTHPVYKERRMPFRVERKMN (SEQ ID NO: 85) | |
| nucleoplas | AAAAGGCCGGCGGCCACGAAAAAGGCCGGCCAGGCAAAAAAGAAAAAG | |
| min NLS | (SEQ ID NO: 86) | |
| cmyc-NLS | Ccagcagccaagagggtgaagctggat (SEQ ID NO: 87) | |
| SV40NLS | CCAAAGAAGAAGCGGAAGGTC (SEQ ID NO: 88) | |
| NLS- | atgccagcagccaagagggtgaagctggatccagcagccaagagggtgaagct | |
| AtCas9- | ggatGGTATCCACGGAGTCCCAGCAGCCGCCTACAGACTGGGCCTGGACATCG | |
| NLS | GCATCACCTCCGTGGGATGGGCAGTGGTGGCCCTGGAGAAGGACGAGTCTGGC | |
| complete | CTGAAGCCCGTGCGGATCCAGGATCTGGGCGTGCGCATCTTTGACAAGGCCGA | |
| sequence | GGATAGCAAGACCGGCGCCTCCCTGGCCCTGCCTAGGAGAGAGGCACGGAGCG | |
| | CCCGGCGCAGGACAAGACGGCGCAGGCACAGGCTGTGGAGGGTGAAGAGACTG | |
| | CTGGAGCAGCACGGCATCCTGTCCATGGAGCAGATCGAGGCCCTGTACGCACA | |
| | GCGCACCAGCTCCCCAGACGTGTATGCACTGAGGGTGGCAGGACTGGATAGGT | |
| | GCCTGATCGCCGAGGAGATCGCCAGGGTGCTGATCCACATCGCCCACAGACGG | |
| | GGCTTCCAGAGCAACAGAAAGTCCGAGATCAAGGACAGCGATGCCGGCAAGCT | |
| | GCTGAAGGCCGTGCAGGAGAACGAGAATCTGATGCAGTCCAAGGGCTACCGGA | |
| | CAGTGGCCGAGATGCTGGTGTCTGAGGCCACCAAGACAGACGCCGAGGGCAAG | |
| | CTGGTGCACGGCAAGAAGCACGGCTACGTGAGCAACGTGCGCAATAAGGCAGG | |
| | AGAGTATAGGCACACCGTGTCCAGGCAGGCAATCGTGGATGAGGTGAGGAAGA | |
| | TCTTTGCAGCACAGAGAGCCCTGGGAAACGACGTGATGTCCGAGGAGCTGGAG | |
| | GATTCTTACCTGAAGATCCTGTGCAGCCAGAGGAATTTCGACGATGGACCAGG | |
| | AGGCGACTCCCCATACGGACACGGATCCGTGTCTCCTGATGGCGTGAGGCAGT | |
| | CCATCTATGAGAGAATGGTGGGCTCTTGTACCTTCGAGACAGGAGAGAAGAGG | |
| | GCACCACGCTCTAGCTATTCCTTCGAGCGGTTTCAGCTGCTGACCAAGGTGGT | |
| | GAACCTGAGGATCTACAGACAGCAGGAGGACGGCGGCCGCTATCCTTGCGAGC | |
| | TGACCCAGACAGAGCGCGCCAGAGTGATCGATTGTGCCTACGAGCAGACCAAG | |
| | ATCACATATGGCAAGCTGAGAAAGCTGCTGGACATGAAGGATACCGAGAGCTT | |
| | TGCCGGCCTGACATACGGCCTGAACAGATCCCGGAATAAGACCGAGGACACAG | |
| | TGTTCGTGGAGATGAAGTTTTATCACGAGGTGAGGAAGGCCCTGCAGAGAGCC | |
| | GGCGTGTTCATCCAGGACCTGAGCATCGAGACCCTGGATCAGATCGGCTGGAT | |
| | CCTGAGCGTGTGGAAGAGCGACGATAACCGCAGGAAGAAGCTGTCTACACTGG | |
| | GCCTGAGCGACAACGTGATCGAGGAGCTGCTGCCCCTGAATGGCAGCAAGTTT | |
| | GGCCACCTGTCCCTGAAGGCCATCAGGAAGATCCTGCCTTTCCTGGAGGACGG | |
| | CTACTCCTATGATGTGGCCTGCGAGCTGGCCGGCTACCAGTTCCAGGGCAAGA | |
| | CCGAGTATGTGAAGCAGAGACTGCTGCCACCTCTGGGAGAGGGAGAGGTGACA | |
| | AACCCAGTGGTGAGACGGGCCCTGTCCCAGGCCATCAAGGTGGTGAATGCCGT | |
| | GATCCGGAAGCACGGCTCTCCCGAGAGCATCCACATCGAGCTGGCCCGCGAGC | |
| | TGTCTAAGAACCTGGACGAGCGCAGGAAGATCGAGAAGGCCCAGAAGGAGAAT | |
| | CAGAAGAACAATGAGCAGATCAAGGACGAGATCAGGGAGATCCTGGGATCCGC | |
| | CCACGTGACCGGAAGAGATATCGTGAAGTACAAGCTGTTTAAGCAGCAGCAGG | |
| | AGTTCTGTATGTATTCTGGCGAGAAGCTGGACGTGACACGGCTGTTTGAGCCA | |
| | GGCTACGCCGAGGTGGATCACATCATCCCCTACGGCATCTCCTTCGACGATTC | |
| | TTATGACAACAAGGTGCTGGTGAAAACCGAGCAGAACCGGCAGAAGGGCAATC | |
| | GCACACCACTGGAGTATCTGCGGGATAAGCCCGAGCAGAAGGCCAAGTTTATC | |
| | GCCCTGGTGGAGAGCATCCCCCTGTCCCAGAAGAAGAAGAACCACCTGCTGAT | |
| | GGACAAGCGCGCCATCGATCTGGAGCAGGAGGGCTTTCGCGAGAGGAACCTGA | |
| | GCGACACCAGGTACATCACAAGAGCCCTGATGAATCACATCCAGGCCTGGCTG | |
| | CTGTTCGATGAGACCGCCTCCACACGGTCTAAGAGGGTGGTGTGCGTGAACGG | |
| | AGCAGTGACCGCCTATATGAGAGCCCGGTGGGGCCTGACAAAGGACAGGAATG | |
| | CAGGCGATAAGCACCACGCAGCAGACGCAGTGGTGGTGGCCTGTATCGGCGAT | |
| | AGCCTGATCCAGAGGGTGACCAAGTACGACAAGTTCAAGAGAAACGCCCTGGC | |
| | CGATCGCAATAGATACGTGCAGCAGGTGAGCAAGTCCGAGGGCATCACACAGT | |
| | ATGTGGACAAGGAGACCGGCGAGGTGTTCACATGGGAGTCCTTTGATGAGAGG | |
| | AAGTTCCTGCCTAACGAGCCACTGGAGCCATGGCCCTTCTTTAGGGACGAGCT | |
| | GCTGGCAAGGCTGTCTGACGATCCTAGCAAGAATATCAGAGCCATCGGCCTGC | |
| | TGACCTACTCTGAGACAGAGCAGATCGATCCCATCTTTGTGAGCCGGATGCCT | |
| | ACCAGGAAGGTGACAGGAGCAGCACACAAGGAGACCATCAGGTCTCCAAGAAT | |
| | CGTGAAGGTGGACGATAACAAGGGCACAGAGATCCAGGTGGTGGTGAGCAAGG | |
| | TGGCCCTGACCGAGCTGAAGCTGACAAAGGACGGCGAGATCAAGGATTACTTC | |
| | AGGCCCGAGGACGATCCTAGACTGTATAATACCCTGAGAGAGCGGCTGGTGCA | |
| | GTTTGGAGGCGACGCAAAGGCAGCCTTCAAGGAGCCCGTGTACAAGATCTCCA | |
| | AGGATGGCTCTGTGAGGACCCCAGTGAGAAAGGTGAAGATCCAGGAGAAGCTG | |
| | ACACTGGGAGTGCCAGTGCACGGAGGAAGGGGAATCGCCGAGAACGGCGGCAT | |
| | GGTGCGCATCGACGTGTTTGCCAAGGATGGCAAGTACTATTTCGTGCCCATCT | |
| | ACGTGGCCGACGTGCTGAAGAGGGAGCTGCCAAACAGACTGGCAACCGCACAC | |
| | AAGCCTTATTCTGAGTGGAGGGTGGTGGACGATTCTTACCAGTTCAAGTTTAG | |
| | CCTGTATCCTAATGATGCCGTGATGATCAAGCCAAGCAGAGAGGTGGACATCA | |
| | CCTACAAGGATAGGAAGGAGCCAGTGGGACTGAGGCTGATGTATTTCGCAGGA | |
| | GCAGACATCTCTGGAGCCACCCTGACACTGCGGACACACGATAACAGCGGAGA | |
| | GCTGAAGGGACTGGGAATCCAGGGCCTGGAGGTGTTTGAGAAGTACGTGGTGG | |
| | GACCTCTGGGCGACACCCACCCAGTGTATAAGGAGAGACGGATGCCTTTCCGG | |
| | GTGGAGCGCAAGATGAATGCTAGCAAAAGGCCGGCGGCCACGAAAAAGGCCGG | |
| | CCAGGCAAAAAAGAAAAAGtaa | |
| | (SEQ ID NO: 89) | |

§Spacer and complementary target strand sequences are shown in red and in italics. Mismatched bases on non-target strand are marked in green and in lowercase letters. PAMs on the non-target strand for AtCas9/AhCas9 are highlighted in yellow and is in bold (which are the last four nucleotides of the PAMs). The underlined parts are PAMs for SpCas9.
※The duplex consisted of this sequence and its complementary strand was cloned into pCE2 plasmid using TA/Blunt-Zero Cloning Kit (Vazyme)
□The duplex consisted of this sequence and its complementary strand was cloned into pCR plasmid using Zero
Blunt ™ TOPO ™ PCR Cloning Kit (Invitrogen)

Additional sequences involved in above examples:

tracrRNA-At/Ah 6-96:
(SEQ ID NO: 70)
ACCACAUCAAGGCUUGCGAGGUUGCUAUGAUAAGGCAACAGGCCGCAAA
GCACUGACCCGCAUUCCAAUGAAUGCGGGUCAUCUACUUUUU
tracrRNA-At/Ah 15-96:
(SEQ ID NO: 71)
AGGCUUGCGAGGUUGCUAUGAUAAGGCAACAGGCCGCAAAGCACUGACC
CGCAUUCCAAUGAAUGCGGGUCAUCUACUUUUU
tracrRNA-At/Ah 27-96:
(SEQ ID NO: 72)
UUGCUAUGAUAAGGCAACAGGCCGCAAAGCACUGACCCGCAUUCCAAUG
AAUGCGGGUCAUCUACUUUUU
crRNA-At/Ah duplex-forming sequence (21a crRNA-
At/Ah 31-66):
(SEQ ID NO: 73)
GUCAUAGUUCCCUCACAAGCCUCGAUGUGGUAUGAU
truncated crRNA-At/Ah duplex-forming sequence
(21a crRNA-At/Ah 31-52):
(SEQ ID NO: 74)
GUCAUAGUUCCCUCACAAGCCU

### Example 10. PI Domain Protein Variants

Given that AtCas9 has a very relaxed PAM (CNNN and RNNA, R=A, G, N=A, T, C, G) towards linear DNA, we then determined whether it is possible to engineer AtCas9 Pam-interacting domain to generate a Pamless variant that can bypass the PAM restriction regardless of DNA topology.

Methods: we focused on the Pam-interacting domain, generated 13 different protein variants and tested their function on WT PAM: CAGA and MUT PAM: TAGA using linear and Negative supercoiled DNA substrates.

Results: From the initial screen, we found two protein variants D1089A and T1096A both showed cleavage against MUT PAM regardless of DNA topology.

TABLE 2
listed protein variants that are used in the experiments

| | PI domain Amino | | |
|---|---|---|---|
| | acid mutants | Description | Function |
| AT-PI-m3 | S1064 R1065 L1080 | Different amino acids align | Null allele |
| | L1082 A1086 G1087 | with AhCas9 PI domain. | |
| | D1089 S1091 G1092 | | |
| | T1094 L1095 T1096 | | |
| | K1107 G1123A | | |
| AT-PI-m4 | D1089A | Conserved sites align with | PAMless cleavage: |
| | | NmeCas9. | NNNN (pam) |
| AT-PI-m5 | D1089A S1091A G1092A | Conserved sites align with | Enhanced cleavage |
| | | NmeCas9. | |
| AT-PI-m6 | D1089A S1091A G1092A | Conserved sites align with | Enhanced cleavage |
| | T1094AL1095A T1096A | NmeCas9 and CjCas9. | |
| AT-PI-m7 | R1065A T1094A T1096A | Polar amino acids and basic | Enhanced cleavage |
| | | amino acids. | |
| AT-PI-m8 | R1065A T1094A T1096A | m5+m7 | Enhanced cleavage |
| | D1089A S1091A G1092A | | |
| AT-PI-m9 | S1091A | Conserved sites align with | Enhanced cleavage |
| | | NmeCas9. | |
| AT-PI-m10 | G1092A | Conserved sites align with | Enhanced cleavage |
| | | NmeCas9. | |
| AT-PI-m11 | R1065A | Polar amino acids and basic | Enhanced cleavage |
| | | amino acids. | |
| AT-PI-m12 | T1094A | Polar amino acids and basic | Enhanced cleavage |
| | | amino acids. | |
| AT-PI-m13 | T1096A | Polar amino acids and basic | PAMless cleavage |
| | | amino acids. | |
| AT-PI-m14 | D1089E | Conserved sites align with | PAMless cleavage |
| | | NmeCas9. | |
| AT-PI-m15 | D1089A T1096A | m4+m13 | PAMless cleavage |
| V13 | 915-933 | Align with the regions with | Enhanced cleavage |
| | QLKLKDLEKMVNRERE | large conformational changes | |
| | | after NmeCas9 binds to DNA | |
| V38 | E1066K | Align with the regions with | Enhanced cleavage |
| | | large conformational changes | |
| | | after NmeCas9 binds to DNA | |
| V41 | D1068K | Align with the regions with | Enhanced cleavage |
| | | large conformational changes | |
| | | after NmeCas9 binds to DNA | |
| V4 | E530A S531R L536T | Align with the regions with | Enhanced cleavage |
| | | large conformational changes | |
| | | after NmeCas9 binds to DNA | |
| V58 | 915-933 | V7 + V13 | Enhanced cleavage |
| | QLKLKDLEKMVNRERE | | |
| | L602I D603N V604L | | |
| | T605G | | |
| V40 | D1068R | Align with the regions with | Enhanced cleavage |
| | | large conformational changes | |
| | | after NmeCas9 binds to DNA | |
| V7 | L602I D603N V604L | Align with the regions with | Enhanced cleavage |
| | T605G | large conformational changes | |
| | | after NmeCas9 binds to DNA | |
| V9 | 680-699 | Align with the regions with | Enhanced cleavage |
| | LLQKFDEDGFKERNLN | large conformational changes | |
| | | after NmeCas9 binds to DNA | |
| V59 | 915-933 | V13 + V4 + V7 | Enhanced cleavage |
| | QLKLKDLEKMVNRERE | | |
| | L602I D603N V604L | | |
| | T605G E530A S531R | | |
| | L536T | | |

The present disclosure is not to be limited in scope by the specific embodiments described which are intended as single illustrations of individual aspects of the disclosure, and any compositions or methods which are functionally equivalent are within the scope of this disclosure. It will be apparent to those skilled in the art that various modifications and variations can be made in the methods and compositions of the present disclosure without departing from the spirit or scope of the disclosure. Thus, it is intended that the present disclosure cover the modifications and variations of this disclosure provided they come within the scope of the appended claims and their equivalents.

All publications and patent applications mentioned in this specification are herein incorporated by reference to the same extent as if each individual publication or patent application was specifically and individually indicated to be incorporated by reference.

1. A method for editing a target nucleic acid, comprising contacting the target nucleic acid with a CRISPR-Cas system comprising:
a Cas9 protein derived from Alicyclobacillus sp. or a functional variant thereof, wherein the functional variant has at least 70% sequence identity to the Cas9 protein derived from Alicyclobacillus sp., and
a guide RNA comprising a guide sequence that hybridizes to a target sequence in the target nucleic acid,
wherein the target sequence (a) is adjacent to a protospacer adjacent motif (PAM) comprising CNNN or RNNA, wherein R is A, or G, and each N is independently A, T, C, or G, or (b) has an underwound topology.

2. The method of claim 1, wherein the Cas9 protein is derived from Alicyclobacillus tengchongensis, Alicyclobacillus hesperidum, or Alicyclobacillus Sacchari.

3. The method of claim 2, wherein the Cas9 protein derived from Alicyclobacillus sp. comprises the amino acid sequence of SEQ ID NO: 84 or SEQ ID NO:85.

4. The method of claim 1, wherein the target sequence is negatively supercoiled DNA, bulged double stranded DNA, or Z-DNA.

5. The method of claim 4, wherein the bulged DNA has one or more consecutive unpaired bases within positions 1-10 from 3′ of the complementary sequence of the target sequence.

6. The method of claim 1, wherein the target sequence having underwound topology does not include the PAM.

7. A mutant Cas9 protein, comprising (a) SEQ ID NO:84 with at least a mutation at a residue selected from the group consisting of E530, S531, L536, L602, D603, V604, T605, R1065, E1066, D1068, D1089, S1091, G1092, T1094, L1095, and T1096, or (b) a sequence having at least 70% sequence identity to SEQ ID NO:84 while retaining the mutation of (a).

8. The mutant Cas9 protein of claim 7, wherein the mutation is selected from the group consisting of E530A, S531R, L536T, L602I, D603N, V604L, T605G, R1065A, E1066K, D1068K, D1068R, D1089A, D1089E, S1091A, G1092A, T1094A, L1095A, and T1096A.

9. The mutant Cas9 protein of claim 7, wherein the mutation is at D1089 or T1096.

10. The mutant Cas9 protein of claim 9, wherein the mutation is D1089A or T1096A, or the combination thereof.

11. A fusion protein comprising the mutant Cas9 protein of claim 7 and a nucleobase deaminase or a reverse transcriptase.

12. A method for editing a target nucleic acid, comprising contacting the target nucleic acid with a CRISPR-Cas system comprising:
a Cas protein with a corresponding protospacer adjacent motif (PAM) required for targeting a linear double stranded DNA, and
a guide RNA comprising a guide sequence that hybridizes to a target sequence in the target nucleic acid, adjacent to a target PAM sequence,
wherein the target sequence has an underwound topology and the target PAM sequence is not the corresponding PAM of the Cas protein.

13. The method of claim 12, wherein the Cas protein is SpCas9 and the corresponding PAM is NGG, wherein N is A, T, C or G.

14. The method of claim 13, wherein the target PAM sequence is NAG or NGA.

15. The method of claim 12, wherein the Cas protein is FnCas9 and the corresponding PAM is NGG, wherein N is A, T, C or G.

16. The method of claim 15, wherein the target PAM sequence is NGA.

17. The method of claim 12, wherein the Cas protein is SaCas9 and the corresponding PAM is NNGRRT, wherein each N is independently A, G, C or T, and each R is independently A or G.

18. (canceled)

19. The method of claim 12, wherein the Cas protein is NmeCas9 and the corresponding PAM is NNNNGATT, wherein each N is independently A, G, C or T.

20. (canceled)

21. The method of claim 12, wherein the Cas protein is AsCas12a and the corresponding PAM is TTTV, wherein V is A, C or G.

22. (canceled)

23. The method of claim 12, wherein the Cas protein is AtCas9 and the corresponding PAM is CNNN or RNNA, wherein each N is independently A, T, C or G, and R is A or G.

24-35. (canceled)


SEQUENCE LISTING (89 sequences)

SEQ ID NO: 1 - RNA, 96 nt, Artificial Sequence
  Synthetic
aucauaccacaucaaggcuugcgagguugcuaugauaaggcaacaggccgcaaagcacugacccgcauuccaaugaaugcgggucaucuacuuuuu

SEQ ID NO: 2 - RNA, 66 nt, Artificial Sequence
  Synthetic
aaaugccuucgauuuuaccccaacgaauaagucauaguucccucacaagccucgaugugguaugau

SEQ ID NO: 3 - RNA, 88 nt, Artificial Sequence
  Synthetic
aaaugagaaccguugcuacaauaaggccgucugaaaagaugugccgcaacgcucugccccuuaaagcuucugcuuuaaggggcuuuuu

SEQ ID NO: 4 - RNA, 48 nt, Artificial Sequence
  Synthetic
cuucgauuuuaccccaacgaauaaguuguagcucccuuucucauuucg

SEQ ID NO: 5 - RNA, 157 nt, Artificial Sequence
  Synthetic
uucacugauaaaguggagaaccgcuucaccaaaagcugucccuuaggggauuagaacuugagugaaggugggcugcuugcaucagccuaaugucgagaagugcuuucuucggaaaguaacccucgaaacaaauucauuuuuccucuccaauucugca

SEQ ID NO: 6 - RNA, 65 nt, Artificial Sequence
  Synthetic
ugcagaacccgaauagacgaaugaaggaaugcaacaaaugccuucgauuuuaccccaacgaauaa

SEQ ID NO: 7 - RNA, 102 nt, Artificial Sequence
  Synthetic
gauuuuaccccaacgaauaaguuuuagagcuagaaauagcaaguuaaaauaaggcuaguccguuaucaacuugaaaaaguggcaccgagucggugcuuuuuu

SEQ ID NO: 8 - RNA, 102 nt, Artificial Sequence
  Synthetic
uuaccccaacgaauaaguacguuuuagagcuagaaauagcaaguuaaaauaaggcuaguccguuaucaacuugaaaaaguggcaccgagucggugcuuuuuu

SEQ ID NO: 9 - RNA, 166 nt, Artificial Sequence
  Synthetic
aaaugccuucgauuuuaccccaacgaauaagucauaguucccucacaagccucgaugugguaugaugaaaaucauaccacaucaaggcuugcgagguugcuaugauaaggcaacaggccgcaaagcacugacccgcauuccaaugaaugcgggucaucuacuuuuu

SEQ ID NO: 10 - RNA, 138 nt, Artificial Sequence
  Synthetic
aaaugccuucgauuuuaccccaacgaauaagucauaguucccucacaagccugaaaaggcuugcgagguugcuaugauaaggcaacaggccgcaaagcacugacccgcauuccaaugaaugcgggucaucuacuuuuu

SEQ ID NO: 11 - RNA, 161 nt, Artificial Sequence
  Synthetic
gaucgcacauacguacacauugaccgucauaguucccucacaagccucgaugugguaugaugaaaaucauaccacaucaaggcuugcgagguugcuaugauaaggcaacaggccgcaaagcacugacccgcauuccaaugaaugcgggucaucuacuuuuu

SEQ ID NO: 12 - RNA, 161 nt, Artificial Sequence
  Synthetic
acauacguacacauugaccuauuuagucauaguucccucacaagccucgaugugguaugaugaaaaucauaccacaucaaggcuugcgagguugcuaugauaaggcaacaggccgcaaagcacugacccgcauuccaaugaaugcgggucaucuacuuuuu

SEQ ID NO: 13 - RNA, 161 nt, Artificial Sequence
  Synthetic
gaagauccuuugaucuuuucuacgggucauaguucccucacaagccucgaugugguaugaugaaaaucauaccacaucaaggcuugcgagguugcuaugauaaggcaacaggccgcaaagcacugacccgcauuccaaugaaugcgggucaucuacuuuuu

SEQ ID NO: 14 - RNA, 161 nt, Artificial Sequence
  Synthetic
gccagggcacgggcagcuugccggugucauaguucccucacaagccucgaugugguaugaugaaaaucauaccacaucaaggcuugcgagguugcuaugauaaggcaacaggccgcaaagcacugacccgcauuccaaugaaugcgggucaucuacuuuuu

SEQ ID NO: 15 - RNA, 161 nt, Artificial Sequence
  Synthetic
agggcgaggagcuguucaccggggugucauaguucccucacaagccucgaugugguaugaugaaaaucauaccacaucaaggcuugcgagguugcuaugauaaggcaacaggccgcaaagcacugacccgcauuccaaugaaugcgggucaucuacuuuuu

SEQ ID NO: 16 - RNA, 66 nt, Artificial Sequence
  Synthetic
caugagacaauaacccugauaaaugcuucagucauaguucccucacaagccucgaugugguaugau

SEQ ID NO: 17 - RNA, 66 nt, Artificial Sequence
  Synthetic
ugaaugucagcuacugggcuaucuggacaagucauaguucccucacaagccucgaugugguaugau

SEQ ID NO: 18 - RNA, 66 nt, Artificial Sequence
  Synthetic
acggcucucucuuuuauagguguaaaccuugucauaguucccucacaagccucgaugugguaugau

SEQ ID NO: 19 - RNA, 66 nt, Artificial Sequence
  Synthetic
accuguagacagcaugaaguuaggauuguggucauaguucccucacaagccucgaugugguaugau

SEQ ID NO: 20 - RNA, 66 nt, Artificial Sequence
  Synthetic
cgccauuaaccugauguucuggggaauauagucauaguucccucacaagccucgaugugguaugau

SEQ ID NO: 21 - RNA, 66 nt, Artificial Sequence
  Synthetic
uuauucguugggguaaaaucgaaggcauuugucauaguucccucacaagccucgaugugguaugau

SEQ ID NO: 22 - RNA, 66 nt, Artificial Sequence
  Synthetic
gauguugagcuccguuuggggguauggugagucauaguucccucacaagccucgaugugguaugau

SEQ ID NO: 23 - DNA, 38 bp, Artificial Sequence
  Synthetic
aaatgccttcgattttaccccaacgaataagtaccata

SEQ ID NO: 24 - DNA, 38 bp, Artificial Sequence
  Synthetic
aaatgccttcgattttaccccaacgaataagtacgata

SEQ ID NO: 25 - DNA, 38 bp, Artificial Sequence
  Synthetic
aaatgccttcgattttaccccaacgaataagtcatata

SEQ ID NO: 26 - DNA, 38 bp, Artificial Sequence
  Synthetic
aaatgccttcgattttaccccaacgaataagtaccggt

SEQ ID NO: 27 - DNA, 38 bp, Artificial Sequence
  Synthetic
aaatgccttcgattttaccccaacgaataagtactagt

SEQ ID NO: 28 - DNA, 38 bp, Artificial Sequence
  Synthetic
aaatgccttcgattttaccccaacgaataagtaccagg

SEQ ID NO: 29 - DNA, 38 bp, Artificial Sequence
  Synthetic
aaatgccttcgattttaccccaacgaataagtacaaga

SEQ ID NO: 30 - DNA, 38 bp, Artificial Sequence
  Synthetic
gttgtggctgttgtagttgtactccagcttgtgcccca

SEQ ID NO: 31 - DNA, 60 bp, Artificial Sequence
  Synthetic
ccttatccgaccaacatatcataacggagtgatcgcattggagctctggtttgtggggca

SEQ ID NO: 32 - DNA, 60 bp, Artificial Sequence
  Synthetic
taggcctgataagcgcagcgtatcaggcaatttttataatccgggaagccctgggccaac

SEQ ID NO: 33 - DNA, 50 bp, Artificial Sequence
  Synthetic
caggtccatatttatatgacttattcgttggggtaaaatcgaaggcattt

SEQ ID NO: 34 - DNA, 50 bp, Artificial Sequence
  Synthetic
aaatgccttcgattttaccccaacgaataagtcatataaatatggacctg

SEQ ID NO: 35 - DNA, 50 bp, Artificial Sequence
  Synthetic
aaatgccttcgattttaccccaacgaatttgtcatataaatatggacctg

SEQ ID NO: 36 - DNA, 50 bp, Artificial Sequence
  Synthetic
aaatgccttcgattttaccccaacgataaagtcatataaatatggacctg

SEQ ID NO: 37 - DNA, 50 bp, Artificial Sequence
  Synthetic
aaatgccttcgattttaccccaacctataagtcatataaatatggacctg

SEQ ID NO: 38 - DNA, 50 bp, Artificial Sequence
  Synthetic
aaatgccttcctttttaccccaacgaataagtcatataaatatggacctg

SEQ ID NO: 39 - DNA, 50 bp, Artificial Sequence
  Synthetic
aaatgcctaggattttaccccaacgaataagtcatataaatatggacctg

SEQ ID NO: 40 - DNA, 50 bp, Artificial Sequence
  Synthetic
aaatgggatcgattttaccccaacgaataagtcatataaatatggacctg

SEQ ID NO: 41 - DNA, 50 bp, Artificial Sequence
  Synthetic
aaatgccttcgattttaccccaacgaataagtcatataaatatccacctg

SEQ ID NO: 42 - DNA, 50 bp, Artificial Sequence
  Synthetic
caggtccatattttagcctgttattcgttggggtaaaatcgaaggcattt

SEQ ID NO: 43 - DNA, 50 bp, Artificial Sequence
  Synthetic
aaatgccttcgattttaccccaacgaataacaggctaaaatatggacctg

SEQ ID NO: 44 - DNA, 50 bp, Artificial Sequence
  Synthetic
aaatgccttcgattttaccccaacgaatttcaggctaaaatatggacctg

SEQ ID NO: 45 - DNA, 120 bp, Artificial Sequence
  Synthetic
aggtttaaacgaattcgcccttggtccatattttagcctgtatatgacttattcgttggggtaaaatcgaaggcatttgtgcaccaatttctgatgaagggcgaattcgcggccgctaaa

SEQ ID NO: 46 - DNA, 120 bp, Artificial Sequence
  Synthetic
tttagcggccgcgaattcgcccttcatcagaaattggtgcacaaatgccttcgattttaccccaacgaataagtcatatacaggctaaaatatggaccaagggcgaattcgtttaaacct

SEQ ID NO: 47 - DNA, 120 bp, Artificial Sequence
  Synthetic
tttagcggccgcgaattcgcccttcatcagaaattggtgcacaaatgccttcgattttaccccaacgaatttgtcatatacaggctaaaatatggaccaagggcgaattcgtttaaacct

SEQ ID NO: 48 - DNA, 120 bp, Artificial Sequence
  Synthetic
tttagcggccgcgaattcgcccttcatcagaaattggtgcacaaatgccttcgattttaccccaacgataaagtcatatacaggctaaaatatggaccaagggcgaattcgtttaaacct

SEQ ID NO: 49 - DNA, 120 bp, Artificial Sequence
  Synthetic
tttagcggccgcgaattcgcccttcatcagaaattggtgcacaaatgccttcgattttaccccaacctataagtcatatacaggctaaaatatggaccaagggcgaattcgtttaaacct

SEQ ID NO: 50 - DNA, 120 bp, Artificial Sequence
  Synthetic
tttagcggccgcgaattcgcccttcatcagaaattggtgcacaaatgccttcctttttaccccaacgaataagtcatatacaggctaaaatatggaccaagggcgaattcgtttaaacct

SEQ ID NO: 51 - DNA, 120 bp, Artificial Sequence
  Synthetic
tttagcggccgcgaattcgcccttcatcagaaattggtgcacaaatgcctaggattttaccccaacgaataagtcatatacaggctaaaatatggaccaagggcgaattcgtttaaacct

SEQ ID NO: 52 - DNA, 120 bp, Artificial Sequence
  Synthetic
tttagcggccgcgaattcgcccttcatcagaaattggtgcacaaatgggatcgattttaccccaacgaataagtcatatacaggctaaaatatggaccaagggcgaattcgtttaaacct

SEQ ID NO: 53 - DNA, 120 bp, Artificial Sequence
  Synthetic
tttagcggccgcgaattcgcccttcatcagaaattggtgcacaaatgccttcgattttaccccaacgaataagtcatatacaggctaaaatatggaggaagggcgaattcgtttaaacct

SEQ ID NO: 54 - DNA, 89 bp, Artificial Sequence
  Synthetic
gatcgagaactttcgaacccttataccatgaacgatcgccatttcgaatgtgtgtacgcgcgtaaataggtcaatgtgtacgtatgtgc

SEQ ID NO: 55 - DNA, 89 bp, Artificial Sequence
  Synthetic
gcacatacgtacacattgacctatttacgcgcgtacacacattcgaaatggcgatcgttcatggtataagggttcgaaagttctcgatc

SEQ ID NO: 56 - DNA, 33 bp, Artificial Sequence
  Synthetic
gatcgcacatacgtacacattgacctatttacg

SEQ ID NO: 57 - DNA, 33 bp, Artificial Sequence
  Synthetic
acatacgtacacattgacctatttacgcgcgta

SEQ ID NO: 58 - DNA, 33 bp, Artificial Sequence
  Synthetic
gaagatcctttgatcttttctacggggtcctaa

SEQ ID NO: 59 - DNA, 33 bp, Artificial Sequence
  Synthetic
gaagatcctttgatcttttctacggggtctgac

SEQ ID NO: 60 - DNA, 38 bp, Artificial Sequence
  Synthetic
aaatgccttcgattttaccccaacgaataacaggctaa

SEQ ID NO: 61 - DNA, 21 bp, Artificial Sequence
  Synthetic
ccaaagaagaagcggaaggtc

SEQ ID NO: 62 - DNA, 27 bp, Artificial Sequence
  Synthetic
ccagcagccaagagggtgaagctggat

SEQ ID NO: 63 - DNA, 48 bp, Artificial Sequence
  Synthetic
aaaaggccggcggccacgaaaaaggccggccaggcaaaaaagaaaaag

SEQ ID NO: 64 - DNA, 38 bp, Artificial Sequence
  Synthetic
catgagacaataaccctgataaatgcttcaataatagc

SEQ ID NO: 65 - DNA, 38 bp, Artificial Sequence
  Synthetic
tgaatgtcagctactgggctatctggacaagggaaaac

SEQ ID NO: 66 - DNA, 38 bp, Artificial Sequence
  Synthetic
acggctctctcttttataggtgtaaaccttaaactgcc

SEQ ID NO: 67 - DNA, 38 bp, Artificial Sequence
  Synthetic
acctgtagacagcatgaagttaggattgtgaaagagtt

SEQ ID NO: 68 - DNA, 38 bp, Artificial Sequence
  Synthetic
cgccattaacctgatgttctggggaatataaatgtcag

SEQ ID NO: 69 - DNA, 38 bp, Artificial Sequence
  Synthetic
gatgttgagctccgtttgggggtatggtgagtgtctag

SEQ ID NO: 70 - RNA, 91 nt, Artificial Sequence
  Synthetic
accacaucaaggcuugcgagguugcuaugauaaggcaacaggccgcaaagcacugacccgcauuccaaugaaugcgggucaucuacuuuuu

SEQ ID NO: 71 - RNA, 82 nt, Artificial Sequence
  Synthetic
aggcuugcgagguugcuaugauaaggcaacaggccgcaaagcacugacccgcauuccaaugaaugcgggucaucuacuuuuu

SEQ ID NO: 72 - RNA, 70 nt, Artificial Sequence
  Synthetic
uugcuaugauaaggcaacaggccgcaaagcacugacccgcauuccaaugaaugcgggucaucuacuuuuu

SEQ ID NO: 73 - RNA, 36 nt, Artificial Sequence
  Synthetic
gucauaguucccucacaagccucgaugugguaugau

SEQ ID NO: 74 - RNA, 22 nt, Artificial Sequence
  Synthetic
gucauaguucccucacaagccu

SEQ ID NO: 75 - RNA, 66 nt, Artificial Sequence
  Synthetic
ucacaggugccucuuagcugcuaagccugggucauaguucccucacaagccucgaugugguaugau

SEQ ID NO: 76 - RNA, 66 nt, Artificial Sequence; identical to SEQ ID NO: 22
  Synthetic

SEQ ID NO: 77 - skipped

SEQ ID NO: 78 - DNA, 1500 bp, Artificial Sequence
  Synthetic
aggctggagtatagtgatttgatcatggctgactgcaggcttgaacttttaggctcaagtgattctcccacctcagcttccagagtagctgggactacaggcatgcaccaccacacctggctaatatttttaattttttgtagagacagggtctcgctatgttgcccaggctggtctcgaactcctggcctcaagcagtctttctgccacagcctcccaaagtgctgagattataggtgtgagccattgtacctggcttggagtaatgtttttgagcaaagcaaatgtacaggacatgcacagagaggcacagggagcacagagtccctggatgaatgctctggctggtggcagtattgtcacagggcaggctacccaaaggtgagcagaacacatgaggctagacaggtacaacttgctcatgaagagcctcgaatgccaagcttaggagtttgaactttattggaacactgacactaatagttaatgtttactgagtgcttactatgtgctaagagctatgcatgaatcacctcattctaattctcttaactctccacagcaggtcttattatttccagattgctgatgaaaaactgagtcacagagaggtaaaaaaaaaaaaacaaaaacaccctgggtccagagttgtgtgaaaaaattttaacaggttagagttgtgttgtagaaatctggtagccagccacgtgtaagacgaattgtagaaaggagagatcacaaatggctatcataagagaatcactgggaaaaggtaataagaacccaaattagaggcaaggagaacagaaagaaatgaaagaatgcaaagactgactggatgtgcacagcagtgaccgagagaaaggaggtatcacaggtgcctcttagctgctaagcctgggtgtcctgcagaaaggggcagctattatcagaagagagaagccagagagaggcacagatttggtggggatgatgttgagctccgtttgggggtatggtgagtgtctagggggcctgtaggaacccctccagaaaaattctcacaagcatttgaaaatcagtgacttgatctggagaaaaatatagggctggcattacaaacctgccggggtctgcattacagtattggttgaaagtgtggaaattgataatgatagatggttggtagatacatagatagatagatagatagatagatagatagatagatagatagatagatagatagaataaatagcatgatacatggaatgagggaagggctaaggataaagatgccttcatggatgcccacagttaggagaagaaagtagagcccatgaaggaacaactcccagagtttgcagatccaggcaagtatcttgttcagaaaataaagggaagggagtttccaaaagaaaagggggttgaaggtactcagatgctgaggagcctataaacaattgtgactaaaaaagactcaaatttggaaataaggtgtcctaatgaacatttcagagg

SEQ ID NO: 79 - DNA, 1612 bp, Artificial Sequence
  Synthetic
cttccagggggttggttgtttatttcttccaaatgggtcataaaaggatgataaagctgcaaacccaaagaaactaattattcactacttgaaaggatgaagtgaattttgagctttatgaatgcaaactaataataagtatcgtggatagtaattatgcttacaattttaattagtgaatgctgttgtaaattataaggctgtgtctacattagaaacaaaaaacacggaactctaaacctgcaacaaacctgagtgtacatagactgagattctaagggacttttaagatgaaaaattcacatggtttcagataaaactcacacaggtcatcctgaaagtcactgttggtcgatggtcattagcactatagtttgtaacataataagaagaggtgggagtaatgtttctcttctggtaaaaaacattggcaattacatatgccaggaaacccgactgatttgagttgtaatgcacagatcgctagcgatgcagaaaaaaagtcaaacaatgccatgtgaagtgtactctctagacccaattagatcaatctttcccaaattacttagtacatgattggaaagagagcagctgtattttcaatccatcatcccatccacctatctcaaatttgcctgggttaattcttttgacccaccagttatatcaagtaacatatgtttttccctcaattgatcaactaacaaaatcgatgagaatttttatagtcacatagttatggagacaaattctcagatgaacatactttgagcaggggtgggattaggaggtacggaggggcaagggttaatgctgacagctgttaccaaagcctcttttcaagtccccgctttactgaagtttgttctatcttacatgcatcacacatcagaaattggtgcacaaatgccttcgattttaccccaacgaataacaggctaaaatatggacctgtagacagcatgaagttaggattgtgaaagagttatcttggattcatattttgacactcagaaaccttgcaaatgcttgatctcctctcttctaacaaaaggtctcagcagtagaccagcctcgggatctcaaaggctgagtggttggtgctggctcagccctgtccccaccggccactgccatctcgacattgccatctgacatcagagctacaggcgtgcctccctccacacagcacagattcattttgacaaactagtttggggtggcttggacagtgcaggaacagtagtttctggtgagtgatatcattagttcaggaggccagggtgcagcagcagcagcagccccccatttccatcccagctgcccaccctatacccacacacatacaccccgttacttggcccacaatcttccctcctaacatcttcaacttagtctgtacacaatttccaagtaataactgtgtgctcaagtgtgacctctcattatcacacctgttccacttattcattcactctgcaaatactatcgtctgttttttgccaaacatcatgtatcaaaaatgtgacaaaataaaaagcctttttataagtctcaagtatccacaaaataacactacaattcttgctcttccttcagacaacctcttgca

SEQ ID NO: 80 - DNA, 50 bp, Artificial Sequence; identical to SEQ ID NO: 42
  Synthetic

SEQ ID NO: 81 - RNA, 50 nt, Artificial Sequence
  Synthetic
cagguccauauuuuagccuguuauucguugggguaaaaucgaaggcauuu

SEQ ID NO: 82 - DNA, 3441 bp, Artificial Sequence
  Synthetic
atggcatatcgactgggattggatattggcattacatccgtagggtgggccgttgtggcacttgagaaggatgaaagcggattaaaacccgttcgcattcaagatcttggtgttcgcatttttgataaggctgaagacagtaagacgggggcatctttggctcttccccgccgtgaggcgcgcagtgcacggcgtcgcacgcgccgtcgtcgccatcggctttggcgcgtaaagcgtttgttggaacagcacgggattctttcgatggaacaaattgaagcgctttatgcgcaacgaacgtcatctcctgatgtgtatgctctgcgtgtggctgggctcgatagatgtctcatcgcagaggaaatcgcgagagtgcttattcatattgcgcatcgtcgcggttttcagtcaaataggaaatctgaaatcaaagattcagacgccggaaagttgctgaaggctgtgcaggaaaatgaaaatttaatgcaatcgaagggataccgaacggttgcggaaatgcttgtttctgaggcaacgaaaaccgatgctgagggaaaactcgttcatggtaagaaacacggttatgtatcgaatgtgagaaacaaggcaggcgagtatcgtcatacggtctcgcggcaagccattgtggacgaagttcggaagattttcgcagcccaacgagcgctgggtaatgatgtgatgagtgaagagttggaggattcatatctgaagattctttgctctcaacgcaacttcgatgatggcccaggtggagacagtccttatgggcatggttctgtctcgcctgatggcgtaaggcaaagtatttatgaacgaatggtaggctcgtgtacgttcgagacaggtgaaaagcgggcgccgcgcagttcgtattcgttcgagcggttccagctgcttaccaaggtcgtgaatcttcgcatctatcgacaacaggaggatggaggaagatatccgtgtgagctcacacagacagaacgagcacgcgtgattgattgtgcgtatgagcaaacgaaaattacgtatgggaaactgcggaagctgctagacatgaaagacacggaaagcttcgcgggacttacgtatggcttgaatcgtagccgtaataaaacggaggacacggtctttgtcgagatgaagttttatcacgaggtcagaaaggctctgcagcgtgctggggttttcattcaagatttgtccattgaaacgcttgatcaaatcggttggatcttgtctgtgtggaaaagcgatgacaaccgcagaaagaagctttcgactctcgggttgtccgacaatgtaattgaggaattactaccgttaaatggttcgaaatttggacacctttcgctgaaggccattcgaaaaatacttccgttcttagaggatggctacagctatgatgtagcctgcgagctggctgggtatcagtttcaagggaaaacagagtacgtaaaacagaggcttttgccacctttgggtgaaggagaagtgaccaatccggtggtgcggcgtgccctttcacaggccatcaaagttgtgaatgcggtcatccgcaagcatggttcgcccgaatctattcacatcgagcttgcccgggagctatccaagaatctggatgagcggcgtaaaattgagaaggctcagaaggaaaaccaaaaaaataacgagcagatcaaggacgagattcgtgagattcttggaagtgcgcatgttaccggacgagacattgtgaagtacaagctattcaagcagcagcaggaattctgcatgtattctggggaaaagttggacgtcacacgtctattcgagccaggttacgccgaagtggatcacatcatcccgtatgggatttcatttgacgatagctatgacaacaaggttcttgtcaagacggagcaaaatcgtcagaaggggaaccgaacgccattggagtatctgcgagataagccagagcagaaggcaaagtttatagcgttggtagaaagtatcccattgtcgcaaaagaagaagaaccatttgttaatggacaagcgcgcgatcgatttggaacaagaaggatttcgcgagagaaatctttcggacacgcggtacatcaccagggctttgatgaatcacattcaggcatggttgttatttgacgaaacggcttccacgcggtcaaagcgtgtggtttgtgtcaatggcgccgttacggcatatatgcgagcgcggtggggattgacgaaggatcggaatgccggtgataagcaccatgcggccgatgcagttgttgttgcgtgcatcggagattcgcttattcaacgcgtcacgaagtatgacaaatttaagcgcaatgcgctcgccgatcgcaaccggtatgtgcagcaggtcagcaaaagcgaaggcatcacgcaatatgtggataaagagacgggtgaggtgttcacgtgggaatcatttgacgaacgcaagtttctcccaaacgaacctttggagccctggccattctttcgagacgagctgttggcaagactcagcgacgatcctagcaaaaatatacgagctattggcctcttaacctattcggaaaccgagcaaattgatcctatctttgtctcgagaatgccgacaaggaaggtcacgggcgcggctcacaaggagacgattcgtagcccgcgtatcgtgaaagtcgatgataataagggcacagagattcaggtcgtggtttcaaaggtggctcttaccgaactcaagctcacaaaggatggggagattaaggattatttccgaccggaagatgatcctagattgtacaacactctgcgcgaaaggcttgttcaatttggcggcgatgcgaaggctgcttttaaagaacctgtttacaagatttcgaaggacggtagtgtgcgaacacccgttaggaaggtcaagattcaagaaaagttgacgctcggtgtacctgttcatggcggacgaggcatcgcagagaatgggggaatggtgagaattgatgtgtttgcaaaagacggaaagtattactttgtaccgatttatgtcgcagatgtactgaagcgggaacttccgaatcgcctagccactgcgcacaagccgtactccgagtggagagttgtggatgatagctatcaatttaaatttagcttatatccaaacgacgcggtgatgattaaacctagtagggaagtggatataacttataaggacaggaaggaacctgttggattacgactgatgtattttgcgggtgcggatatttcaggcgctactttaactctgcgtacgcacgacaattctggagaactgaagggtttgggtatacaaggtctagaggtgttcgaaaaatacgttgttggtccgttaggagacacgcatcctgtttataaggaacggcgcatgccatttcgtgtggaaaggaagatgaattga

SEQ ID NO: 83 - DNA, 3441 bp, Artificial Sequence
  Synthetic
atggcctacagactgggcctggacatcggcatcacctccgtgggatgggcagtggtggccctggagaaggacgagtctggcctgaagcccgtgcggatccaggatctgggcgtgcgcatctttgacaaggccgaggatagcaagaccggcgcctccctggccctgcctaggagagaggcacggagcgcccggcgcaggacaagacggcgcaggcacaggctgtggagggtgaagagactgctggagcagcacggcatcctgtccatggagcagatcgaggccctgtacgcacagcgcaccagctccccagacgtgtatgcactgagggtggcaggactggataggtgcctgatcgccgaggagatcgccagggtgctgatccacatcgcccacagacggggcttccagagcaacagaaagtccgagatcaaggacagcgatgccggcaagctgctgaaggccgtgcaggagaacgagaatctgatgcagtccaagggctaccggacagtggccgagatgctggtgtctgaggccaccaagacagacgccgagggcaagctggtgcacggcaagaagcacggctacgtgagcaacgtgcgcaataaggcaggagagtataggcacaccgtgtccaggcaggcaatcgtggatgaggtgaggaagatctttgcagcacagagagccctgggaaacgacgtgatgtccgaggagctggaggattcttacctgaagatcctgtgcagccagaggaatttcgacgatggaccaggaggcgactccccatacggacacggatccgtgtctcctgatggcgtgaggcagtccatctatgagagaatggtgggctcttgtaccttcgagacaggagagaagagggcaccacgctctagctattccttcgagcggtttcagctgctgaccaaggtggtgaacctgaggatctacagacagcaggaggacggcggccgctatccttgcgagctgacccagacagagcgcgccagagtgatcgattgtgcctacgagcagaccaagatcacatatggcaagctgagaaagctgctggacatgaaggataccgagagctttgccggcctgacatacggcctgaacagatcccggaataagaccgaggacacagtgttcgtggagatgaagttttatcacgaggtgaggaaggccctgcagagagccggcgtgttcatccaggacctgagcatcgagaccctggatcagatcggctggatcctgagcgtgtggaagagcgacgataaccgcaggaagaagctgtctacactgggcctgagcgacaacgtgatcgaggagctgctgcccctgaatggcagcaagtttggccacctgtccctgaaggccatcaggaagatcctgcctttcctggaggacggctactcctatgatgtggcctgcgagctggccggctaccagttccagggcaagaccgagtatgtgaagcagagactgctgccacctctgggagagggagaggtgacaaacccagtggtgagacgggccctgtcccaggccatcaaggtggtgaatgccgtgatccggaagcacggctctcccgagagcatccacatcgagctggcccgcgagctgtctaagaacctggacgagcgcaggaagatcgagaaggcccagaaggagaatcagaagaacaatgagcagatcaaggacgagatcagggagatcctgggatccgcccacgtgaccggaagagatatcgtgaagtacaagctgtttaagcagcagcaggagttctgtatgtattctggcgagaagctggacgtgacacggctgtttgagccaggctacgccgaggtggatcacatcatcccctacggcatctccttcgacgattcttatgacaacaaggtgctggtgaaaaccgagcagaaccggcagaagggcaatcgcacaccactggagtatctgcgggataagcccgagcagaaggccaagtttatcgccctggtggagagcatccccctgtcccagaagaagaagaaccacctgctgatggacaagcgcgccatcgatctggagcaggagggctttcgcgagaggaacctgagcgacaccaggtacatcacaagagccctgatgaatcacatccaggcctggctgctgttcgatgagaccgcctccacacggtctaagagggtggtgtgcgtgaacggagcagtgaccgcctatatgagagcccggtggggcctgacaaaggacaggaatgcaggcgataagcaccacgcagcagacgcagtggtggtggcctgtatcggcgatagcctgatccagagggtgaccaagtacgacaagttcaagagaaacgccctggccgatcgcaatagatacgtgcagcaggtgagcaagtccgagggcatcacacagtatgtggacaaggagaccggcgaggtgttcacatgggagtcctttgatgagaggaagttcctgcctaacgagccactggagccatggcccttctttagggacgagctgctggcaaggctgtctgacgatcctagcaagaatatcagagccatcggcctgctgacctactctgagacagagcagatcgatcccatctttgtgagccggatgcctaccaggaaggtgacaggagcagcacacaaggagaccatcaggtctccaagaatcgtgaaggtggacgataacaagggcacagagatccaggtggtggtgagcaaggtggccctgaccgagctgaagctgacaaaggacggcgagatcaaggattacttcaggcccgaggacgatcctagactgtataataccctgagagagcggctggtgcagtttggaggcgacgcaaaggcagccttcaaggagcccgtgtacaagatctccaaggatggctctgtgaggaccccagtgagaaaggtgaagatccaggagaagctgacactgggagtgccagtgcacggaggaaggggaatcgccgagaacggcggcatggtgcgcatcgacgtgtttgccaaggatggcaagtactatttcgtgcccatctacgtggccgacgtgctgaagagggagctgccaaacagactggcaaccgcacacaagccttattctgagtggagggtggtggacgattcttaccagttcaagtttagcctgtatcctaatgatgccgtgatgatcaagccaagcagagaggtggacatcacctacaaggataggaaggagccagtgggactgaggctgatgtatttcgcaggagcagacatctctggagccaccctgacactgcggacacacgataacagcggagagctgaagggactgggaatccagggcctggaggtgtttgagaagtacgtggtgggacctctgggcgacacccacccagtgtataaggagagacggatgcctttccgggtggagcgcaagatgaattga

SEQ ID NO: 84 - protein, 1146 aa, Artificial Sequence
  Synthetic
MAYRLGLDIGITSVGWAVVALEKDESGLKPVRIQDLGVRIFDKAEDSKTGASLALPRREARSARRRTRRRRHRLWRVKRLLEQHGILSMEQIEALYAQRTSSPDVYALRVAGLDRCLIAEEIARVLIHIAHRRGFQSNRKSEIKDSDAGKLLKAVQENENLMQSKGYRTVAEMLVSEATKTDAEGKLVHGKKHGYVSNVRNKAGEYRHTVSRQAIVDEVRKIFAAQRALGNDVMSEELEDSYLKILCSQRNFDDGPGGDSPYGHGSVSPDGVRQSIYERMVGSCTFETGEKRAPRSSYSFERFQLLTKVVNLRIYRQQEDGGRYPCELTQTERARVIDCAYEQTKITYGKLRKLLDMKDTESFAGLTYGLNRSRNKTEDTVFVEMKFYHEVRKALQRAGVFIQDLSIETLDQIGWILSVWKSDDNRRKKLSTLGLSDNVIEELLPLNGSKFGHLSLKAIRKILPFLEDGYSYDVACELAGYQFQGKTEYVKQRLLPPLGEGEVTNPVVRRALSQAIKVVNAVIRKHGSPESIHIELARELSKNLDERRKIEKAQKENQKNNEQIKDEIREILGSAHVTGRDIVKYKLFKQQQEFCMYSGEKLDVTRLFEPGYAEVDHIIPYGISFDDSYDNKVLVKTEQNRQKGNRTPLEYLRDKPEQKAKFIALVESIPLSQKKKNHLLMDKRAIDLEQEGFRERNLSDTRYITRALMNHIQAWLLFDETASTRSKRVVCVNGAVTAYMRARWGLTKDRNAGDKHHAADAVVVACIGDSLIQRVTKYDKFKRNALADRNRYVQQVSKSEGITQYVDKETGEVFTWESFDERKFLPNEPLEPWPFFRDELLARLSDDPSKNIRAIGLLTYSETEQIDPIFVSRMPTRKVTGAAHKETIRSPRIVKVDDNKGTEIQVVVSKVALTELKLTKDGEIKDYFRPEDDPRLYNTLRERLVQFGGDAKAAFKEPVYKISKDGSVRTPVRKVKIQEKLTLGVPVHGGRGIAENGGMVRIDVFAKDGKYYFVPIYVADVLKRELPNRLATAHKPYSEWRVVDDSYQFKFSLYPNDAVMIKPSREVDITYKDRKEPVGLRLMYFAGADISGATLTLRTHDNSGELKGLGIQGLEVFEKYVVGPLGDTHPVYKERRMPFRVERKMN

SEQ ID NO: 85 - protein, 1146 aa, Artificial Sequence
  Synthetic
MAYRLGLDIGITSVGWAVVALEKDESGLKPIRIQDLGVRIFDKAEDSKTGASLALPRREARSARRRTRRRRHRLWRVKRLLEQHGILSMEQIEALYAQRTSSPDVYALRVAGLDRCFTAEEIARVLIHIAHRRGFQSNRKSEIKDSDAGKLLKAVQENENLMQSKGYRTVAEMLVSEATKTDAEGKLVHGKKHGYVSNVRNKEGEYRHTVSRQAIVDEVRKIFAAQRALGNDVMSEELEDSYLKILRSQRNFDDGPGGDSPYGHGSVSPDGVRQSIYERMVGSCTFETGEKRAPRSSYSFERFQLLTKVVNLRIYRQQEDGGRYPCELTQKERARVIDCAYEQTKITYGKLRKLLDLKDTESFAGLTYGLNRSRNKTEDTVFVEMKFYHEVRKALQRAGVFIQDLSIETLDQIGWILSVWKSDDNRRKKLSTLGLSDNVIEELLPLNGSKFGHLSLKAIRKILPFLEDGYSYDVACELAGYQFQGKTEYVKQRLLPPLGEGEVTNPVVRRALSQAIKVVNAVIRKHGSPESIHIELARELSKNLDERRKIEKAQKENQKNNEQIKDEIREILGSAHVTGRDIVKYKLFKQQQEFCMYSGEKLDVTRLFEPGYAEVDHIIPYGISFDDSYDNKVLVKTEQNRQKGNRTPLEYLRDKPEQKAKFIALVESIPLSQKKKNHLLMDKRAIDLEQEGFRERNLSDTRYITRALMNHIQAWLLFDETASMRSKRVVCVNGAVTAYMRARWGLTKDRDAGDKHHAADAVVVACIGDSLIQRVTKYDKFKRNALADRNRYVQQVSKSEGITQYVDKETGEVFTWESFDERKFLPNEPLEPWPFFRDELLARLSDDPSKNIRAIGLLTYSETEQIDPIFVSRMPTRKVTGAAHKETIRSPRIVKVDDNKGTEIQVVVSKVALTELKLTKDGEIKDYFRPEDDPRLYNTLRERLVQFGGDAKAAFKEPVYKISKDGSVRTPVRKVKIQEKLTLGVPVHGGRGIAENGGMVRIDVFEKGGKYYFVPIYVADVLKRELPNRLATAHKPYSEWRVVDDSYQFKFSLYPNDAVMIKPRKEVDITYKDRKEPVGCRIMYFVSANIASASISLRTHDNSGELEGLGIQGLEVFEKYVVDPLGDTHPVYKERRMPFRVERKMN

SEQ ID NO: 86 - DNA, 48 bp, Artificial Sequence; identical to SEQ ID NO: 63
  Synthetic

SEQ ID NO: 87 - DNA, 27 bp, Artificial Sequence
  Synthetic
ccagcagccaagagggtgaagctggat

SEQ ID NO: 88 - DNA, 21 bp, Artificial Sequence
  Synthetic
ccaaagaagaagcggaaggtc

SEQ ID NO: 89 - DNA, 3573 bp, Artificial Sequence
  Synthetic
atgccagcagccaagagggtgaagctggatccagcagccaagagggtgaagctggatggtatccacggagtcccagcagccgcctacagactgggcctggacatcggcatcacctccgtgggatgggcagtggtggccctggagaaggacgagtctggcctgaagcccgtgcggatccaggatctgggcgtgcgcatctttgacaaggccgaggatagcaagaccggcgcctccctggccctgcctaggagagaggcacggagcgcccggcgcaggacaagacggcgcaggcacaggctgtggagggtgaagagactgctggagcagcacggcatcctgtccatggagcagatcgaggccctgtacgcacagcgcaccagctccccagacgtgtatgcactgagggtggcaggactggataggtgcctgatcgccgaggagatcgccagggtgctgatccacatcgcccacagacggggcttccagagcaacagaaagtccgagatcaaggacagcgatgccggcaagctgctgaaggccgtgcaggagaacgagaatctgatgcagtccaagggctaccggacagtggccgagatgctggtgtctgaggccaccaagacagacgccgagggcaagctggtgcacggcaagaagcacggctacgtgagcaacgtgcgcaataaggcaggagagtataggcacaccgtgtccaggcaggcaatcgtggatgaggtgaggaagatctttgcagcacagagagccctgggaaacgacgtgatgtccgaggagctggaggattcttacctgaagatcctgtgcagccagaggaatttcgacgatggaccaggaggcgactccccatacggacacggatccgtgtctcctgatggcgtgaggcagtccatctatgagagaatggtgggctcttgtaccttcgagacaggagagaagagggcaccacgctctagctattccttcgagcggtttcagctgctgaccaaggtggtgaacctgaggatctacagacagcaggaggacggcggccgctatccttgcgagctgacccagacagagcgcgccagagtgatcgattgtgcctacgagcagaccaagatcacatatggcaagctgagaaagctgctggacatgaaggataccgagagctttgccggcctgacatacggcctgaacagatcccggaataagaccgaggacacagtgttcgtggagatgaagttttatcacgaggtgaggaaggccctgcagagagccggcgtgttcatccaggacctgagcatcgagaccctggatcagatcggctggatcctgagcgtgtggaagagcgacgataaccgcaggaagaagctgtctacactgggcctgagcgacaacgtgatcgaggagctgctgcccctgaatggcagcaagtttggccacctgtccctgaaggccatcaggaagatcctgcctttcctggaggacggctactcctatgatgtggcctgcgagctggccggctaccagttccagggcaagaccgagtatgtgaagcagagactgctgccacctctgggagagggagaggtgacaaacccagtggtgagacgggccctgtcccaggccatcaaggtggtgaatgccgtgatccggaagcacggctctcccgagagcatccacatcgagctggcccgcgagctgtctaagaacctggacgagcgcaggaagatcgagaaggcccagaaggagaatcagaagaacaatgagcagatcaaggacgagatcagggagatcctgggatccgcccacgtgaccggaagagatatcgtgaagtacaagctgtttaagcagcagcaggagttctgtatgtattctggcgagaagctggacgtgacacggctgtttgagccaggctacgccgaggtggatcacatcatcccctacggcatctccttcgacgattcttatgacaacaaggtgctggtgaaaaccgagcagaaccggcagaagggcaatcgcacaccactggagtatctgcgggataagcccgagcagaaggccaagtttatcgccctggtggagagcatccccctgtcccagaagaagaagaaccacctgctgatggacaagcgcgccatcgatctggagcaggagggctttcgcgagaggaacctgagcgacaccaggtacatcacaagagccctgatgaatcacatccaggcctggctgctgttcgatgagaccgcctccacacggtctaagagggtggtgtgcgtgaacggagcagtgaccgcctatatgagagcccggtggggcctgacaaaggacaggaatgcaggcgataagcaccacgcagcagacgcagtggtggtggcctgtatcggcgatagcctgatccagagggtgaccaagtacgacaagttcaagagaaacgccctggccgatcgcaatagatacgtgcagcaggtgagcaagtccgagggcatcacacagtatgtggacaaggagaccggcgaggtgttcacatgggagtcctttgatgagaggaagttcctgcctaacgagccactggagccatggcccttctttagggacgagctgctggcaaggctgtctgacgatcctagcaagaatatcagagccatcggcctgctgacctactctgagacagagcagatcgatcccatctttgtgagccggatgcctaccaggaaggtgacaggagcagcacacaaggagaccatcaggtctccaagaatcgtgaaggtggacgataacaagggcacagagatccaggtggtggtgagcaaggtggccctgaccgagctgaagctgacaaaggacggcgagatcaaggattacttcaggcccgaggacgatcctagactgtataataccctgagagagcggctggtgcagtttggaggcgacgcaaaggcagccttcaaggagcccgtgtacaagatctccaaggatggctctgtgaggaccccagtgagaaaggtgaagatccaggagaagctgacactgggagtgccagtgcacggaggaaggggaatcgccgagaacggcggcatggtgcgcatcgacgtgtttgccaaggatggcaagtactatttcgtgcccatctacgtggccgacgtgctgaagagggagctgccaaacagactggcaaccgcacacaagccttattctgagtggagggtggtggacgattcttaccagttcaagtttagcctgtatcctaatgatgccgtgatgatcaagccaagcagagaggtggacatcacctacaaggataggaaggagccagtgggactgaggctgatgtatttcgcaggagcagacatctctggagccaccctgacactgcggacacacgataacagcggagagctgaagggactgggaatccagggcctggaggtgtttgagaagtacgtggtgggacctctgggcgacacccacccagtgtataaggagagacggatgcctttccgggtggagcgcaagatgaatgctagcaaaaggccggcggccacgaaaaaggccggccaggcaaaaaagaaaaagtaa